(19) United States
(12) Patent Application Publication
(10) Pub. No.: US 2015/0111815 A1
(43) Pub. Date: Apr. 23, 2015

(54) NOVEL CYCLOSPORIN DERIVATIVES FOR THE TREATMENT AND PREVENTION OF VIRAL INFECTIONS

(71) Applicant: S&T Global Inc., Woburn, MA (US)

(72) Inventors: Zhuang SU, Andover, MA (US); Zhengyu LONG, Bolton, MA (US); Zhennian HUANG, Newton, MA (US); Suizhou YANG, Dracut, MA (US)

(21) Appl. No.: 14/523,604

(22) Filed: Oct. 24, 2014

Related U.S. Application Data
(63) Continuation of application No. 14/239,698, now abandoned.
(60) Provisional application No. 61/525,289, filed on Aug. 19, 2011.

(51) Int. Cl. C07K 7/64

(52) U.S. Cl. CPC C07K 7/645
USPC 514/3.8; 530/321; 514/20.5; 514/3.7; 514/4.3

(57) ABSTRACT

The present invention relates to a compound of formula (I):

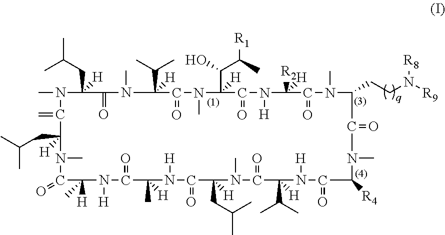

or pharmaceutically acceptable salt thereof, wherein the symbols are as defined in the specification; a pharmaceutical composition comprising the same; a method for treating or preventing a viral infection using the same.

### CROSS REFERENCE TO RELATED APPLICATIONS

This application is a continuation of U.S. application Ser. No. 14/239,698, filed Feb. 19, 2014, which is a continuation of PCT International Application No. PCT/US12/51572, filed Aug. 20, 2012, which claims priority to U.S. Provisional Application No. 61/525,289, filed Aug. 19, 2011, the entire contents of which are hereby incorporated by reference herein.

### FIELD OF INVENTION

The invention relates to novel cyclosporine derivatives, their pharmaceutical compositions comprising the same, and methods for treating or preventing a viral infection using the same.

### BACKGROUND OF THE INVENTION

Cyclosporins in nature are poly-N-methyl, cyclic undecapeptides, isolated from fungi. Cyclosporin A has an immunosuppressive activity and has been used for almost 30 years to prevent rejection in kidney, heart and liver transplant recipients. It possesses anti-inflammatory properties and has been used for treating severe rheumatoid arthritis, severe psoriasis, Behget's uveitis, and dry eye disease. In addition, it is useful for treating severe ulcerative colitis, Crohn's disease, alopecia greata, aplastic anemia, HSV-1 stromal keratitis, systemic lupus erythematosus, and severe lupus nephritis. However, its strong immunosuppressive activity limits its applications in many diseases.

The anti-HIV activity of cyclosporin A was first discovered in 1986 and has been continually studied since then (Klatzmann, D., et al., 1986, C R Acad. Sci. III, 303(9):343-8; Wainberg, M. A., et al., 1988, Blood, 72, 1904-10; Luban, J., et al., 1993, Cell, 73, 1067-1078; each of which is incorporated herein by reference). Its non-immunosuppressive derivative, NIM-811, was reported to have potent anti HIV activity due to its ability to inhibit cyclophilin A (Franke, E. K., et al., 1994, Nature, 372, 359-362; Thali, M., et al., 1994, Nature, 372, 363-365; Gamble, T. R., et al., 1996, Cell, 87, 1157-1159; Rosenwirth B., et al., 1994, Antimicrob. Agents Chemother., 38, 1763-1772; each of which is incorporated herein by reference).

Cyclosporin A and its non-immunosuppressive derivatives, as such as NIM-811 (N-MeIle-4-Cyclosporin), Debio-025, and SCY-635, bind and inhibit cyclophilins; cyclophilins interact with HCV protein NS5A and NS5B and stimulate its RNA-binding activity. As a result, these compounds have an effective anti-HCV activity (Watashi, K., et al., 2007, Rev. Med. Virol., 17:245-252.37; Inoue, K., et al., 2001, Nippon Rinsho., 59, 1326-30; Inoue, K., et al., 2003, J. Gastroenterol., 38, 567-72; Watashi, K., et al., 2003, Hepatology, 38, 1282-8; Gaither, L. A., et al., 2010, Virology, 397, 43-55; each of which is incorporated herein by reference). Currently, NIM-811, Debio-025, and SCY-635 are undergoing clinical trials for treating HCV.

NIM-811 and Debio-025 have a chemical structure similar to cyclosporine A and possess a poor pharmacokinetic profile. In addition, they are metabolized by P450 for inducing drug interactions (Lill, J., et al., 2000, Am J Health-Syst Pharm 57, 1579; incorporated herein by reference).

SCY-635 has an improved pharmacokinetic profile and low blood serum binding. In addition, it has a low potential for drug-drug interactions. SCY-635's in vitro anti-HCV activity (EC₅₀) was reported to be 0.10 μM (Hopkins, S. et al., 2010, Antimicrob. Agents Chemother., 54, 660-672, incorporated herein by reference). However, SCY-635 is not chemically stable, as it is easily converted to its diastereoisomer by epimerization. Its diasteroisomer is expected to have poor binding activity with cyclophilins, and as a result, its anti-viral activity in vivo may be affected (See, e.g., WO2012/009715, WO2012/021796, and WO2012/075494, each of which incorporated herein by reference in its entirety).

Cyclosporin A and its non-immunosuppressive derivatives were also found to possess anti-HBV activity through the inhibition of cyclophilins (Chokshi, S., et al., 2012, Gut 61:A11; Chokshi, S., et al., 2012, Poster Presentations, 47th Annual Meeting of the European Association for the Study of the Liver (EASL 2012), Barcelona, Spain; Chokshi, S., et al., 2011, Abstract 190 (Poster Presentations), 46th Annual Meeting of the European Association for the Study of the Liver (EASL 2011), Berlin, March 30-April 3; Tian, X. C., et al., 2010, J. Virol., 84, 3373-3381; Xia, W. L., et al., 2004, Hepatobiliary Pancreat Dis Int., 4, 18-22; Michael, J., et al., 2003, J. Virol., 77, 7713-7719; each of which is incorporated herein by reference).

Furthermore, cyclophilins were reported to regulate the life cycle and pathogenesis of several viruses, including severe acute respiratory syndrome coronavirus, vaccinia virus, and herpes simplex virus (Castro, A. P., et al., 2003, J. Virol., 77, 9052-9068; Chen, Z., L., et al., 2005, J. Infect. Dis. 191(5):755-760; Arai, C., et al., Nihon Rinsho Meneki Gakkai Kaishi., 35(1), 87-91; Labetoulle, M., 2012, J Fr Ophtalmol., 35(4), 292-307; De Clercq, E., 2008, Expert Opin Emerg Drugs., 13(3):393-416; Vahlne, A., 1992, Arch Virol., 122(1-2):61-75; each of which is incorporated herein by reference). Cyclosporin A and its non-immunosuppressive derivatives also possess such anti viral-activities.

N-MeVal-4-Cyclosporin (SDZ 220-384), another non-immunosuppressive cyclosporine derivative, was reported to have similar biological activities to that of NIM-811 (Fliri, H., et al., 1993, Ann. NY Acad Sci. 696, 47-53; Zenke, G., et al., 1993, Ann NY Acad Sci. 23; 685:330-5).

Hepatitis C virus (HCV) is a small (55-65 nm in size), enveloped, positive sense single strand RNA virus in the Flaviviridae family. HCV has a high rate of replication and an exceptionally high mutation rate. About 80% of people infected with HCV develop chronic, persistent infection. More than 4 million Americans have been infected with HCV and more than 200 million people are estimated to be infected chronically worldwide. About 35,000 new cases of hepatitis C are estimated to occur in the United States each year. HCV infection is responsible for about 50% of all chronic liver disease, 30% of all liver transplants, and 30% of all cirrhosis, end-stage liver disease, and liver cancer in the U.S. The peg-interferon and ribavirin combination is the standard treatment for chronic hepatitis C, but it has low efficacy against HCV infection. Recently, the FDA has approved Vertex's Incivek (telaprevir) and Merck's Victrelis (boceprevir) as an add-on to the current interferon/ribavirin therapy for treating HCV. Both drugs are HCV protease inhibitors that target the virus to prevent its replication. However, due to HCV's fast mutation rate, drug resistance can be developed in a short period of time. Thus, there exists a need for an effective therapeutic for HCV treatment.

Hepatitis B virus (HBV) is a 42 nm partially double stranded DNA virus composed of a 27 nm nucleocapsid core (HBcAg) that is surrounded by an outer lipoprotein envelope containing the surface antigen (HBsAg). More than 2 billion people have been infected, and there are 350 million chronic carriers of the virus. The disease has caused epidemics in parts of Asia and Africa. Chronic hepatitis B will cause liver cirrhosis and liver cancer, a fatal disease with a very poor response to current chemotherapies. The infection is preventable by vaccination, and HBV load and replication can be reduced by current antiviral drugs, such as lamivudine (Epivir), adefovir (Hepsera), tenofovir (Viread), telbivudine (Tyzeka), entecavir (Baraclude), and the two immune system modulators interferon alpha-2a and PEGylated interferon alpha-2a (Pegasys). However, none of the available drugs can clear the infection. There remains a need for an effective therapeutic to treat HBV infection.

The non-immunosuppressive cyclosporin derivatives bind to cyclophilins, a family of host proteins that catalyze cis-trans peptidyl-prolyl isomerization in protein folding and regulation, which are crucial for the processing and maturation of the viral proteins for viral replication. HIV and HCV are viruses with a high mutation rate. All current anti-viral drugs target the virus itself; when the virus mutates, it leads to the development of drug resistance. Instead of directly targeting the virus, targeting host cofactors (cyclophilins) will be slow down the development of drug resistance due to a higher genetic barrier (Rosenwirth, B., et al., 1994, Antimicrob. Agents Chemother., 38, 1763-1772; Tang, H. L. et al., 2010, Viruses, 2, 1621-1634; Hopkins, S. et al., 2010, Oral Presentation, Scynexis's SCY-635 Demonstrates Impressive Barrier to Resistance in HCV Treatment, the 45th Annual Meeting of the European Association for the Study of the Liver (EASL 2010), Vienna, Austria, April 14-18; each of which is incorporated herein by reference). Cyclosporine derivatives affect a new target, cyclophilins, and therefore represent a new mechanism of action against viruses.

There are 17 cyclophilins in the human genome, but the functions of these cyclophilin isoforms are still unclear (Davis, T. L., et al., 2010, PLoS Biol. 8(7):e1000439; incorporated herein by reference). Cyclophilin A, B, C, D, and other such isoforms play an important role in the pathophysiology of a number of serious diseases, such as cancer (Campa, M. J., et al., 2003, Cancer Res., 1; 63(7), 1652-6; Li, M., et al., 2006, Cancer, 106: 2284-94; Yang, H., et al., 2007, Biochem Biophys Res Commun., 361(3):763-7; Mikuriya, K., et al., 2007, Int J. Oncol., 30(4), 849-55; Obchoei, S., et al., 2009, Med Sci Monit., 15(11), RA221-32; Andersson, Y., et al., 2009, Br J Cancer, 101, 1307-1315; Lee, J., 2010, Arch Pharm Res., 33(2), 181-7; Lee, J., et al., 2010, J Exp Clin Cancer Res., 29:97; Obchoei, S., 2011, Molecular Cancer, 10:102; Takahashi, M., et al., 2012, Oncol Rep., 27(1):198-203; each of which is incorporated herein by reference), inflammation (the result of interactions between a secreted extracellular cyclophilin and CD-147, a surface protein; Yurchenko V., 2005, Immunology, 117(3):301-9; Yurchenko, V., 2010, Clin Exp Immunol., 160(3):305-17; Malesević, M., 2010, Angew Chem Int Ed Engl., 49(1):213-5; each of which is incorporated herein by reference), rheumatoid arthritis (Wells, G., et al., 2000, Cochrane Database Syst Rev., (2):CD001083; Kim, H., et al., 2005, Clin Immunol., 116(3):217-24; Yang, Y., Rheumatology (Oxford), 47(9):1299-310; Yurchenko, V., et al., 2006, Immunology, 117(3):301-9; Damsker, J. M., 2009, Immunology, 126(1):55-62; Wang, L., et al., 2010, J Clin Immunol., 30(1):24-33; Billich A., et al., 1997, J Exp Med., 185:975-80; De Ceuninck F., et al., 2003, Arthritis Rheum., 48:2197-206; each of which is incorporated herein by reference), respiratory inflammation (Foda, H. D., et al., 2001, Am J Respir Cell Mol Biol., 25:717-24; Hasaneen, N. A., et al., FASEB J., 19:1507-9. Yurchenko, V., et al., 2006, Immunology, 117(3):301-9; Gwinn, W. M., 2006, J Immunol., 177(7):4870-9; Onoue, S., 2009, J Control Release., 138(1):16-23; Balsley, M. A., et al., 2010, J Immunol., 185(12):7663-70; Balsley, M., et al., 2010, Am. J. Respir. Crit. Care Med., 181(1): A6821; Stemmy, E. J., et al., 2011, J. Asthma, 48(10):986-993; Stemmy, E. J., et al., 2011, Am J Respir Cell Mol Biol., 45(5):991-8; Amin, K., 2012, Respir Med., 106(1):9-14; Onoue, S., 2012, Eur J Pharm Biopharm., 80(1):54-60; each of which is incorporated herein by reference), lupus (Harigai M., et al., 1992, Clin Immunol Immunopathol., 63:58-65; Pistol, G., et al., 2007, J Cell Mol Med., 11:339-48; each of which is incorporated herein by reference), psoriasis (Ellis, C. N., 1991, N Engl J Med., 324, 277-284; incorporated herein by reference), atopic dermatitis (Naeyaert, J. M., et al., 1999, Dermatology, 198:145-152; Pacor, M. L., et al., 2001, Recenti Prog Med., 92(6):390-1; Ricci, G., et al., 2009, Drugs, 69(3):297-306; Simon, D., 2011, Curr Probl Dermatol., 41:156-64; each of which is incorporated herein by reference), dry eye disease (Pflugfelder, S. C., 2004, Am J Ophthalmol., 137(2), 337-42; Kymionis, G. D., et a12008, Clin Ophthalmol., 2, 829-836; Kunert, K. S., et al., 2002, Arch Ophthalmol., 120, 330-7; Yavuz, B., et al., 2012, Scientific World Journal. 2012:194848.; each of which is incorporated herein by reference), severe Graves' ophthalmopathy (Prummel, M. F., 1989, N Engl J Med., 321(20), 1353-9; incorporated herein by reference), endogenous uveitis (Nussenblatt, R. B., et al., 1991, Am J. Ophthalmol., 112(2), 138-46; which is incorporated herein by reference), Wegener's granulomatosis (Georganas, C., et al., 1996, Clin Rheumatol., 15(2), 189-92; incorporated herein by reference), vernal keratoconjutivitis (Pucci, N., et al., 2002, Ann Allergy Asthma Immunol., 89, 298-303; incorporated herein by reference), atopic keratoconjutivitis (Akpek, E. K., et al., 2004, Ophthalmology, 111, 476-82; incorporated herein by reference), ligneous conjutivitis (Rubin, B. I., et al., 1991, Am J Ophthalmol., 112, 95-96; incorporated herein by reference), conjuctival linchen planus (Levell, N. J., et al., 1992, Br J Dermatol., 127, 66-7; incorporated herein by reference), and superior limbic keratoconjutivitis (Perry, H. D., et al., 2003, Ophthalmology, 110, 1578-81; incorporated herein by reference), inflammatory bowel disease-Crohn's Disease and Ulcerative Colitis (Sandborn, W. J., 1995, Inflamm Bowel Dis. 1:48-63; Shibolet, O., et al., 2005, Cochrane Database Syst Rev., (1):CD004277; Rufo, P. A., et al., 2006, Paediatr Drugs, 8(5):279-302; Lémann, M., 2007, Bull Acad Natl Med., 191(6):1125-41; Reindl, W., et al., 2007, Gut., 56(7):1019; Akobeng, A K., 2008, Arch Dis Child. 93(9):787-92; Hart, A. L., et al., 2010, Aliment Pharmacol Ther., 32(5):615-27; Cheifetz, A. S., et al., 2011, J Clin Gastroenterol., 45(2):107-12; Sharkey, L., 2011, J Crohns Colitis., 5(2):91-4; Fabro, M., et al., 2011, Curr Drug Targets., 12(10):1448-53; Van Assche, G., et al., 2011, Gut., 60(1):130-3;. Sinagra, E., et al., 2011, Inflamm Bowel Dis., doi: 10.1002/ibd.21915; Meier, J., et al., World J Gastroenterol., 17(27):3204-12; each of which is incorporated herein by reference), NSAID-induced enteropathy (LoGuidice, A., at al., 2010, Toxicol. Sci., 118, 276-285; which is incorporated herein by reference), cardiovascular diseases (including vascular stenosis, atherosclerosis, abdominal aortic aneurysms, aortic rupture, cardiac hypertrophy, pulmonary arterial hypertension, myocarditis and myocardial fibrosis, and ischaemic heart diseases; Jin, Z. G., et al., 2000, Circ Res., 87(9):789-96; Yurchenko, V., et al., 2005, Immunology, 117, 301 309; Suzuki, J., et al., 2006, Circ Res., 98(6):811-7; Satoh, K., et al., 2008, Circulation., 117(24):3088-98; Nishihara, M., et al., 2008, J Mol Cell Cardiol., 44(2):441-442; Satoh, K., et al., 2010, Circ J., 74(11):2249-56; Satoh, K., et al., 2010, Antioxid Redox Signal., 12(5):675-82; Hausenloy, D. J., et al., 2012, Br J Pharmacol. 165(5):1235-45; Spinale, F. G., et al., 2000, Circulation, 102:1944-9; Siwik, D. A., et al., 2002, Am J Pathol., 160:641-54; Coppinger, J. A., et al., 2004, Blood, 103(6):2096-104; Yoon, Y. W., et al., 2005, Atherosclerosis, 180:37-44; Choi, E. Y., 2002, Exp Mol Med., 34:391-400; Satoh, K., et al., 2010, Antioxid Redox Signal., 1:12(5), 675-682; Nigro, P., et al., 2010, J Exp Med., 208(1):53-66; Seizer P., 2011, Int J Cardiol., 2011 Jul. 1.; Wang, W. L., et al., 2011, Med Hypotheses, 77(5):734-8; Hattori, F., 2012, J Mol Cell Cardiol., 2012 Apr. 21; Seizer P., 2012, J Mol Cell Cardiol., 2012 Mar. 15.; each of which is incorporated herein by reference), severe sepsis (Tegeder, I., et al., 1997, J Clin Immunol., 17(5):380-6; Dear, J. W., et al., 2007, Crit Care Med., 35(10):2319-28; each of which is incorporated herein by reference), ischaemic brain diseases (Boulos, S., et al., 2007, Neurobiol Dis., 25:54-64; incorporated herein by reference), and Lyme disease (Kratz, A., et al., 1992, Clin Exp Immunol., 90:422-7; incorporated herein by reference). The cyclosporin derivatives of the present invention bind and inhibit cyclophilins and therefore can be used for treatment of the diseases mentioned herein.

Due to cyclophilin inhibition, cyclosporin derivatives also possess the following activities: anti-fungal (Kirkland, T. N., et al., 1983, Antimicrob Agents Chemother., 24(6): 921-924; Osato, M S., et al., 1983, Transplant Proc., 15, no. 4 supp.l. 1983; Mody, C. H., et al., 1988, Infect Immun., 56(1): 7-12; Roilides, E., et al., 1994, Antimicrob Agents Chemother., 38(12): 2883-2888; Moussaïf, M., et al., 1997, Appl Environ Microbiol., 63(5):1739-43; Cruz, M. C., et al., 2000, Antimicrob Agents Chemother., 44(1):143-9; each of which is incorporated herein by reference), anti-malarial (Nickell, S. P., et al., 1982, Infect Immun., 37(3):1093-100; Murphy, J. R., et al, 1988, Antimicrob Agents Chemother., 32(4):462-6; Schlesinger, P. H., et al., 1988, Antimicrob Agents Chemother., 32(6):793-798; Marín-Menéndez, A., et al., 2012, Mol Biochem Parasitol., 184(1):44-7; each of which is incorporated herein by reference), and anti-parasitic (including Leishmania donovani, Cryptosporidium parvum, Hymenolepis nana, Toxoplasma, Trypanosoma cruzi, and Schistosome; Chappell, L. H., et al., 1992, Parasitology, 1992; 105 Suppl:S25-40; Bell, A., et al., 1996, Gen Pharmacol., 27(6):963-71; Yau, W. L., et al., 2010, PLoS Negl Trop Dis., 4(6):e729; Yurchenko, V., et al., 2008, Int J Parasitol., 38(6):633-9; Perkins, M. E., et al., 1998, Antimicrob Agents Chemother., 42(4):843-8; Matsuzawa, K., et al., 1998, Int J Parasitol., 28(4):579-88; Silverman, J. A., et al., 1997, Antimicrob Agents Chemother., 41(9):1859-66; Búa, J., et al., 2008, Parasitology, 135(2):217-28; Búa, J., et al., 2004, Bioorg Med Chem Lett., 14(18):4633-7; Bout, D. T, et al., Am J Trop Med Hyg., 33(1):185-6; Bout, D., et al., 1986, Infect Immun., 52(3):823-7; Munro, G. H., et al., Parasitology, 102 Pt 1:57-63; each of which is incorporated herein by reference). Cyclosporin derivatives also promote hair growth (Watanabe, S., et al., 1991, J Dermatol., (12):714-9; Paus R., et al., 1994, J Invest Dermatol., 103:2, 143-7; Hozumi, Y., et al., 1994, J Dermatol Sci., 7 Suppl:, S33-8; Takahashi, T., et al., 2001, J Invest Dermatol., 117(3):605-11; Taylor M., et al., 1993, J Invest Dermatol., 100:3, 237-9; Gafter-Gvili, A., et al., 2004, Arch Dermatol Res., 296(6):265-9; each of which is incorporated herein by reference).

Recent research for Alzheimer's disease indicated that Cyclophilin A is also a key target for treating APOE4-mediated neurovascular injury and the resulting neuronal dysfunction and degeneration (Bell, R. D., et al., 2012, Nature, 485(7399):512-6; Bell, R. D., et al., 2009, Acta Neuropathol., 118(1):103-13; each of which is incorporated herein by reference).

Due to the function of extracellular cyclophilins, it is necessary to emphasize that the special target of a secreted extracellular cyclophilin using a cell-impermeable derivative of cyclosporine will be very effective in reducing inflammation for diseases such as respiratory inflammation and cardiovascular diseases (Yurchenko V., 2005, Immunology, 117(3):301-9; Yurchenko, V., 2010, Clin Exp Immunol., 160(3):305-17; Malesević, M., 2010, Angew Chem Int Ed Engl., 49(1):213-5; Balsley, M. A., et al., 2010, J Immunol., 185(12):7663-70; Balsley, M., et al., 2010, Am. J. Respir. Grit. Care Med., 181(1): A6821; Satoh, K., et al., 2010, Circ J., 74(11):2249-56; each of which is incorporated herein by reference).

Cyclophilin D (CypD) is very important for mitochondrial related neuro and cardiovascular functions because it is an integral part of the mitochondrial permeability transition pore (mPTP). Unregulated opening of the mPTP can lead to mitochondrial swelling and cell death. Thus, the CypD-mediated mPTP is directly linked to a new pharmacologic treatment strategy for many neuro and cardiovascular diseases, such as Alzheimer's disease, Parkinson's disease, Huntington's disease, ALS, aging, heart attack, heart failure, traumatic brain injury, spinal cord injury, epilepticus, stroke, ischemia-reperfusion injury in the brain, heart, kidney, and particularly in myocardial infarction. The CypD-mediated mPTP is also linked to a new treatment strategy for obesity, diabetes, and muscular dystrophy (Henry-Mowatt, J., 2004, Oncogene, 23, 2850-60; Galluzzi, L., 2006, Oncogene, 25, 4812-4830; Guo, X., et al., 2001, Eur J Neurosci., 13, 1683-1693; Hirai, K., et al., 2001, J Neurosci., 21, 3017-3023; Friberg, H., et al., 2002, Biochimie, 84, 241-250; Waldmeier, P. C., et al., 2003, Curr Med Chem., 10, 1485-506; Hansson, M. J., et al., 2004, J Bioenerg Biomembr., 36, 407-13; Lifshitz, J., et al, 2004, Mitochondrion, 4, 705-13; Sullivan, P. G., et al., 2005, J Neurosci Res., 79, 231-9; Baines, C. P., et al, 2005, Nature 434, 658-662; Shanmuganathan, S., et al, 2005, Am J Physiol Heart Circ Physiol., 289, H237-H242; McBride, H. M., et al., 2006, Curr Biol., 16, 551-560; Mandemakers, W., et al., 2007, J Cell Sci., 120, 1707-1716; Kroemer, G., et al., 2007, Physiol Rev., 87, 99-163; Ibarra, A., et al., 2007, Brain Res., 1149, 200-209; Michelakis, E. D., et al, 2008, Circulation, 117, 2431-2434; Du, H., et al, 2008, Nature Medicine, 14, 1097-1105; Piot C., et al., 2008, N Engl J Med., 359, 473-81; Hatton, J., et al., 2008, J Neurosurg., 109, 699-707; Tatsuta, T., et al., 2008, EMBO J, 27, 306-314; Reutenauer, J., et al., 2008, Br J Pharmacol., 155, 574-84; Mazzeo, A. T., et al., 2009, Exp Neurol., 218, 363-370; Galluzzi, L., et al, 2009, Nature Rev Neurosci., 10, 481-494; Halestrap, A. P., et al., 2009, Biochim Biophys Acta., 1787, 1402-15; Arnett, A. L. H., et al., 2009, Curr. Opin. Genet. Dev., 19, 290-297; Tiepolo, T., et al., 2009, Br J Pharmacol., 157, 1045-1052; Wissing, E. R., et al., 2010, Neuromuscul Disord., 20, 753-60; Halestrap, A. P., et al., 2010, Biochem Soc Trans., 38, 841-860; Cernak, I., et al., 2010, J Cereb Blood Flow Metab., 30, 255-66; Elrod, J. W., et al., 2010, J Clin Invest., 120, 3680-3687; Duchen, M. R., et al., 2010, Essays Biochem., 47, 115-37; Schapira, A. H. V., et al., 2011, Parkinson's Disease, Volume 2011, 1-7 Article ID 159160; Osman, M. M., et al., 2011, Neuropeptides, 45, 359-368; Devalaraja-Narashimha K., et al., 2011, FEBS Lett., 585, 677-82; Fujimoto, K., et al., 2010, Proc Natl Acad Sci USA. 107, 10214-9; Irwin, W. A., et al., 2003, Nat Genet., 35, 267-271; Angelin, A., et al., 2007, Proc Natl Acad Sci USA, 104, 991-6; Merlini, L., et al., 2008, Proc Natl Acad Sci USA, 105, 5225-9; Millay, D. P., 2008, Nat Med., 14, 442-7; each of which is incorporated herein by reference). Cyclosporine A and its derivatives can block CypD to prevent mitochondrial swelling and cell death, and therefore could be useful for treatment of the aforementioned diseases, for example, as a neuro and cardiovascular protective agent or as a novel mitochondrial medicine.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

### SUMMARY OF THE INVENTION

In one aspect, the present invention provides a compound of the formulae (I):

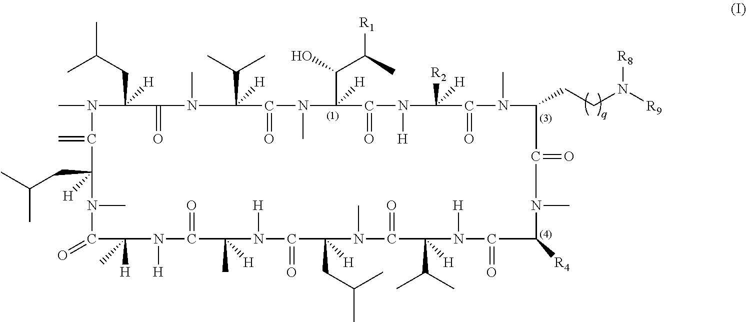

or pharmaceutically acceptable salt thereof, wherein the symbols have the following meanings and are, for each occurrence, independently selected:
R₁ is n-butyl, (E)-but-2-enyl;
R₂ is ethyl, 1-hydroxyethyl, isopropyl or n-propyl;

### R₄ is

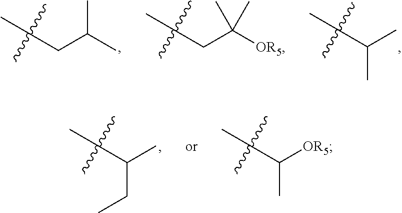

each R₅ is independently H, alkyl or substituted alkyl, alkenyl or substituted alkenyl, alkynyl or substituted alkynyl, cycloalkyl or substituted cycloalkyl, cycloalkenyl or substituted cycloalkenyl, or aryl or substituted aryl; and
each occurrence of R₈ and R₉ is independently H, alkyl or substituted alkyl, alkenyl or substituted alkenyl, alkynyl or substituted alkynyl, cycloalkyl or substituted cycloalkyl, phenyl or substituted phenyl, or R₈ and R₉, together with the nitrogen atom to which they are attached

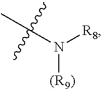

form a heterocycle or substituted heterocycle; and
q is an integer of 0, 1, 2, 3, 4, or 5.

In another aspect, the present invention provides a compound of the formulae (I) as shown above, or pharmaceutically acceptable salt thereof, wherein:
R₁ is n-butyl or (E)-but-2-enyl;
R₂ is ethyl, 1-hydroxyethyl, isopropyl or n-propyl;
R₄ is

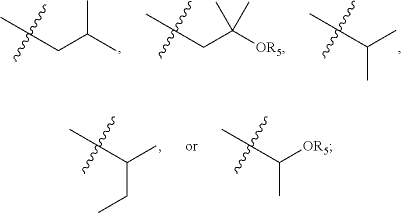

R₅ is:
   H;
   (C₁-C₆)alkyl, optionally substituted by one or more groups R₇ which may be the same or different;
   (C₂-C₆)alkenyl, optionally substituted by one or more groups which may be the same or different selected from hydroxy, (C₁-C₆)alkyl, aryl (e.g., phenyl), (CH₂)ₚOR_{A}, O(CH₂)ₘOH, O(CH₂)ₘO(CH₂)ₘOH, O(CH₂)ₘNR_{A}R_{B}, O(CH₂)ₘO(CH₂)ₘNR_{A}R_{B}, (CH₂)ₚNR_{A}R_{B}, (CH₂)ₚNR_{C}(CH₂)ₘNR_{A}R_{B}, (CH₂)ₚNR_{C}(CH₂)ₘNR_{C}(CH₂)ₘNR_{A}R_{B}, (CH₂)ₚC(═O)NR_{A}R_{B}, (CH₂)ₚC(═O)OR_{A};
   (C₂-C₆)alkynyl, optionally substituted by one or one or more groups which may be the same or different selected from halogen, hydroxy, amino, monoalkylamino and dialkylamino;
   (C₃-C₇)cycloalkyl, optionally substituted by one or more groups which may be the same or different selected from halogen, hydroxy, amino, monoalkylamino and dialkylamino;
   phenyl or CH₂-phenyl, optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B}, (CH₂)ₚC(═O)OR_{A};
each occurrence of R₇ is independently halogen, hydroxy, aryl (e.g., phenyl), S(C₁-C₆)alkyl, SR_{A}, OR_{A}, O(CH₂)ₘOH, O(CH₂)ₘO(CH₂)ₘOH, O(CH₂)ₘO(C₁-C₆)alkyl, O(CH₂)ₘO(CH₂)ₘO(C₁-C₆)alkyl, C(═O)OR_{A}, C(═O)NR_{A}R_{B}, NR_{A}R_{B}, O(CH₂)ₘNR_{A}R_{B}, O(CH₂)ₘO(CH₂)ₘNR_{A}R_{B}, NR_{c}(CH₂)ₘNR_{A}R_{B}, or NR_{c}(CH₂)ₘNR_{c}(CH₂)ₘNR_{A}R_{B}, wherein said aryl or phenyl is optionally substituted by one or more groups which may be the same or different selected from halogen, hydroxy, (C₁-C₆)alkyl, (CH₂)ₚOR_{A}, (CH₂)ₚNR_{A}R_{B}, (CH₂)_{B}C(═O)NR_{A}R_{B} and (CH₂)ₚC(═O)OR_{A};
each occurrence of R₈ and R₉ is independently H, alkyl, alkenyl, alkynyl, cycloalkyl, or phenyl, in which said alkyl, alkenyl, alkynyl, cycloalkyl, and phenyl may be optionally substituted by one or more groups R₁₀ which may be the same or different, in which each occurrence of R₁₀ is independently halogen, hydroxy, O(C₁-C₄)alkyl, C(═O)(C₁-C₄)alkyl, C(═O)O(C₁-C₄)alkyl; or R₈ and R₉, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from (C₁-C₄)alkyl, phenyl and benzyl.
each occurrence of R_{A} and R_{B} is independently:
   hydrogen;
   (C₁-C₆)alkyl, optionally substituted by one or more groups R_{D} which may be the same or different;
   (C₂-C₆)alkenyl or (C₂-C₆)alkynyl;
   (C₃-C₇)cycloalkyl optionally substituted with (C₁-C₆)alkyl;
   phenyl optionally substituted with from one to five groups which may be the same or different selected from halogen, —O(C₁-C₆)alkyl, —C(═O)O(C₁-C₆)alkyl, amino, alkylamino and dialkylamino;
   or a heterocyclic ring which may be saturated or unsaturated containing five or six ring atoms and from one to three heteroatoms which may the same or different selected from nitrogen, sulfur and oxygen;
   or R_{A} and R_{B}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from the group consisting of alkyl, phenyl and benzyl;
each occurrence of R_{c} is independently hydrogen or (C₁-C₆)alkyl;
p is an integer of 0, 1, 2, 3, 4, or 5;
q is an integer of 0, 1, 2, 3, 4, or 5; and
m is an integer of 1, 2, 3, 4 or 5.

In yet another aspect, the present invention provides a pharmaceutical composition comprising at least one compound as described herein and a pharmaceutically-acceptable carrier.

In a further aspect, the present invention provides a method for treating or preventing a viral infection in a mammalian species in need thereof, the method comprising administering to the mammalian species a therapeutically effective amount of at least one compound as described herein.

In another aspect, the present invention provides a method for treating or preventing hepatitis C virus infection in a mammalian species in need thereof, the method comprising administering to the mammalian species a therapeutically effective amount of at least one compound as described herein.

### DETAILED DESCRIPTION OF THE INVENTION

### Definitions

The following are definitions of terms used in the present specification. The initial definition provided for a group or term herein applies to that group or term throughout the present specification individually or as part of another group, unless otherwise indicated.

The terms “alkyl” and “alk” refers to a straight or branched chain alkane (hydrocarbon) radical containing from 1 to 12 carbon atoms, preferably 1 to 6 carbon atoms. Exemplary “alkyl” groups include methyl, ethyl, propyl, isopropyl, n-butyl, t-butyl, isobutyl pentyl, hexyl, isohexyl, heptyl, 4,4-dimethylpentyl, octyl, 2,2,4-trimethylpentyl, nonyl, decyl, undecyl, dodecyl, and the like. The term “(C₁-C₄)alkyl” refers to a straight or branched chain alkane (hydrocarbon) radical containing from 1 to 4 carbon atoms, such as methyl, ethyl, propyl, isopropyl, n-butyl, t-butyl, and isobutyl. The term “(C₁-C₆)alkyl” refers to a straight or branched chain alkane (hydrocarbon) radical containing from 1 to 6 carbon atoms, such as n-hexyl, 2-methylpentyl, 3-methylpentyl, 4-methylpentyl, 2,3-dimethylbutyl, 2,2-dimethylbutyl, in addition to those exemplified for “(C₁-C₄)alkyl.” “Substituted alkyl” refers to an alkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. In the aforementioned exemplary substituents, groups such as alkyl, cycloalkyl, alkenyl, alkynyl, cycloalkenyl, heterocycle and aryl can themselves be optionally substituted.

The term “alkenyl” refers to a straight or branched chain hydrocarbon radical containing from 2 to 12 carbon atoms and at least one carbon-carbon double bond. Exemplary such groups include ethenyl or allyl. The term “C₂-C₆ alkenyl” refers to a straight or branched chain hydrocarbon radical containing from 2 to 6 carbon atoms and at least one carbon-carbon double bond, such as ethylenyl, propenyl, 2-propenyl, (E)-but-2-enyl, (Z)-but-2-enyl, 2-methy(E)-but-2-enyl, 2-methy(Z)-but-2-enyl, 2,3-dimethyl-but-2-enyl, (Z)-pent-2-enyl, (E)-pent-1-enyl, (Z)-hex-1-enyl, (E)-pent-2-enyl, (Z)-hex-2-enyl, (E)-hex-2-enyl, (Z)-hex-1-enyl, (E)-hex-1-enyl-(Z)-hex-3-enyl, (E)-hex-3-enyl, and (E)-hex-1,3-dienyl. “Substituted alkenyl” refers to an alkenyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “alkynyl” refers to a straight or branched chain hydrocarbon radical containing from 2 to 12 carbon atoms and at least one carbon to carbon triple bond. Exemplary such groups include ethynyl. The term “C₂-C₆ alkynyl” refers to a straight or branched chain hydrocarbon radical containing from 2 to 6 carbon atoms and at least one carbon-carbon triple bond, such as ethynyl, prop-1-ynyl, prop-2-ynyl, but-1-ynyl, but-2-ynyl, pent-1-ynyl, pent-2-ynyl, hex-1-ynyl, hex-2-ynyl, hex-3-ynyl. “Substituted alkynyl” refers to an alkynyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted.

The term “cycloalkyl” refers to a fully saturated cyclic hydrocarbon group containing from 1 to 4 rings and 3 to 8 carbons per ring. “C₃-C₇ cycloalkyl” refers to cyclopropyl, cyclobutyl, cyclopentyl, cyclohexyl, or cycloheptyl. “Substituted cycloalkyl” refers to a cycloalkyl group substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “cycloalkenyl” refers to a partially unsaturated cyclic hydrocarbon group containing 1 to 4 rings and 3 to 8 carbons per ring. Exemplary such groups include cyclobutenyl, cyclopentenyl, cyclohexenyl, etc. “Substituted cycloalkenyl” refers to a cycloalkenyl group substituted with one more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cylic substituents, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “aryl” refers to cyclic, aromatic hydrocarbon groups that have 1 to 5 aromatic rings, especially monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). “Substituted aryl” refers to an aryl group substituted by one or more substituents, preferably 1 to 3 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SRₐ, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include fused cylic groups, especially fused cycloalkyl, fused cycloalkenyl, fused heterocycle, or fused aryl, where the aforementioned cycloalkyl, cycloalkenyl, heterocycle and aryl substituents can themselves be optionally substituted.

The terms “heterocycle” and “heterocyclic” refer to fully saturated, or partially or fully unsaturated, including aromatic (i.e., “heteroaryl”) cyclic groups (for example, 4 to 7 membered monocyclic, 7 to 11 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. (The term “heteroarylium” refers to a heteroaryl group bearing a quaternary nitrogen atom and thus a positive charge.) The heterocyclic group may be attached to the remainder of the molecule at any heteroatom or carbon atom of the ring or ring system. Exemplary monocyclic heterocyclic groups include azetidinyl, pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, hexahydrodiazepinyl, 4-piperidonyl, pyridyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include carbazolyl, benzidolyl, phenanthrolinyl, acridinyl, phenanthridinyl, xanthenyl and the like.

“Substituted heterocycle” and “substituted heterocyclic” (such as “substituted heteroaryl”) refer to heterocycle or heterocyclic groups substituted with one or more substituents, preferably 1 to 4 substituents, at any available point of attachment. Exemplary substituents include but are not limited to one or more of the following groups: hydrogen, halogen (e.g., a single halogen substituent or multiple halo substituents forming, in the latter case, groups such as CF₃ or an alkyl group bearing Cl₃), cyano, nitro, oxo (i.e., ═O), CF₃, OCF3, cycloalkyl, alkenyl, cycloalkenyl, alkynyl, heterocycle, aryl, ORₐ, SR, S(═O)Rₑ, S(═O)₂Rₑ, P(═O)₂Rₑ, S(═O)₂ORₑ, P(═O)₂ORₑ, NR_{b}R_{c}, NR_{b}S(═O)₂Rₑ, NR_{b}P(═O)₂Rₑ, S(═O)₂NR_{b}R_{c}, P(═O)₂NR_{b}R_{c}, C(═O)OR_{d}, C(═O)Rₐ, C(═O)NR_{b}R_{c}, OC(═O)Rₐ, OC(═O)NR_{b}R_{c}, NR_{b}C(═O)ORₑ, NR_{d}C(═O)NR_{b}R_{c}, NR_{d}S(═O)₂NR_{b}R_{c}, NR_{d}P(═O)₂NR_{b}R_{c}, NR_{b}C(═O)Rₐ, or NR_{b}P(═O)₂Rₑ, wherein each occurrence of Rₐ is independently hydrogen, alkyl, cycloalkyl, alkenyl, cycloalkenyl, alkynyl, heterocycle, or aryl; each occurrence of R_{b}, R_{c} and R_{d} is independently hydrogen, alkyl, cycloalkyl, heterocycle, aryl, or said R_{b} and R_{c} together with the N to which they are bonded optionally form a heterocycle; and each occurrence of Rₑ is independently alkyl, cycloalkyl, alkenyl, cycloalkenyl, alkynyl, heterocycle, or aryl. The exemplary substituents can themselves be optionally substituted. Exemplary substituents also include spiro-attached or fused cylic substituents at any available point or points of attachment, especially spiro-attached cycloalkyl, spiro-attached cycloalkenyl, spiro-attached heterocycle (excluding heteroaryl), fused cycloalkyl, fused cycloalkenyl, fused heterocycle, or fused aryl, where the aforementioned cycloalkyl, cycloalkenyl, heterocycle and aryl substituents can themselves be optionally substituted.

The term “alkylamino” refers to a group having the structure —NHR′, wherein R′ is hydrogen, alkyl or substituted alkyl, cycloalkyl or substituted cyclolakyl, as defined herein. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each independently alkyl or substituted alkyl, cycloalkyl or substituted cycloalkyl, cycloalkenyl or substituted cyclolalkenyl, aryl or substituted aryl, heterocyclyl or substituted heterocyclyl, as defined herein. R and R′ may be the same or different in an dialkyamino moiety. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

The terms “halogen” or “halo” refer to chlorine, bromine, fluorine or iodine.

Unless otherwise indicated, any heteroatom with unsatisfied valences is assumed to have hydrogen atoms sufficient to satisfy the valences.

The compounds of the present invention may form salts which are also within the scope of this invention. Reference to a compound of the present invention is understood to include reference to salts thereof, unless otherwise indicated. The term “salt(s)”, as employed herein, denotes acidic and/or basic salts formed with inorganic and/or organic acids and bases. In addition, when a compound of the present invention contains both a basic moiety, such as but not limited to a pyridine or imidazole, and an acidic moiety such as but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful, e.g., in isolation or purification steps which may be employed during preparation. Salts of the compounds of the present invention may be formed, for example, by reacting a compound of the present invention with an amount of acid or base, such as an equivalent amount, in a medium such as one in which the salt precipitates or in an aqueous medium followed by lyophilization.

The compounds of the present invention which contain a basic moiety, such as but not limited to an amine or a pyridine or imidazole ring, may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of the present invention which contain an acidic moiety, such but not limited to a carboxylic acid, may form salts with a variety of organic and inorganic bases. Exemplary basic salts include ammonium salts, alkali metal salts such as sodium, lithium and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Prodrugs and solvates of the compounds of the invention are also contemplated herein. The term “prodrug” as employed herein denotes a compound that, upon administration to a subject, undergoes chemical conversion by metabolic or chemical processes to yield a compound of the present invention, or a salt and/or solvate thereof. Solvates of the compounds of the present invention include, for example, hydrates.

Compounds of the present invention, and salts or solvates thereof, may exist in their tautomeric form (for example, as an amide or imino ether). All such tautomeric forms are contemplated herein as part of the present invention.

All stereoisomers of the present compounds (for example, those which may exist due to asymmetric carbons on various substituents), including enantiomeric forms and diastereomeric forms, are contemplated within the scope of this invention. Individual stereoisomers of the compounds of the invention may, for example, be substantially free of other isomers (e.g., as a pure or substantially pure optical isomer having a specified activity), or may be admixed, for example, as racemates or with all other, or other selected, stereoisomers. The chiral centers of the present invention may have the S or R configuration as defined by the International Union of Pure and Applied Chemistry (IUPAC) 1974 Recommendations. The racemic forms can be resolved by physical methods, such as, for example, fractional crystallization, separation or crystallization of diastereomeric derivatives or separation by chiral column chromatography. The individual optical isomers can be obtained from the racemates by any suitable method, including without limitation, conventional methods, such as, for example, salt formation with an optically active acid followed by crystallization.

Compounds of the present invention are, subsequent to their preparation, preferably isolated and purified to obtain a composition containing an amount by weight equal to or greater than 90%, for example, equal to greater than 95%, equal to or greater than 99% compound of the present invention (“substantially pure”), which is then used or formulated as described herein. Such “substantially pure” compounds of the present invention are also contemplated herein as part of the present invention.

All configurational isomers of the compounds of the present invention are contemplated, either in admixture or in pure or substantially pure form. The definition of compounds of the present invention embraces both cis (Z) and trans (E) alkene isomers, as well as cis and trans isomers of cyclic hydrocarbon or heterocyclic rings.

Throughout the specifications, groups and substituents thereof may be chosen to provide stable moieties and compounds.

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

### Compounds

The novel cyclosporin derivatives of the present invention are potent inhibitors of viruses such as HIV, HBV and HCV.

In one aspect, the present invention provides a compound of the formula (I):

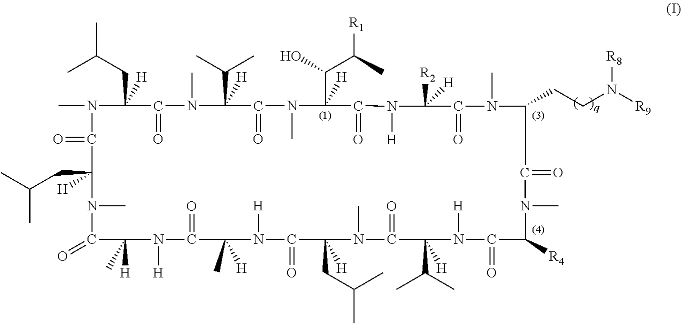

or pharmaceutically acceptable salt thereof, wherein the symbols have the following meanings and are, for each occurrence, independently selected:
R₁ is n-butyl, (E)-but-2-enyl;
R₂ is ethyl, 1-hydroxyethyl, isopropyl or n-propyl;

### R₄ is

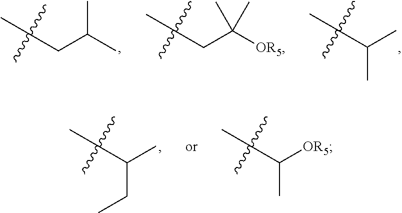

each R₅ is independently H, alkyl or substituted alkyl, alkenyl or substituted alkenyl, alkynyl or substituted alkynyl, cycloalkyl or substituted cycloalkyl, cycloalkenyl or substituted cycloalkenyl, or aryl or substituted aryl; and
each occurrence of R₈ and R₉ is independently H, alkyl or substituted alkyl, alkenyl or substituted alkenyl, alkynyl or substituted alkynyl, cycloalkyl or substituted cycloalkyl, phenyl or substituted phenyl, or R₈ and R₉, together with the nitrogen atom to which they are attached

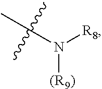

form a heterocycle or substituted heterocycle; and
q is an integer of 0, 1, 2, 3, 4, or 5.

In another aspect, the present invention provides a compound of formula (I) as shown above, or pharmaceutically acceptable salt thereof, wherein:
R₁ is n-butyl or (E)-but-2-enyl;
R₂ is ethyl, 1-hydroxyethyl, isopropyl or n-propyl;
R₄ is

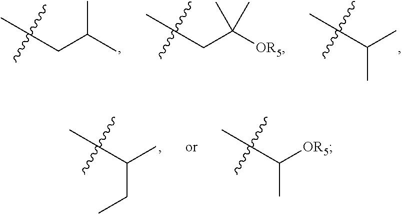

R₅ is:
   H;
   (C₁-C₆)alkyl, optionally substituted by one or more groups R₇ which may be the same or different;
   (C₂-C₆)alkenyl, optionally substituted by one or more groups which may be the same or different selected from hydroxy, (C₁-C₆)alkyl, aryl (e.g., phenyl), (CH₂)ₚOR_{A}, O(CH₂)ₘOH, O(CH₂)ₘO(CH₂)ₘOH, O(CH₂)ₘNR_{A}R_{B}, O(CH₂)ₘO(CH₂)ₘNR_{A}R_{B}, (CH₂)ₚNR_{A}R_{B}, (CH₂)ₚNR_{C}(CH₂)ₘNR_{A}R_{B}, (CH₂)ₚNR_{C}(CH₂)ₘNR_{C}(CH₂)ₘNR_{A}R_{B}, (CH₂)ₚC(═O)NR_{A}R_{B}, (CH₂)ₚC(═O)OR_{A};
   (C₂-C₆)alkynyl, optionally substituted by one or one or more groups which may be the same or different selected from halogen, hydroxy, amino, monoalkylamino and dialkylamino;
   (C₃-C₇)cycloalkyl, optionally substituted by one or more groups which may be the same or different selected from halogen, hydroxy, amino, monoalkylamino and dialkylamino;
   phenyl or CH₂-phenyl, optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B}, (CH₂)ₚC(═O)OR_{A};
each occurrence of R₇ is independently halogen, hydroxy, aryl (e.g., phenyl), S(C₁-C₆)alkyl, SR_{A}, OR_{A}, O(CH₂)ₘOH, O(CH₂)ₘO(CH₂)ₘOH, O(CH₂)ₘO(C₁-C₆)alkyl, O(CH₂)ₘO(CH₂)ₘO(C₁-C₆)alkyl, C(═O)OR_{A}, C(═O)NR_{A}R_{B}, NR_{A}R_{B}, O(CH₂)ₘNR_{A}R_{B}, O(CH₂)ₘO(CH₂)ₘNR_{A}R_{B}, NR_{C}(CH₂)ₘNR_{A}R_{B}, or NR_{c}(CH₂)ₘNR_{c}(CH₂)ₘNR_{A}R_{B}, wherein said aryl or phenyl is optionally substituted by one or more groups which may be the same or different selected from halogen, hydroxy, (C₁-C₆)alkyl, (CH₂)_{B}OR_{A}, (CH₂)ₚNR_{A}R_{B}, (CH₂)_{B}C(═O)NR_{A}R_{B} and (CH₂)ₚC(═O)OR_{A};
each occurrence of R₈ and R₉ is independently H, alkyl, alkenyl, alkynyl, cycloalkyl, or phenyl, in which said alkyl, alkenyl, alkynyl, cycloalkyl, and phenyl may be optionally substituted by one or more groups R₁₀ which may be the same or different, in which each occurrence of R₁₀ is independently halogen, hydroxy, O(C₁-C₄)alkyl, C(═O)(C₁-C₄)alkyl, C(═O)O(C₁-C₄)alkyl; or R₈ and R₉, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from (C₁-C₄)alkyl, phenyl and benzyl.
each occurrence of R_{A} and R_{B} is independently:
   hydrogen;
   (C₁-C₆)alkyl, optionally substituted by one or more groups R_{D} which may be the same or different;
   (C₂-C₆)alkenyl or (C₂-C₆)alkynyl;
   (C₃-C₇)cycloalkyl optionally substituted with (C₁-C₆)alkyl;
   phenyl optionally substituted with from one to five groups which may be the same or different selected from halogen, —O(C₁-C₆)alkyl, —C(═O)O(C₁-C₆)alkyl, amino, alkylamino and dialkylamino;
   or a heterocyclic ring which may be saturated or unsaturated containing five or six ring atoms and from one to three heteroatoms which may the same or different selected from nitrogen, sulfur and oxygen;
   or R_{A} and R_{B}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from the group consisting of alkyl, phenyl and benzyl;
each occurrence of R_{c} is independently hydrogen or (C₁-C₆)alkyl;
q is an integer of 0, 1, 2, 3, 4, or 5;
p is an integer of 0, 1, 2, 3, 4, or 5; and
m is an integer of 1, 2, 3, 4 or 5.

In certain embodiments, R₁ is n-butyl or

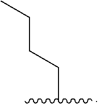 [SMILES: *CCCC]

In certain other embodiments, R₁ is (E)-but-2-enyl or

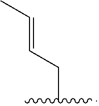 [SMILES: *C/C=C/C]

In certain embodiments, R₂ is ethyl. In certain other embodiments, R₂ is 1-hydroxyethyl. In yet other embodiments, R₂ is isopropyl. In yet other embodiments, R₂ is n-propyl.

In certain embodiments, q is 1, 2, or 3.

In certain embodiments, m is 1. In certain other embodiments, m is 2. In yet other embodiments, m is 3. In yet other embodiments, m is 4 or 5.

In certain embodiments, p is 0. In certain other embodiments, p is 1. In yet other embodiments, m is 2. In yet other embodiments, m is 3, 4 or 5.

In certain embodiments, each occurrence of R₈ and R₉ is independently hydrogen; (C₁-C₄)alkyl, optionally substituted by one or more groups R₁₀ which may be the same or different, in which each occurrence of R₁₀ is independently halogen, hydroxy, O(C₁-C₄)alkyl, C(═O)(C₁-C₄)alkyl, C(═O)O(C₁-C₄)alkyl; or R₁₀ and R₁₀, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from (C₁-C₄)alkyl, phenyl and benzyl.

In certain embodiments, R₈ and R₉, together with the nitrogen atom to which they are attached, form a saturated or unsaturated heterocyclic ring containing from three to seven ring atoms, which ring may optionally contain another heteroatom selected from nitrogen, oxygen and sulfur and may be optionally substituted by from one to four groups which may be the same or different selected from (C₁-C₄)alkyl, phenyl and benzyl.

In certain embodiments,

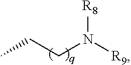

at (3)-position is 2-aminoethyl, 2-aminopropyl, 3-aminopropyl, 2-monoalkylaminoethyl, 2-monoalkylaminopropyl, 3-monoalkylaminopropyl, 2-dialkylaminoethyl, 2-dialkylaminopropyl, or 3-dialkylaminopropyl, wherein said alkyl is (C₁-C₄)alkyl.

In certain embodiments,

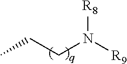

at (3)-position is 2-aminoethyl, 2-aminopropyl, 3-aminopropyl, 2-monoalkylaminoethyl, 2-monoalkylaminopropyl, 3-monoalkylaminopropyl, 2-dialkylaminoethyl, 2-dialkylaminopropyl, or 3-dialkylaminopropyl, wherein said alkyl is (C₁-C₄)alkyl. wherein R₃ is dimethylaminoethyl, diethylaminoethyl, methylethylaminoethyl, methyl-iso-butylaminoethyl, ethyl-iso-butylaminoethyl, methyl-tert-butylaminoethyl, or ethyl-tert-butylaminoethyl.

In certain embodiments,

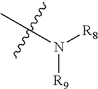

is:

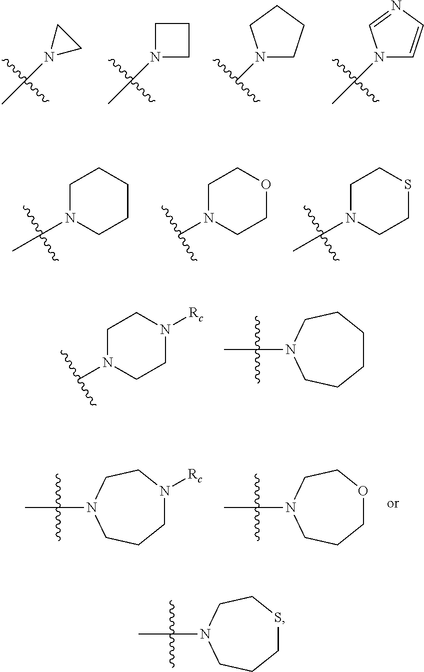

which R_{c} is H, Me, Et, n-Pr, i-Pr, n-Bu, i-Bu, t-Bu, CH₂CMe₃, Ph, or CH₂Ph.

In certain embodiments, R₅ is H. In certain other embodiments, R₅ is methyl. In yet other embodiments, R₅ is CH₂—S—(C₁-C₆)alky, e.g., CH₂—S—CH₃. In yet other embodiments, R₅ is CH₂—O—(C₁-C₆)alkyl, e.g., CH₂—O—CH₂—CH₃. In yet other embodiments, R₅ is (C₂-C₆)alkenyl, e.g., CH₂—CH═CH₂. In yet other embodiments, R₅ is benzyl. In yet other embodiments, R₅ is (C₂-C₆)OH. In yet other embodiments, R₅ is (C₁-C₆)-monoalkyl amine, e.g., CH₂—NH-Me. In yet other embodiments, R₅ is (C₁-C₆)-dialkyl amine, e.g., CH₂—CH₂—N(Et)₂. In yet other embodiments, R₅ is (C₁-C₆)-cyclic amine, e.g., CH₂—CH₂-morpholine.

In certain embodiments, R₅ is H, methyl, ethyl, n-propyl, i-propyl, n-butyl, i-butyl, t-butyl, CH₂CMe₃, phenyl, CH₂-phenyl,

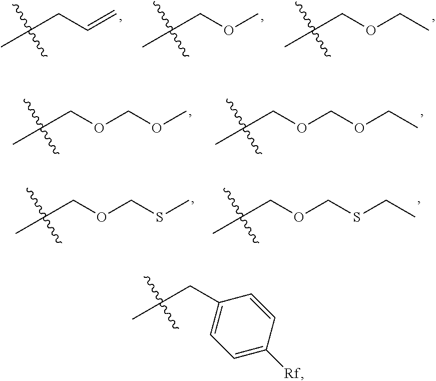

in which R_{f} is H, (C₁-C₄)alkyl or (C₁-C₄)alkoxy.

In certain embodiments, each occurrence R₈ and R₉ is independently H, (C₁-C₆)alkyl, phenyl, CH₂-phenyl, (C₁-C₆)alkyl-OH, (C₁-C₆)-alkyl-O—(C₁-C₆)alkyl, (C₁-C₆)alkyl-O—(CH₂)ₘOH, (C₁-C₆)alkyl-O—(CH₂)ₘ—O—(C₁-C₆alkyl), in which m is an integer of 1, 2, 3, 4 or 5. In certain other embodiments, m is an integer of 2, 3, or 4. In yet other embodiments, R₈ and R₉, together with the nitrogen atom to which they are attached

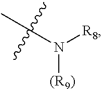

form a heterocycle selected from

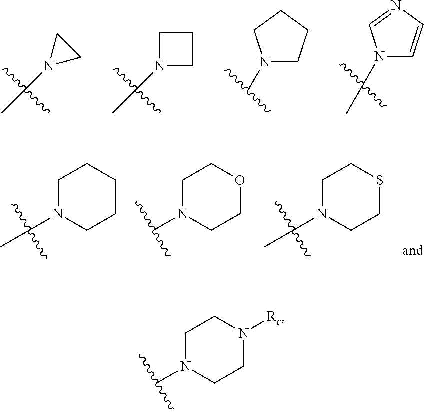

in which R_{c} is H, Me, Et, n-Pr, i-Pr, n-Bu, i-Bu, t-Bu, CH₂CMe₃, Ph, or CH₂Ph. In yet other embodiments, R₈ and R₉, together with the nitrogen atom to which they are attached

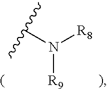

form a heterocycle selected from

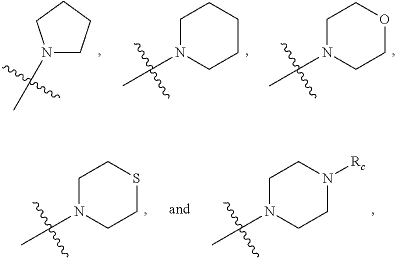

in which R_{c} is H, Me, Et, n-Pr, i-Pr, n-Bu, i-Bu, t-Bu, CH₂CMe₃, Ph, or CH₂Ph.

In certain embodiments, R₄ is

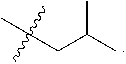 [SMILES: *CC(C)C]

In certain other embodiments, R₄ is

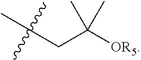

In yet other embodiments, R₄ is

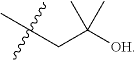 [SMILES: *CC(C)(C)O]

In yet other embodiments, R₄ is

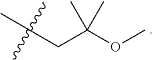 [SMILES: *CC(C)(C)OC]

In yet other embodiments, R₄ is

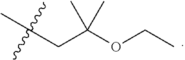 [SMILES: *CC(C)(C)OCC]

In yet other embodiments, R₄ is

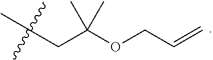 [SMILES: *CC(C)(C)OCC=C]

In yet other embodiments, R₄ is

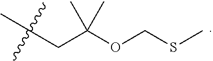 [SMILES: *CC(C)(C)OCSC]

In yet other embodiments, R₄ is

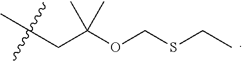 [SMILES: *CC(C)(C)OCSCC]

In yet other embodiments, R₄ is

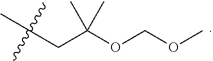 [SMILES: *CC(C)(C)OCOC]

In yet other embodiments, R₄ is

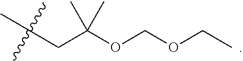 [SMILES: *CC(C)(C)OCOCC]

In yet other embodiments, R₄ is

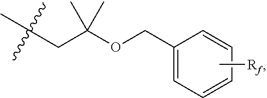

wherein R_{f} is H, (C₁-C₄)alkyl, or (C₁-C₄)alkoxy. In vet other embodiments, R₄ is

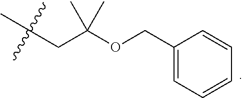 [SMILES: *CC(C)(C)OCc1ccccc1]

In yet other embodiments, R₄ is

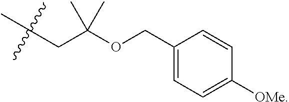 [SMILES: *CC(C)(C)OCc1ccc(OC)cc1]

In certain embodiments, R₅ is H, methyl, ethyl, n-propyl, i-propyl, n-butyl, i-butyl, t-butyl, CH₂CMe₃, phenyl, CH₂-phenyl,

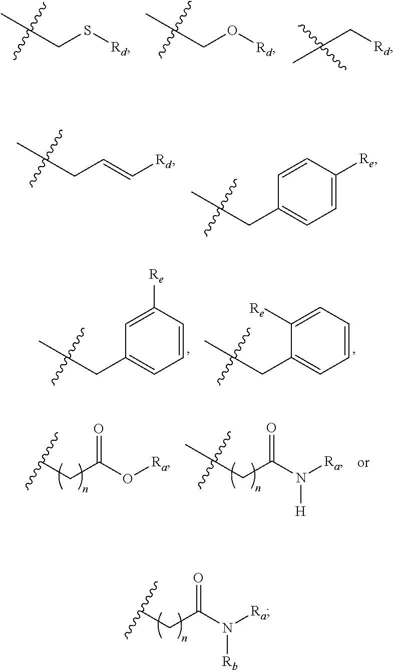

each R_{d} is independently Rₐ, ORₐ, CH₂ORₐ,

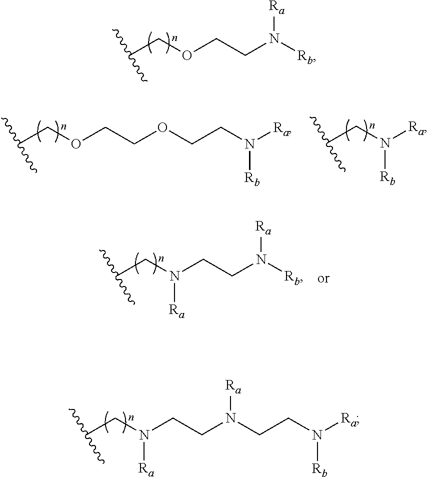

each Rₑ is independently H, Me, Et, ORₐ, CH₂ORₐ, CH₂CH₂ORₐ,

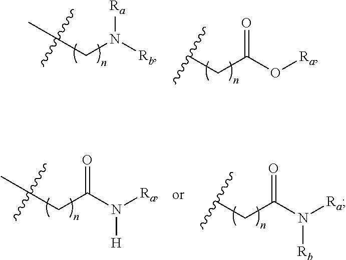

each of Rₐ and R_{b} is independently H, methyl, ethyl, n-propyl, i-propyl, n-butyl, i-butyl, t-butyl, CH₂CMe₃, phenyl, CH₂-phenyl,

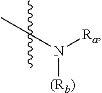

or Rₐ and R_{b}, together with the nitrogen atom to which they are attached

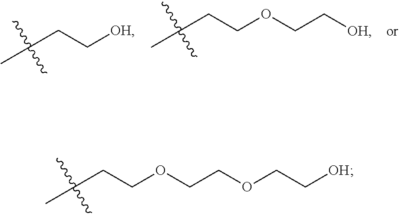

form a heterocycle selected from

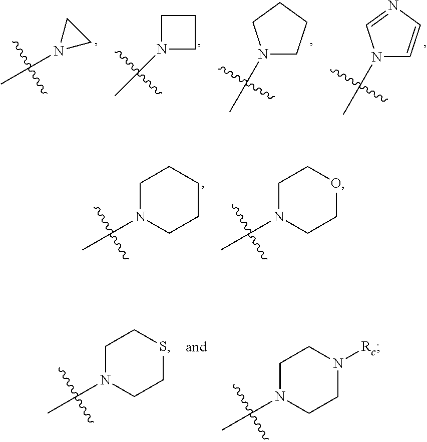

R_{c} is H, methyl, ethyl, n-propyl, i-propyl, n-butyl, i-butyl, t-butyl, CH₂CMe₃, phenyl, or CH₂-phenyl; and each n is independently 1, 2, 3, 4, 5 or 6.

In another aspect, the present invention provides a compound of formula (Ib):

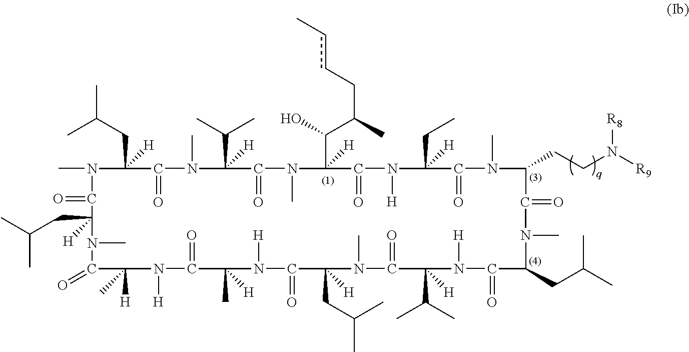

wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl,

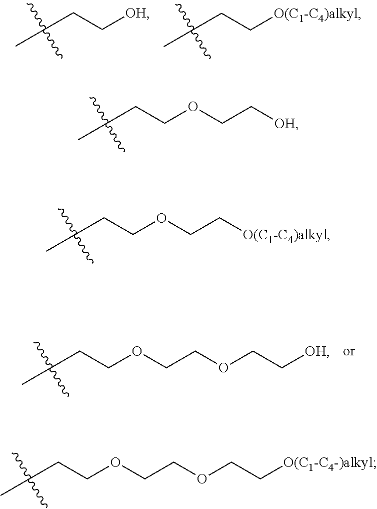

or R₈ and R₉, together with the nitrogen atom to which they are attached

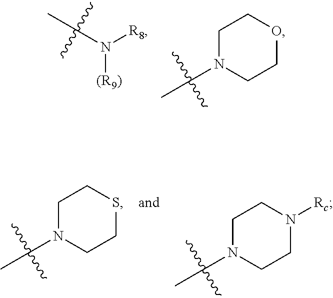

form a heterocycle selected from

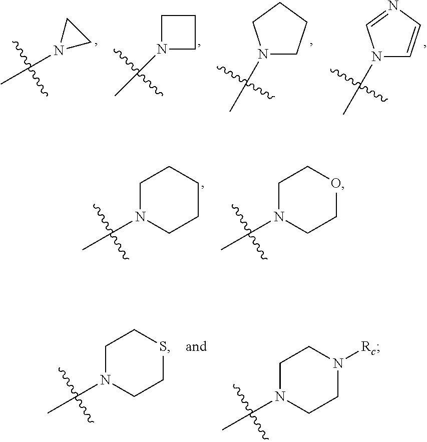

R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

In certain embodiments, each of R₈ and R₉ is independently H, methyl, ethyl, n-propyl, i-propyl, n-butyl, i-butyl, t-butyl, or CH₂CMe₃; or R₈ and R₉, together with the nitrogen atom to which they are attached

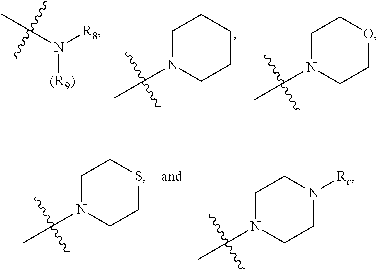

form a heterocycle selected from

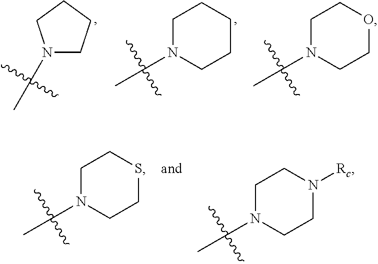

in which R_{c} is H, methyl, ethyl, n-propyl, i-propyl, n-butyl, i-butyl, t-butyl, CH₂CMe₃, phenyl, or CH₂-phenyl.

In certain embodiments, q is 1, 2, or 3.

In yet another aspect, the present invention provides a compound of formula (Ic):

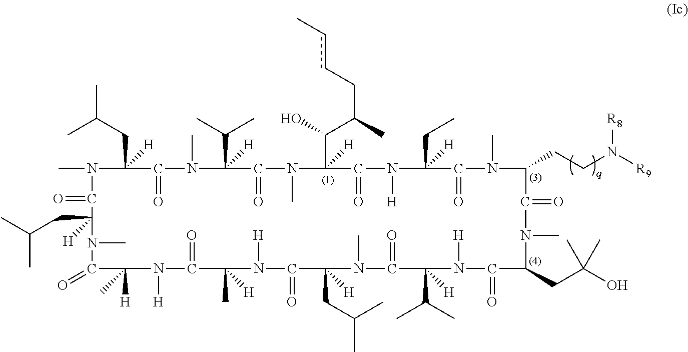

wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl,

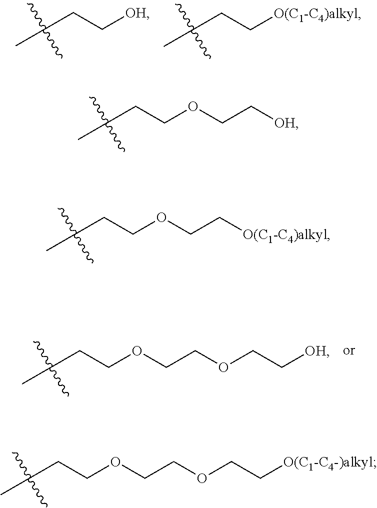

or R₈ and R₉, together with the nitrogen atom to which they are attached

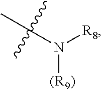

form a heterocycle selected from

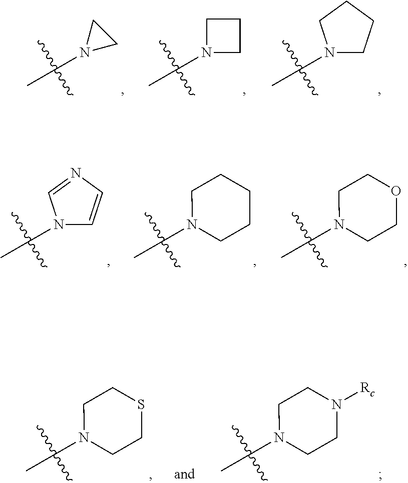

R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

In certain embodiments, each of R₈ and R₉ is independently H, methyl, ethyl, n-propyl, propyl, n-butyl, i-butyl, t-butyl, or CH₂CMe₃; or R₈ and R₉, together with the nitrogen atom to which they are attached

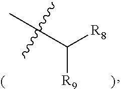

form a heterocycle selected from

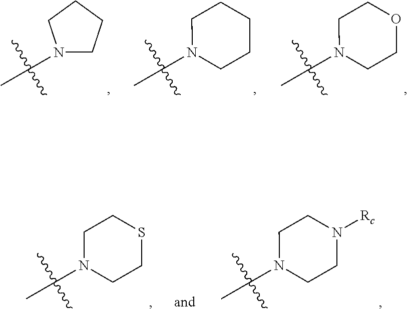

in which R_{c} is H, methyl, ethyl, n-propyl, i-propyl, n-butyl, i-butyl, t-butyl, CH₂CMe₃, phenyl, or CH₂-phenyl.

In certain embodiments, q is 1, 2, or 3.

In yet another aspect, the present invention provides a compound of formula (Id):

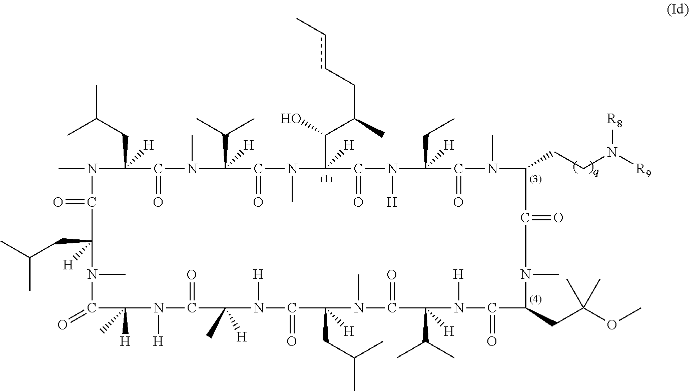

wherein ∥ represents a single bond or a double bond;
each of R₈ and R₉ is independently H, methyl, ethyl, n-propyl, propyl, n-butyl, i-butyl, t-butyl, CH₂CMe₃, phenyl, CH₂-phenyl,

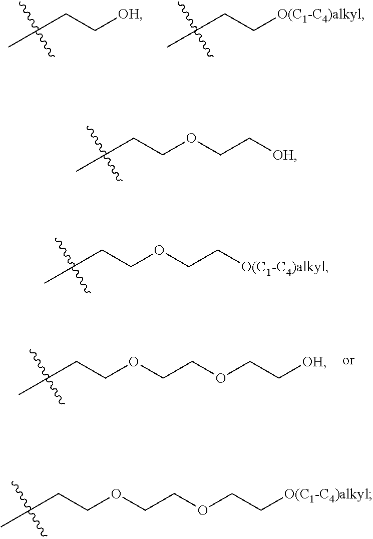

or R₈ and R₉, together with the nitrogen atom to which they are attached

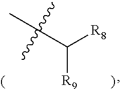

form a heterocycle selected from

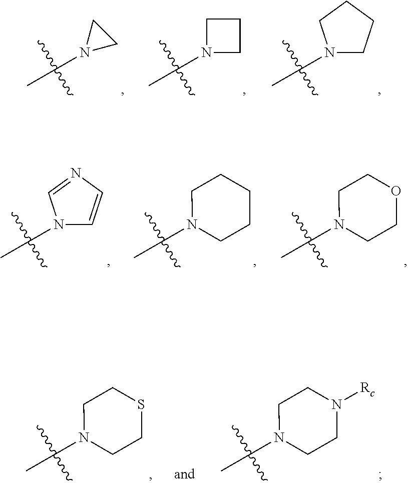

R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

In certain embodiments, each of R₈ and R₉ is independently H, methyl, ethyl, n-propyl, propyl, n-butyl, i-butyl, t-butyl, or CH₂CMe₃; or R₈ and R₉, together with the nitrogen atom to which they are attached

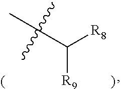

form a heterocycle selected from

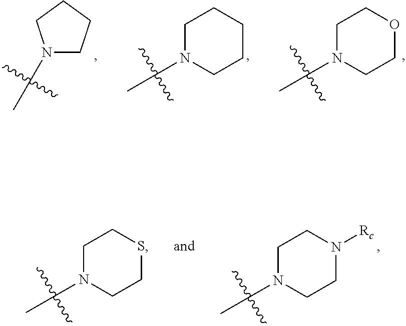

in which Rₑ is H, methyl, ethyl, n-propyl, i-propyl, n-butyl, i-butyl, t-butyl, CH₂CMe₃, phenyl, or CH₂-phenyl.

In certain embodiments, q is 1, 2, or 3.

In yet another aspect, the present invention provides a compound of formula (Ie):

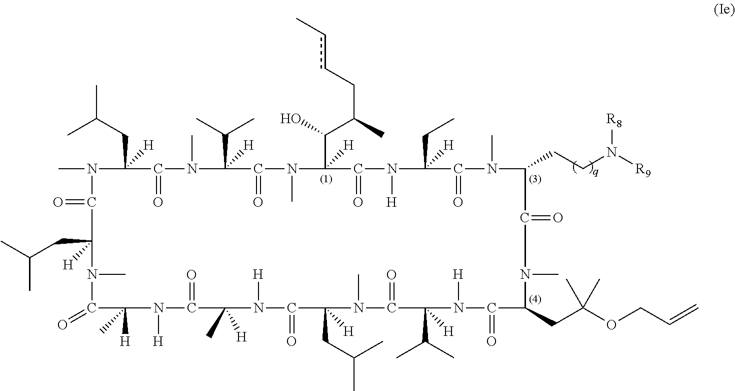

wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl,

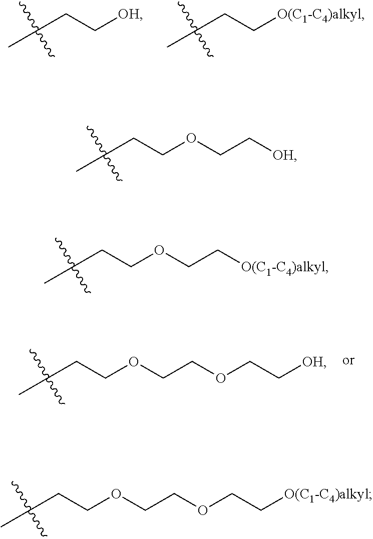

or R₈ and R₉, together with the nitrogen atom to which they are attached

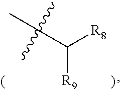

form a heterocycle selected from

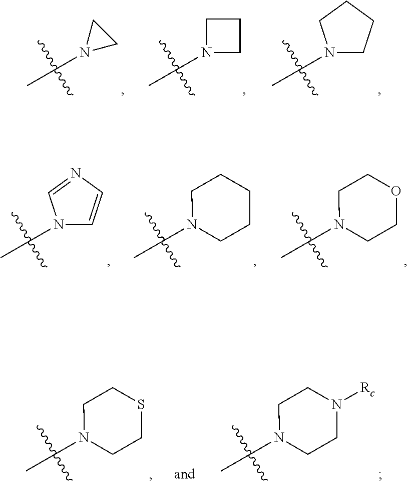

R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

In certain embodiments, each of R₈ and R₉ is independently H, methyl, ethyl, n-propyl, i-propyl, n-butyl, i-butyl, t-butyl, or CH₂CMe₃; or R₈ and R₉, together with the nitrogen atom to which they are attached

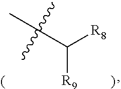

form a heterocycle selected from

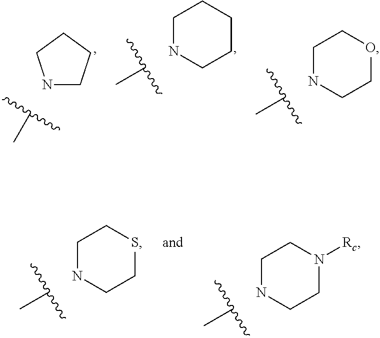

in which R_{c} is H, methyl, ethyl, n-propyl, i-propyl, n-butyl, i-butyl, t-butyl, CH₂CMe₃, phenyl, or CH₂-phenyl.

In certain embodiments, q is 1, 2, or 3.

In certain embodiments, R₄ is

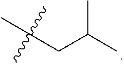 [SMILES: *CC(C)C]

In certain other embodiments, R₄ is

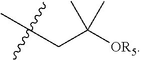

In yet other embodiments, R₄ is

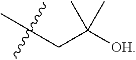 [SMILES: *CC(C)(C)O]

In yet other embodiments, R₄ is

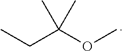

In yet other embodiments, R₄ is

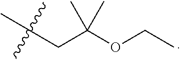 [SMILES: *CC(C)(C)OCC]

In yet other embodiments, R₄ is

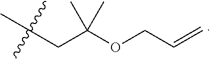 [SMILES: *CC(C)(C)OCC=C]

In yet other embodiments, R₄ is

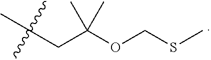 [SMILES: *CC(C)(C)OCSC]

In yet other embodiments, R₄ is

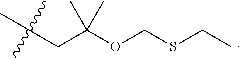 [SMILES: *CC(C)(C)OCSCC]

In yet other embodiments, R₄ is

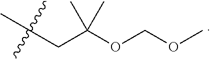 [SMILES: *CC(C)(C)OCOC]

In yet other embodiments, R₄ is

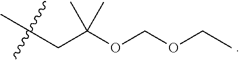 [SMILES: *CC(C)(C)OCOCC]

In certain embodiments, R₄ is

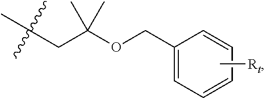

wherein R_{f} is H, (C₁-C₄)alkyl, or (C₁-C₄)alkoxy. In certain other embodiments, R_{f} is at para-position. In yet other embodiments, R₄ is

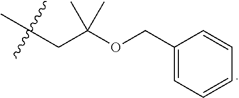 [SMILES: *CC(C)(C)OCc1ccccc1]

In yet other embodiments, R₄ is

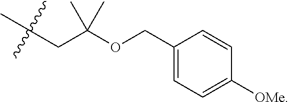 [SMILES: *CC(C)(C)OCc1ccc(OC)cc1]

In one aspect, the present invention provides a compound as described in the Examples.

In another aspect, the present invention provides a pharmaceutical composition comprising at least one compound described herein and a pharmaceutically-acceptable carrier or diluent.

In a further aspect, the present invention provides a method for treating or preventing a viral infection in a mammalian species in need thereof, the method comprising administering to the mammalian species a therapeutically effective amount of at least one compound described herein. In certain embodiments, the viral infection is HIV infection. In certain other embodiments, the viral infection is HBV infection. In yet other embodiments, the viral infection is HCV infection. In yet other embodiments, the viral infection is influenza A virus infection, severe acute respiratory syndrome coronavirus infection or vaccinia virus infection. In yet other embodiments, the viral infection is herpes simplex virus.

In another aspect, the present invention provides a method for treating or preventing hepatitis C virus infection or hepatitis B virus infection in a mammalian species in need thereof, the method comprising administering to the mammalian species a therapeutically effective amount of at least one compound described herein.

In another aspect, the present invention provides a method for treating or preventing a central nervous system disorder in a mammalian species in need thereof, the method comprising administering to the mammalian species a therapeutically effective amount of at least one compound described herein. In certain embodiments, the central nervous system disorder is mitochondrial protection for stroke, traumatic brain and spinal cord injury, Alzheimer, Parkinson's, or Huntington's Diseases.

In yet another aspect, the present invention provides a method for treating or preventing a cardiovascular disease in a mammalian species in need thereof, the method comprising administering to the mammalian species a therapeutically effective amount of at least one compound described herein. In certain embodiments, the cardiovascular disease is reperfusion injury, heart attack, or chronic heart failure.

In yet another aspect, the present invention provides a method for treating or preventing an inflammation disease in a mammalian species in need thereof, the method comprising administering to the mammalian species a therapeutically effective amount of at least one compound described herein. In certain embodiments, the inflammation disease is respiratory inflammation, asthma, ulcerative colitis, rheumatoid arthritis, or dry eye disease.

In a further aspect, the present invention provides a method for preparing a compound of formula (II),

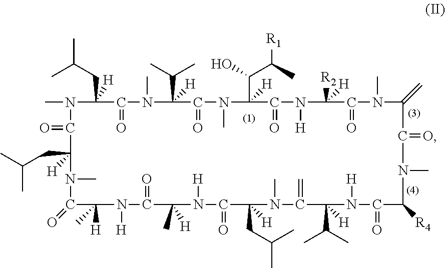

wherein
R₁ is n-butyl or (E)-but-2-enyl;
R₂ is ethyl, 1-hydroxyethyl, isopropyl or n-propyl;
R₄ is

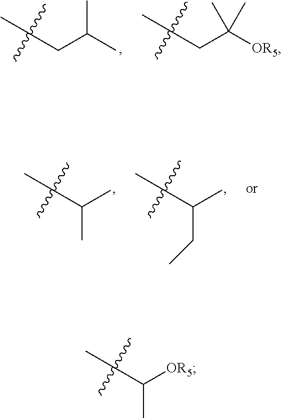

R₅ is:
   H;
   (C₁-C₆)alkyl, optionally substituted by one or more groups R₇ which may be the same or different;
   (C₂-C₆)alkenyl, optionally substituted by one or more groups which may be the same or different selected from hydroxy, (C₁-C₆)alkyl, aryl (e.g., phenyl), (CH₂)ₚOR_{A}, O(CH₂)ₘOH, O(CH₂)ₘO(CH₂)ₘOH, O(CH₂)ₘNR_{A}R_{B}, O(CH₂)ₘO(CH₂)ₘNR_{A}R_{B}, (CH₂)ₚNR_{A}R_{B}, (CH₂)ₚNR_{c}(CH₂)ₘNR_{A}R_{B}, (CH₂)ₚNR_{C}(CH₂)ₘNR_{C}(CH₂)ₘNR_{A}R_{B}, (CH₂)ₚC(═O)NR_{A}R_{B}, (CH₂)ₚC(═O)OR_{A};
   (C₂-C₆)alkynyl, optionally substituted by one or one or more groups which may be the same or different selected from halogen, hydroxy, amino, monoalkylamino and dialkylamino;
   (C₃-C₇)cycloalkyl, optionally substituted by one or more groups which may be the same or different selected from halogen, hydroxy, amino, monoalkylamino and dialkylamino;
   phenyl or CH₂-phenyl, optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B}, (CH₂)ₚC(═O)OR_{A};
each occurrence of R₇ is independently halogen, hydroxy, aryl (e.g., phenyl), S(C₁-C₆)alkyl, SR_{A}, OR_{A}, O(CH₂)ₘOH, O(CH₂)ₘO(CH₂)ₘOH, O(CH₂)ₘO(C₁-C₆)alkyl, O(CH₂)ₘO(CH₂)ₘO(C₁-C₆)alkyl, C(═O)OR_{A}, C(═O)NR_{A}R_{B}, NR_{A}R_{B}, O(CH₂)ₘNR_{A}R_{B}, O(CH₂)ₘO(CH₂)ₘNR_{A}R_{B}, NR_{C}(CH₂)ₘNR_{A}R_{B}, or NR_{c}(CH₂)ₘNR_{c}(CH₂)ₘNR_{A}R_{B}, wherein said aryl or phenyl is optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B} and (CH₂)ₚC(═O)OR_{A};
comprising contacting a compound of formula (III),

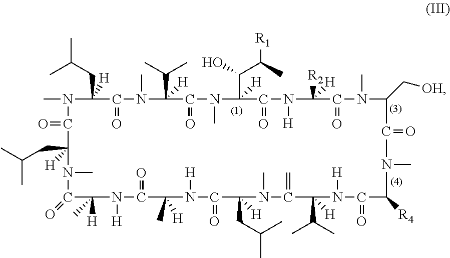

wherein R₁, R₂ and R₄ are defined as hereinabove,
with a reagent selected from: (1) MsCl or TsCl; (2) CBr₄/PPh₃; and (3)

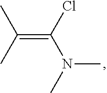

to provide the compound of formula (II).

In certain embodiments, R₄ is

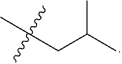 [SMILES: *CC(C)C]

In certain other embodiments, R₄ is

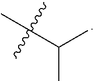 [SMILES: *C(C)C]

In yet other embodiments, R₄ is

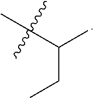 [SMILES: *C(C)CC]

In yet other embodiments, R₄ is

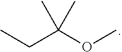

In yet other embodiments, R₄ is

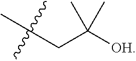 [SMILES: *CC(C)(C)O]

In yet other embodiments, R₄ is

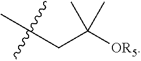

In yet other embodiments, R₄ is

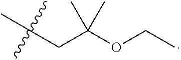 [SMILES: *CC(C)(C)OCC]

In yet other embodiments, R₄ is

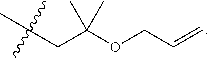 [SMILES: *CC(C)(C)OCC=C]

In yet other embodiments, R₄ is

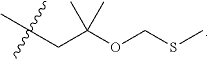 [SMILES: *CC(C)(C)OCSC]

In yet other embodiments, R₄ is

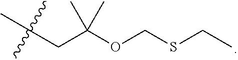 [SMILES: *CC(C)(C)OCSCC]

In yet other embodiments, R₄ is

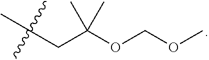 [SMILES: *CC(C)(C)OCOC]

In yet other embodiments, R₄ is

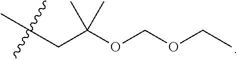 [SMILES: *CC(C)(C)OCOCC]

### Methods of Preparation

In certain embodiments, the compound of formula (I) can be prepared by treating cyclosporin A or an analog thereof with a base (e.g., LDA) to form a sarcosine enolate at position 3. CO₂ gas is then introduced to yield carboxylic acid-3-cyclosporin, which can be converted to its corresponding methyl ester. The reduction of the methyl ester side chain converts the compound to its corresponding alcohol. Then its mesylate, or tosylate, or chloride can be formed by treatment with MsCl or TsCl in dichloromethane solution. The methylene on the sarcosine can be produced by treatment of its mesylate, or tosylate, or chloride with a base (e.g., NaH). In certain embodiments, an alkyl chain with different functional groups can be introduced on the sarcosine of position 3 through a 1,4-addition reaction on the methylene group. For example:

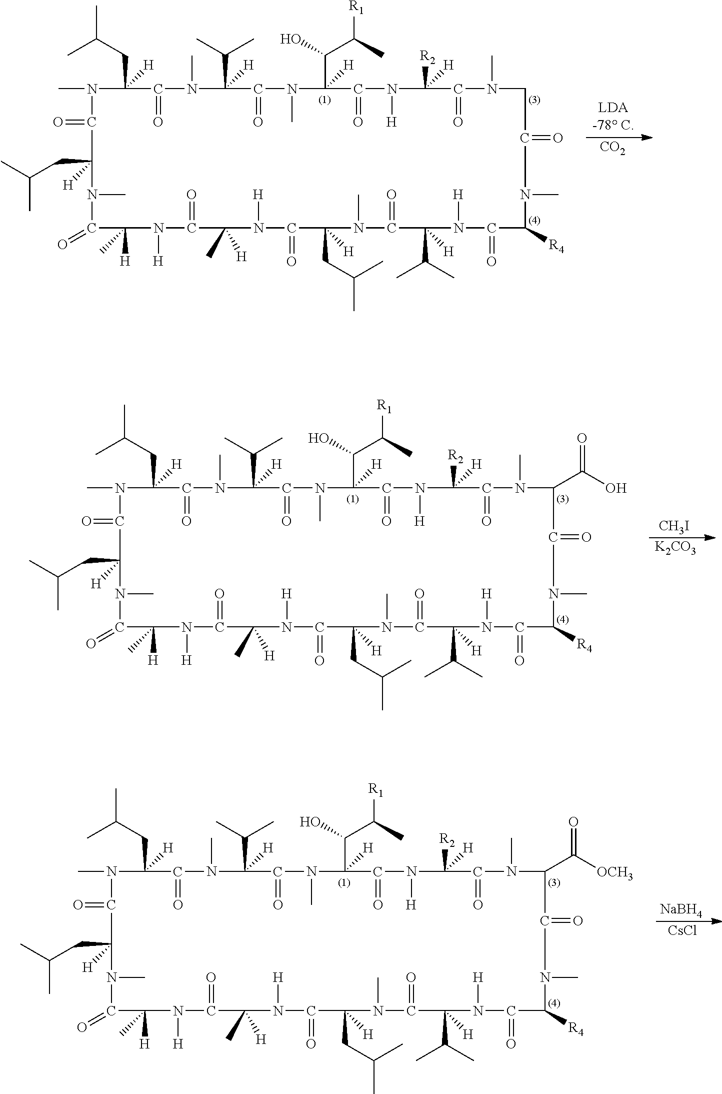

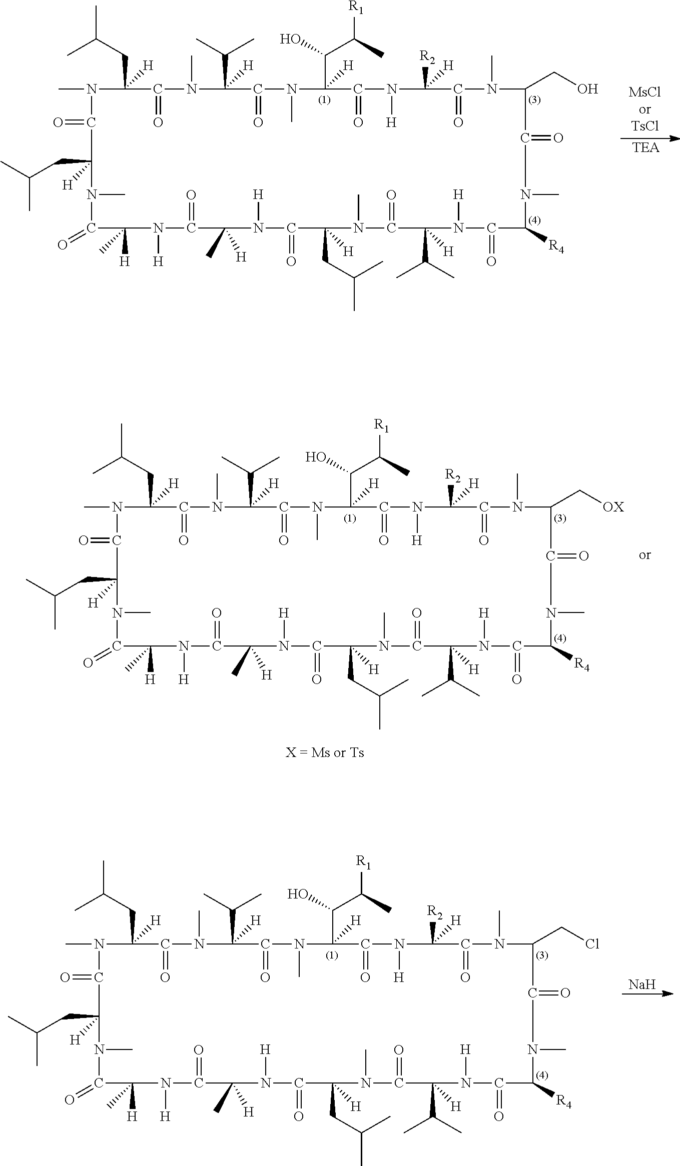

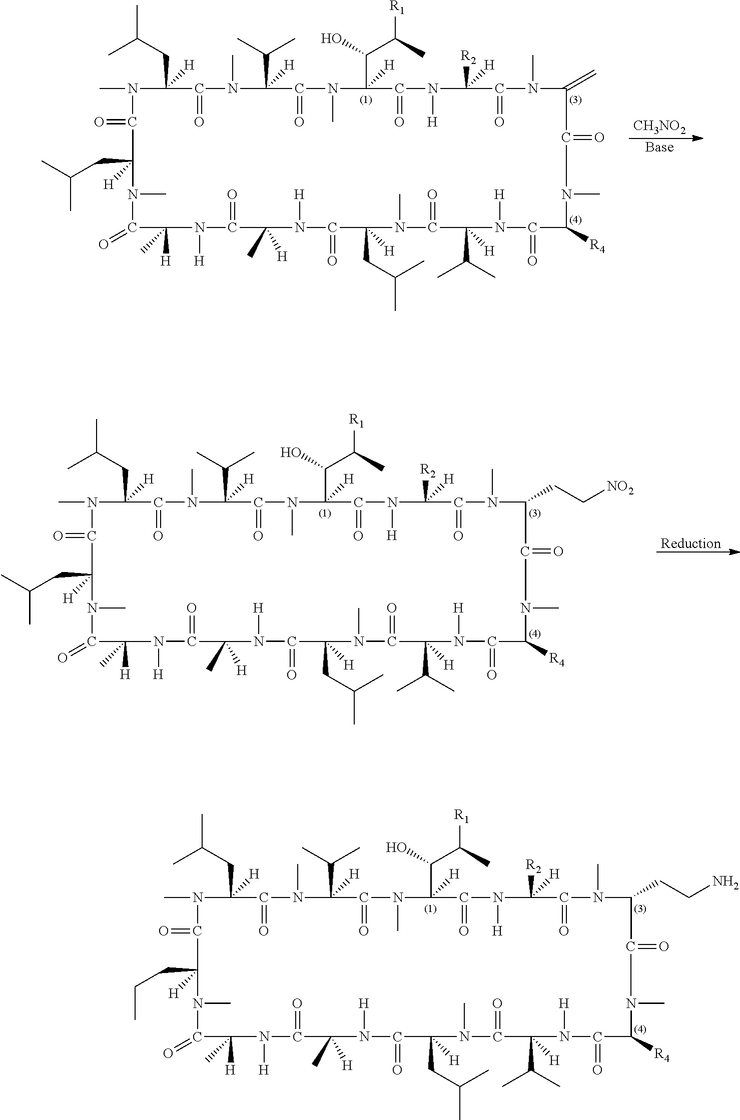

In certain other embodiments, the resulting primary amine above can be alkylated or transformed into various amine derivatives. In certain other embodiments, when R₄ is

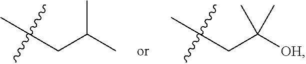

it can be further converted to

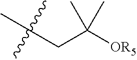

through alkylation.

The double bond hydrogenation of MeBmt at position 1 of cyclosporine can provide (Dihydro-MeBmt)-1-cyclosporin by using a method described by U.S. Pat. Nos. 4,108,985, 5,767,069, and 5,981,479, each of which is incorporated herein by reference.

[(γ-Hydroxy)-NMeLeu]-4-cyclosporin (structure shown below) can be prepared by Sebekia benihana biotransformation according to a method described by Kuhnt M. et al., 1996, Microbial Biotransformation Products of Cyclosporin A, J. Antibiotics, 49 (8), 781, incorporated herein by reference in its entirety.

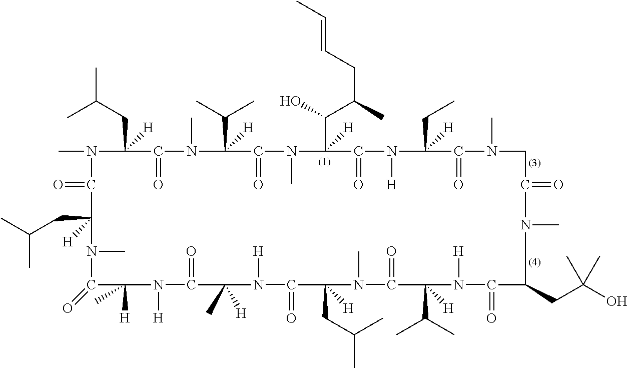 [SMILES: C/C=C/C[C@@H](C)[C@@H](O)[C@H]1C(=O)N[C@@H](CC)C(=O)N(C)CC(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

### Pharmaceutical Compositions

This invention also provides a pharmaceutical composition comprising at least one of the compounds as described herein or a pharmaceutically-acceptable salt or solvate thereof, and a pharmaceutically-acceptable carrier.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As set out above, certain embodiments of the present pharmaceutical agents may be provided in the form of pharmaceutically-acceptable salts. The term “pharmaceutically-acceptable salt”, in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, butionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate, magnesium stearate, and polyethylene oxide-polybutylene oxide copolymer as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient, which can be combined with a carrier material to produce a single dosage form will generally be that amount of the compound which produces a therapeutic effect. Generally, out of 100%, this amount will range from about 1% to about 99% of active ingredient, preferably from about 5% to about 70%, most preferably from about 10% to about 30%.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polybutylene oxide copolymer; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxybutylmethyl cellulose), lubricant, inert diluent, preservative, disintegrant (for example, sodium starch glycolate or cross-linked sodium carboxymethyl cellulose), surface-active or dispersing agent. Molded tablets, may be, made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxybutylmethyl cellulose in varying butortions to provide the desired release profile, other polymer matrices, liposomes and/or microspheres. They may be sterilized by, for example, filtration through a bacteria-retaining filter, or by incorporating sterilizing agents in the form of sterile solid compositions, which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions, which can be used include polymeric substances and waxes. The active ingredient can also be in micro-encapsulated form, if apbutriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isobutyl alcohol, ethyl carbonate, ethyl acetate, benzyl alcohol, benzyl benzoate, butylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Additionally, cyclodextrins, e.g., hydroxybutyl-.beta.-cyclodextrin, may be used to solubilize compounds.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active pharmaceutical agents of the invention.

Formulations of the present invention which are suitable for vaginal administration also include pessaries, tampons, creams, gels, pastes, foams or spray formulations containing such carriers as are known in the art to be apbutriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or butellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary butellants, such as chlorofluorohydrocarbons and volatile unsubstituted hydrocarbons, such as butane and butane.

Transdermal patches have the added advantage of providing controlled delivery of a compound of the present invention to the body. Such dosage forms can be made by dissolving, or dispersing the pharmaceutical agents in the buter medium. Absorption enhancers can also be used to increase the flux of the pharmaceutical agents of the invention across the skin. The rate of such flux can be controlled, by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally-administered drug form is accomplished by dissolving or suspending the drug in an oil vehicle. One strategy for depot injections includes the use of polyethylene oxide-polybutylene oxide copolymers wherein the vehicle is fluid at room temperature and solidifies at body temperature.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly (orthoesters) and poly (anhydrides). Depot injectable formulations are also prepared by entrapping the drug in liposomes or microemulsions, which are compatible with body tissue.

When the compounds of the present invention are administered as pharmaceuticals, to humans and animals, they can be given per se or as a pharmaceutical composition containing, for example, 0.1% to 99.5% (more preferably, 0.5% to 90%) of active ingredient in combination with a pharmaceutically acceptable carrier.

The compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another anti-HCV agent), or they may achieve different effects (e.g., control of any adverse effects).

The compounds of the invention may be administered intravenously, intramuscularly, intraperitoneally, subcutaneously, topically, orally, or by other acceptable means. The compounds may be used to treat arthritic conditions in mammals (i.e., humans, livestock, and domestic animals), birds, lizards, and any other organism, which can tolerate the compounds.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art. The following examples contain important additional information, exemplification and guidance which can be adapted to the practice of this invention in its various embodiments and equivalents thereof.

### EXAMPLES

### Example 1

### [α-Methylene-Sar]-3-cyclosporin

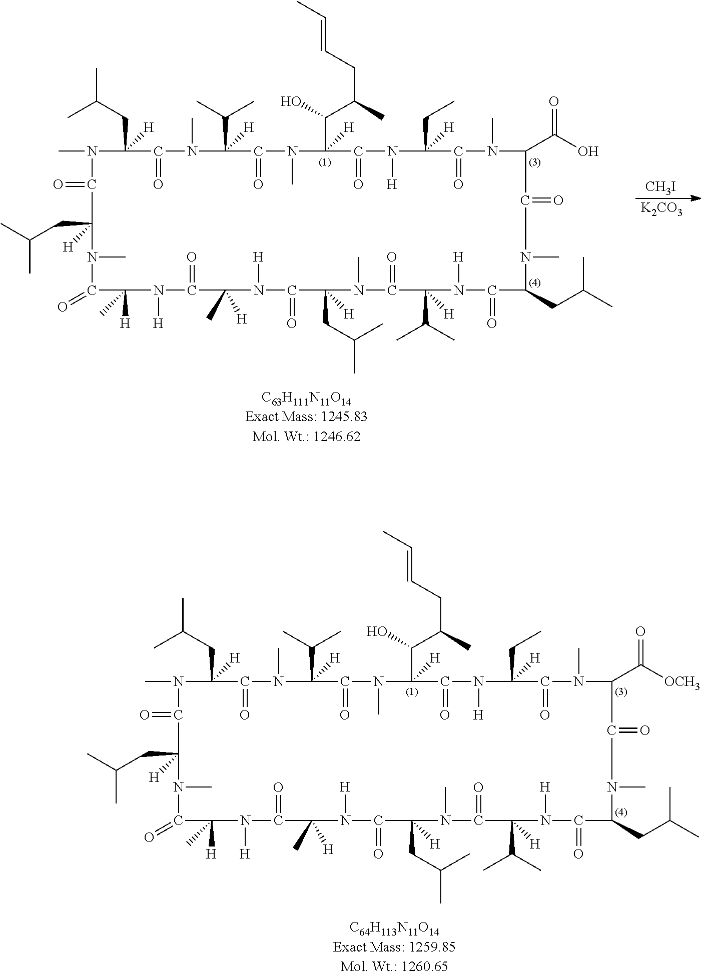

[α-Carboxy-sar]-3-cyclosporin (5.00 g, 4.01 mmol) was dissolved in N,N-dimethylformamide (30 ml). Iodomethane (2.85 g, 20.10 mmol) and potassium carbonate (1.38 g, 10.00 mmol) were added. The mixture was stirred at room temperature for 2 hours. Then ethyl acetate (60 ml) and water (60 ml) were added and the mixture was separated. The ethyl acetate layer was washed with brine, dried over magnesium sulfate and evaporated under reduced pressure to give 5.32 g of crude product of [α-methoxycarbonyl-Sar]-3-cyclosporin, which was directly used for the next step without purification (yield: ˜100%) [Molecular Formula: C₆₄H₁₁₃N₁₁O₁₄; Exact Mass: 1259.85; MS (m/z): 1260.7 (M+1)⁺, 1282.7 (M+Na)⁺; TLC R_{f}: 0.55 (dichloromethane/methanol=9/1)].

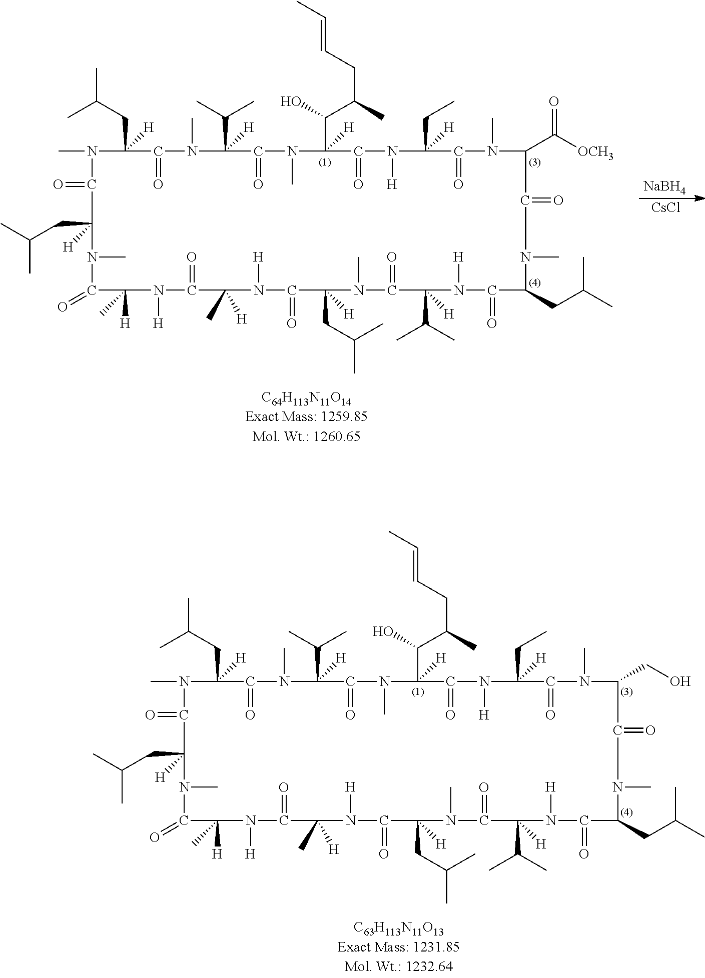

[α-Methoxycarbonyl-Sar]-3-cyclosporin (2.00 g, 1.59 mmol) was dissolved in tetrahydrofuran (30 ml). Cesium chloride (1.33 g, 7.90 mmol) and sodium borohydride (0.60 g, 15.89 mmol) were added in portions. Then methanol (30 ml) was added dropwise to the mixture over 2 hours. After addition, the mixture was stirred at room temperature overnight. Most of solvent was then evaporated under reduced pressure. Ethyl acetate (50 ml) and water (50 ml) were added. The ethyl acetate layer was separated, washed with brine, dried over magnesium sulfate and evaporated under reduced pressure to give 1.99 g of crude product of [(R)-α-hydroxymethyl-Sar]-3-cyclosporin, which was purified by on silica gel column with dichloromethane/methanol (from 100:0 to 95:5) to give the 1.50 g of pure product (yield: 76%) [Molecular Formula: C₆₃H₁₁₃N₁₁O₁₃; Exact Mass: 1231.85; MS (m/z): 1232.7 (M+1)⁺, 1254.7 (M+Na)⁺].

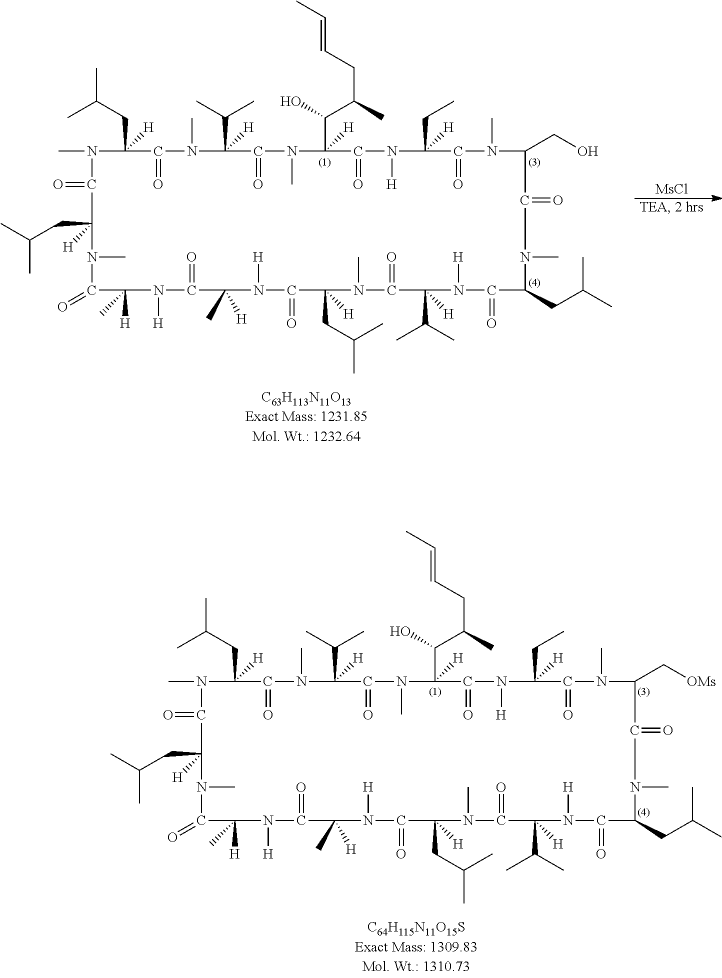

To a solution of [α-hydroxymethyl-Sar]-3-cyclosporin (30 mg, 0.024 mmol) in dichloromethane (2 ml) at 0° C. were added triethylamine (52.8 μl, d 0.726, 0.38 mmol), and methanesulfonyl chloride (15.6 μl, d 1.477, 0.20 mmol). After stirred at room temperature for two hours, the reaction mixture was washed with brine, dried over magnesium sulfate and evaporated under reduced pressure to give 33 mg of crude product of [α-methanesulfonoylmethyl-Sar]-3-cyclosporin, which was directly used in next step reaction without further purification [Molecular Formula: C₆₄H₁₄₅N₁₁O₁₅S; Exact Mass: 1309.83; MS (m/z): 1310.7 (M+1)⁺].

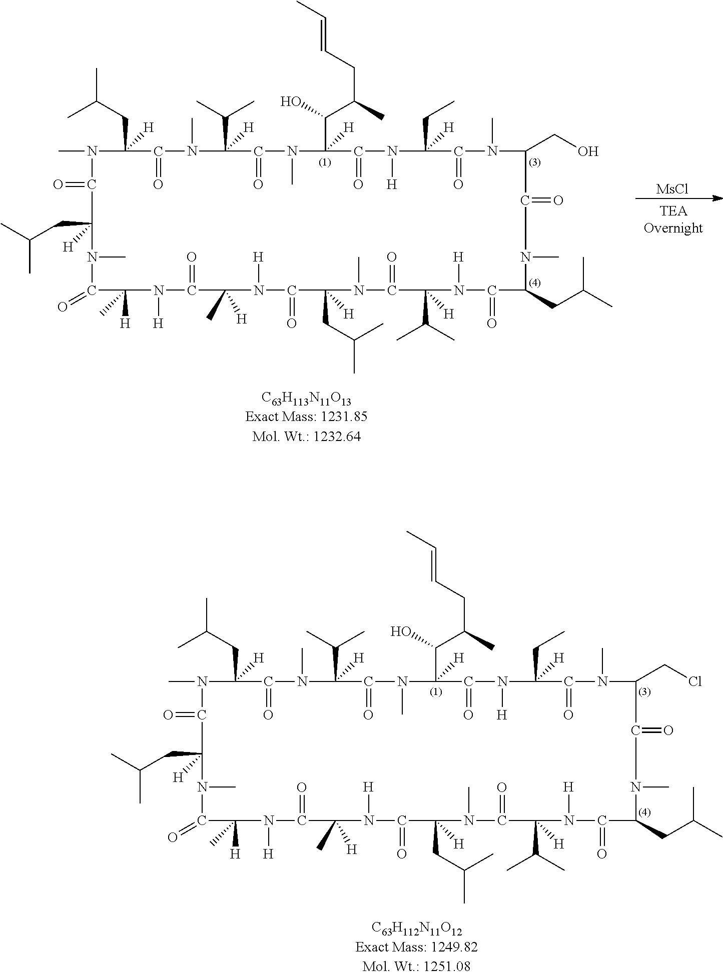

To a solution of [α-hydroxymethyl-Sar]-3-cyclosporin (30 mg, 0.024 mmol) in dichloromethane (2 ml) at 0° C. were added triethylamine (52.8 μL, 0.384 mmol, 16 equivalents) and methanesulfonyl chloride (23 mg, 0.20 mmol). After stirred at room temperature overnight, the reaction mixture was washed with brine, dried over magnesium sulfate and evaporated under reduced pressure to give 30 mg of crude product of [α-chloromethyl-Sar]-3-cyclosporin, which was directly used in next step reaction without further purification [Molecular Formula: C₆₃H₁₁₂ClN₁₁O₁₂; Exact Mass: 1249.82; MS (m/z): 1250.7 (M+1)⁺, 1272.9 (M+Na)⁺].

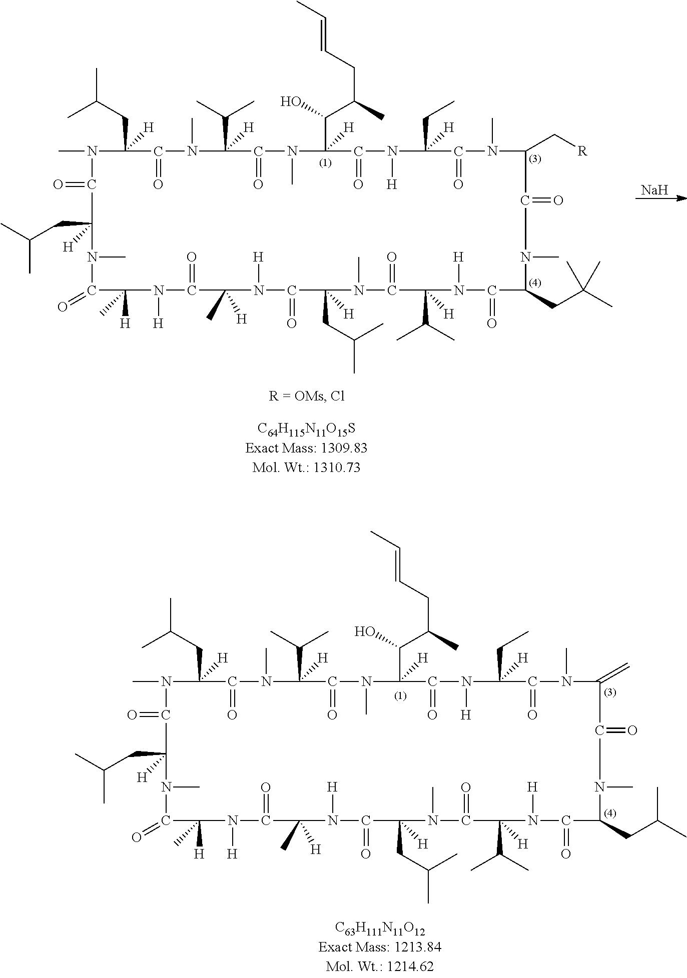

To a solution of either [α-methanesulfonatemethyl-Sar]-3-cyclosporin (33 mg, 0.025 mmol) or [α-chloromethyl-Sar]-3-cyclosporin (30 mg, 0.025 mmol) or the mixture of both in tetrahydrofuran (3 ml) was added sodium hydride (15.3 mg, 60% in oil, 0.38 mmol, 10 equivalents) at 0° C. The mixture was stirred at 0° C. for one hour and then warmed up to room temperature for 30 minutes. After removal of solvent, the residue was dissolved in dichloromethane (20 ml). The dichloromethane layer was washed with 1 N hydrochloric acid, saturated sodium bicarbonate solution and brine, dried over magnesium sulfate and evaporated under reduced pressure. The residue was purified by chromatography on silica gel using dichloromethylene/methanol (20/1) to give 16 mg of product of [α-methylene-Sar]-3-cyclosporin (yield: 54%) [Molecular Formula: C₆₃H₁₁₁N₁₁O₁₂; Exact Mass: 1213.84; MS (m/z): 1214.7 (M+1)⁺, 1236.7 (M+Na)⁺; TLC R_{f}: 0.55 (ethyl acetate/methanol=20/1); HPLC RT: 7.0 min. (C8 reverse phase column: 150 mm; acetonitrile/water (0.05% trifluoroacetic acid); operation temperature: 64° C.; detector: 210 nm)].

### Example 2

### [α-Methylene-Sar]-3-[(γ-hydroxy)-NMeLeu]-4-cyclosporin

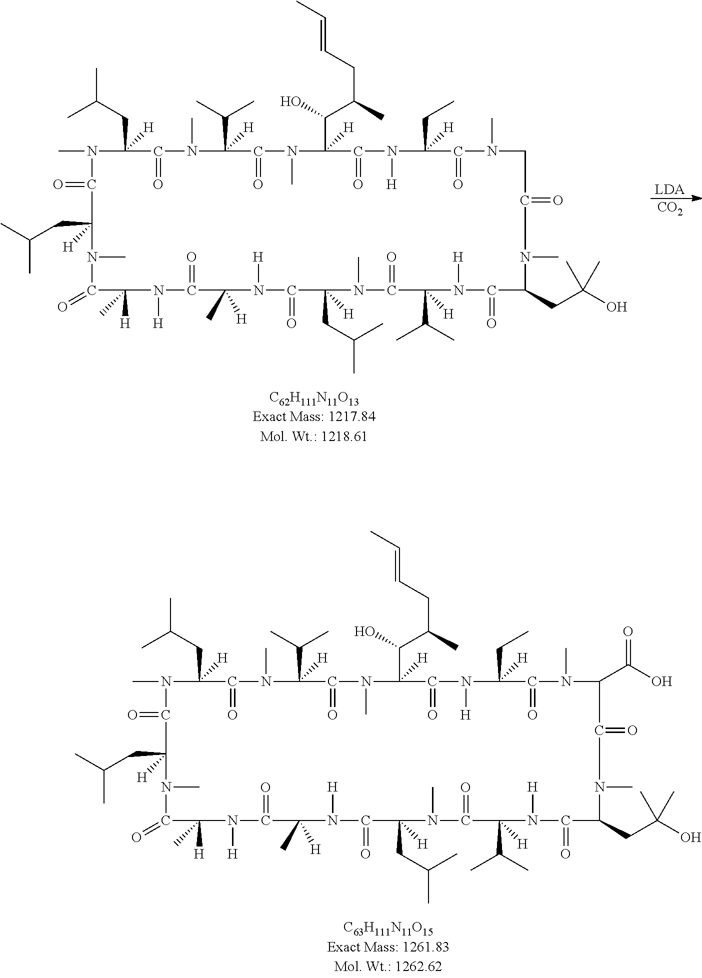 [SMILES: C/C=C/C[C@@H](C)[C@@H](O)[C@H]1C(=O)N[C@@H](CC)C(=O)N(C)CC(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; "CO2" ; "LDA" ; C/C=C/C[C@@H](C)[C@@H](O)[C@H]1C(=O)N[C@@H](CC)C(=O)N(C)C(C(=O)O)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

To a solution of lithium diisopropylamide (2.0 M in tetrahydrofuran, 23 ml, 46 mmol) in tetrahydrofuran (80 ml) at −78° C. under nitrogen, [(γ-hydroxy)-NMeLeu]-4-cyclosporin (4.40 g, 3.61 mmol) in tetrahydrofuran (15 ml) was added over 3 minutes. After the mixture was stirred at −78° C. for 3 hours, carbon dioxide gas was bubbled into the reaction mixture for one hour. Then the mixture was allowed to warm to room temperature slowly and kept stirring for 3 hours. Most of tetrahydrofuran was evaporated. Dichloromethane (100 ml) and water (50 ml) were added. The PH of the mixture was adjusted to around 5 by adding aqueous citric acid solution. The mixture was separated and the organic layer was washed with brine, dried over magnesium sulfate and evaporated under reduced pressure to give 3.20 g of crude product of [α-carboxy-Sar]-3-[(γ-hydroxy)-NMeLeu]-4-cyclosporin, which was used for next step without purification [Molecular Formula: C₆₃H₁₁₁N₁₁O₁₅; Exact Mass: 1261.83; MS (m/z): 1262.49 (M+1)⁺].

[(γ-Hydroxy)-NMeLeu]-4-cyclosporin was prepared by Sebekia benihana biotransformation according to a method described by Kuhnt M. et al., 1996, Microbial Biotransformation Products of Cyclosporin A, J. Antibiotics, 49 (8), 781.

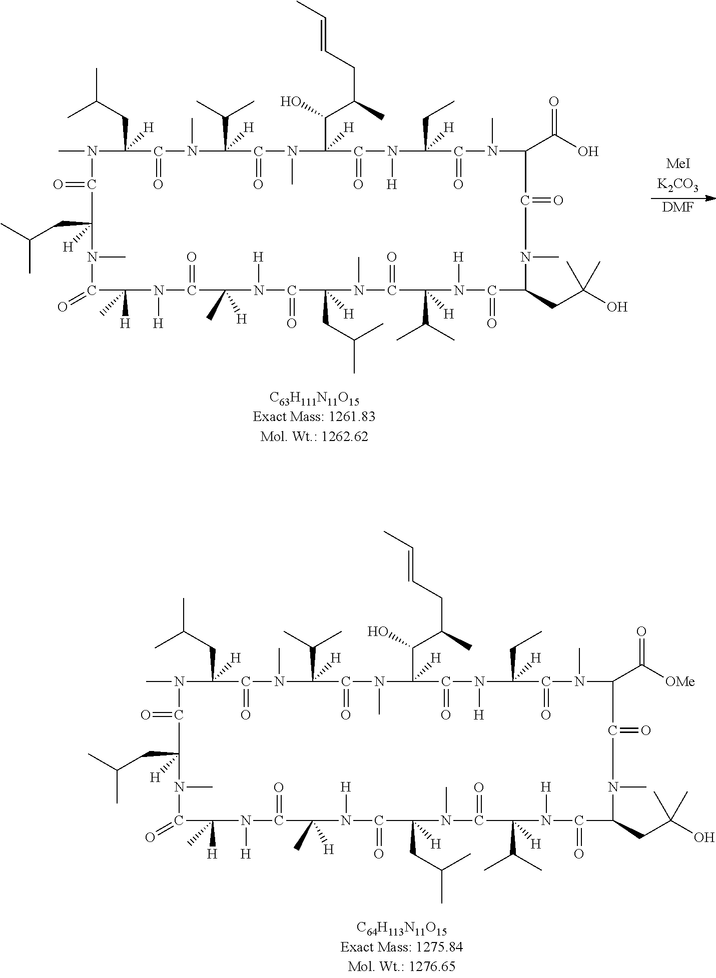

To a mixture of [α-carboxy-Sar]-3-[(γ-hydroxy)-NMeLeu]-4-cyclosporin (3.20 g 2.53 mmol) and potassium carbonate (1.30 g, 9.40 mmol) in N,N-dimethylformamide (20 ml) was added iodomethane (1.80 g, 12.70 mmol). The mixture was stirred overnight at room temperature. Dichloromethane (80 ml) and water (50 ml) were added and the mixture was separated. The dichloromethane layer was washed with water (25 ml) and brine (25 ml), dried over magnesium sulfate and evaporated under reduced pressure to give crude 3.00 g of product of [α-methoxycarbonyl-Sar]-3-[(γ-hydroxy)-NMeLeu]-4-cyclosporin [Molecular Formula: C₆₄H₁₁₃N₁₁O₁₅; Exact Mass: 1275.84; MS (m/z): 1276.75 (M+1)⁺].

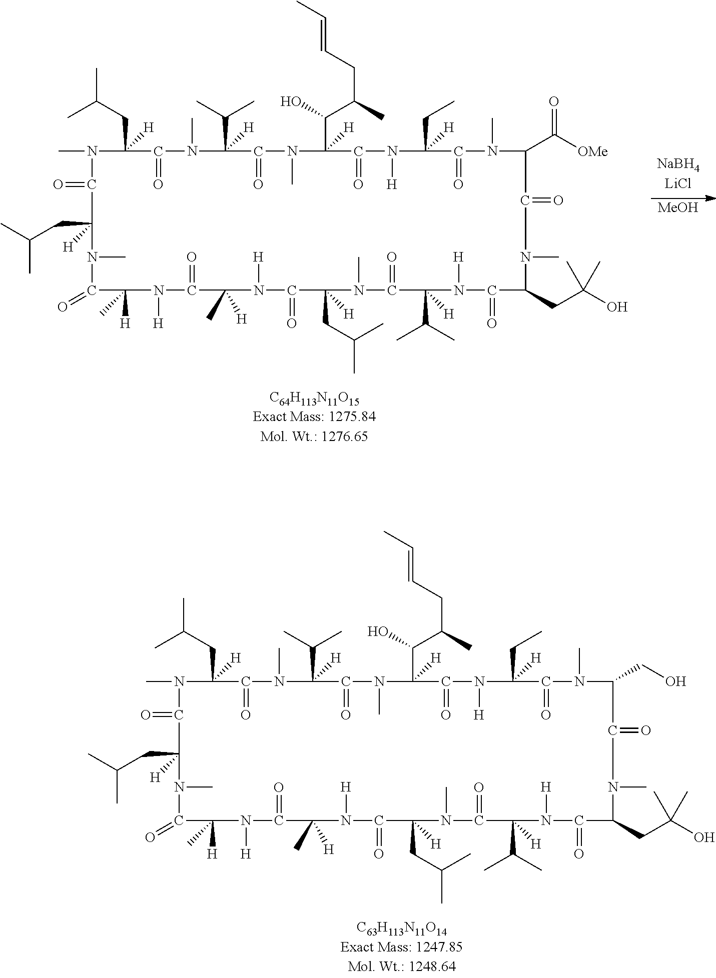 [SMILES: "MeOH" ; C/C=C/C[C@@H](C)[C@@H](O)[C@H]1C(=O)N[C@@H](CC)C(=O)N(C)C(C(=O)OC)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; C/C=C/C[C@@H](C)[C@@H](O)[C@H]1C(=O)N[C@@H](CC)C(=O)N(C)[C@H](CO)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

To a suspension of [α-methoxycarbonyl-Sar]-3-[(γ-hydroxy)-NMeLeu]-4-cyclosporin (3.00 g, 2.35 mmol) and lithium chloride (1.50 g, 35.30 mmol) in methanol (100 ml) was added sodium borohydride (2.50 g, 66.10 mmol) in portions. The mixture was stirred overnight at room temperature. Most of solvent was evaporated under reduced pressure. Dichloromethane (80 ml) and water (50 ml) were added and the mixture was separated. The dichloromethane layer was washed with brine, dried over magnesium sulfate and evaporated under reduced pressure. The residue was purified by chromatography (dichloromethane/methanol=96/4) to give 1.30 g of product of [(R)-α-hydroxymethyl-Sar]-3-[(γ-hydroxy)-NMeLeu]-4-cyclosporin [Molecular Formula: C₆₃H₁₁₃N₁₁O₁₄; Exact Mass: 1247.85; MS (m/z): 1248.48 (M+1)⁺; ¹H NMR spectrum (600 MHz, CDCl₃, δ in ppm): 0.68 (d, J=5.4 Hz, 3H), 0.80-1.00 (m, 30H), 1.07 (d, J=6.0 Hz, 3H), 1.16-1.29 (m, 10H), 1.32 (d, J=7.2 Hz, 3H), 1.39-1.46 (m, 2H), 1.59-1.63 (m, 6H), 1.68-1.83 (m, 7H), 2.02-2.11 (m, 4H), 2.31-2.33 (m, 1H), 2.37-2.42 (m, 2H), 2.67 (s, 6H), 3.09 (s, 3H), 3.19 (s, 3H), 3.20 (s, 3H), 3.22 (s, 3H), 3.47 (s, 3H), 3.72-3.75 (m, 1H), 3.82 (br, 1H), 3.97-3.99 (m, 1H), 4.07-4.10 (m, 1H), 4.50-4.52 (m, 1H), 4.65-4.67 (t, J=8.4 Hz, 1H), 4.79-4.81 (m, 1H), 4.90-4.95 (m, 2H), 5.00-5.05 (m, 2H), 5.09 (d, J=10.8 Hz, 1H), 5.30-5.35 (m, 2H), 5.46 (d, J=6.0 Hz, 1H), 5.52-5.53 (m, 1H), 5.66-5.68 (m, 1H), 7.12 (d, J=7.8 Hz, 1H), 7.47 (d, J=8.4 Hz, 1H), 7.60 (d, J=7.2 Hz, 1H), 7.87-7.89 (d, J=9.6 Hz, 1H)].

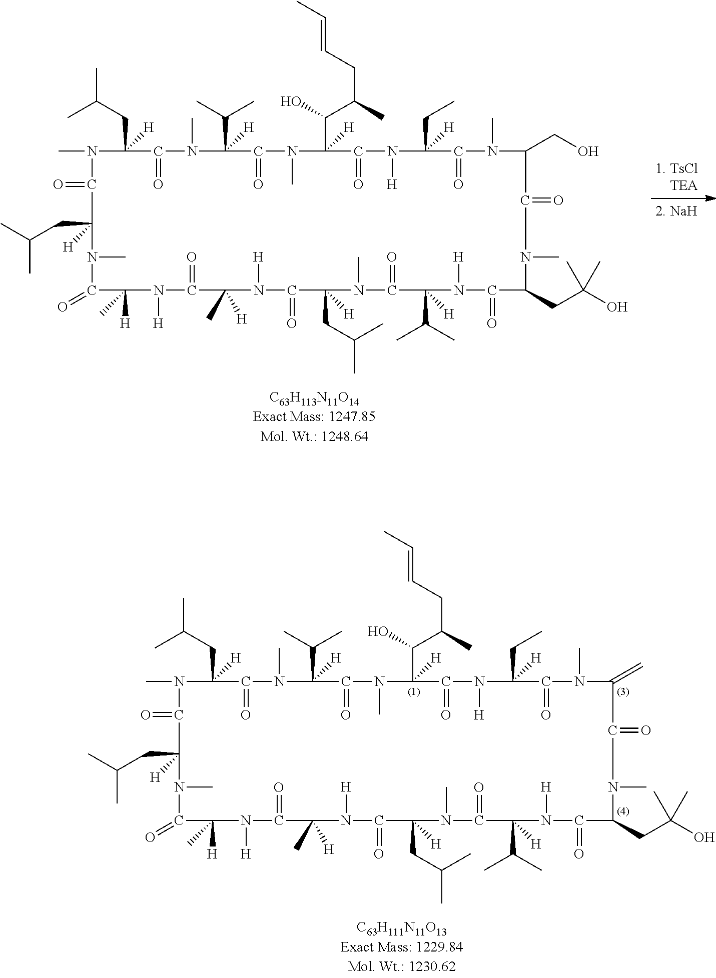 [SMILES: C/C=C/C[C@@H](C)[C@@H](O)[C@H]1C(=O)N[C@@H](CC)C(=O)N(C)C(CO)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; C=C1C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N(C)[C@@H]([C@H](O)[C@H](C)C/C=C/C)C(=O)N[C@@H](CC)C(=O)N1C]

To a solution of [α-hydroxymethyl-Sar]-3-[(γ-hydroxy)-NMeLeu]-4-cyclosporin (250 mg, 0.20 mmol) in dichloromethane (10 mL) at room temperature were added triethylamine (0.33 mL, d 0.726, 2.40 mmol) and triethylamine hydrochloride (95.6 mg, 1.00 mmol), followed by adding p-toluenesulfonyl chloride (0.23 g, 1.20 mmol) under stirring. The mixture was stirred at room temperature overnight. Then the reaction mixture was washed with brine, dried over magnesium sulfate and the solvent was evaporated under reduced pressure. The reaction mixture of [α-chloromethyl-Sar]-3-[(γ-hydroxy)-NMeLeu]-4-cyclosporin [Molecular formula: C₆₃H₁₁₂ClN₁₁O₁₃; Exact Mass: 1265.81; MS (m/z): 1266.32 (M+1)⁺, 1288.43 (M+Na)⁺] and [α-p-toluenesulfonylmethyl-Sar]-3-[(γ-hydroxy)-NMeLeu]-4-cyclosporin [Molecular formula: C₇₀H₁₁₉N₁₁O₁₆S; Exact Mass: 1401.856; MS (m/z): 1402.34 (M+1)⁺, 1424.62 (M+Na)⁺] was directly used in next step reaction without further purification. To a solution of the above mixture in tetrahydrofuran (20 ml) was added sodium hydride (320 mg, 60% in oil, 8 mmol) at 0° C. The mixture was stirred at 0° C. for one hour and then warmed up to room temperature for 30 minutes. The reaction was quenched with a saturated ammonia chloride solution. After removing tetrahydrofuran, the crude product was extracted with ethyl acetate. The ethyl acetate layer was washed with brine, dried over magnesium sulfate and evaporated under reduced pressure. The residue was purified by chromatography on silica gel using ethyl acetate/methanol (20/1) to give 45 mg of product (yield: 18%) [Molecular formula: C₆₃H₁₁₁N₁₁O₁₃; Exact Mass: 1229.84; MS (m/z): 1230.6 (M+1)⁺, 1252.82 (M+Na)⁺; TLC R_{f}: 0.50 (ethyl acetate/methanol=10/1); HPLC RT: 15.38 min. (C8 reverse phase column: 250 mm; acetonitrile/water (0.05% trifluoroacetic acid); operation temperature: 64° C.; detector: 210 nm); ¹H NMR spectrum (600 MHz, CDCl₃, δ in ppm): 0.72 (d, J=5.4 Hz, 3H), 0.84-1.00 (m, 30H), 1.17-1.26 (m, 15H), 1.34 (d, J=6.0 Hz, 3H), 1.44-1.47 (m, 2H), 1.59-1.62 (m, 6H), 1.69-1.76 (m, 4H), 1.94-1.99 (m, 1H), 2.09-2.13 (m, 3H), 2.34-2.37 (m, 3H), 2.65 (s, 3H), 2.67 (s, 3H), 3.09 (s, 3H)), 3.10 (s, 3H), 3.19 (s, 3H), 3.44 (s, 3H), 3.46 (s, 3H), 3.80 (m, 1H), 3.91 (m, 1H), 4.47-4.50 (m, 1H), 4.68-4.71 (t, J=9.0 Hz, 1H), 4.78-4.81 (m, 1H), 4.98-5.02 (m, 2H), 5.06-5.11 (m, 3H), 5.24 (s, 1H), 5.32 (m, 2H), 5.41-5.43 (m, 2H), 5.64-5.66 (m, 1H), 7.11 (d, J=7.2 Hz, 1H), 7.49 (d, J=7.2 Hz, 1H), 7.7 4 (d, J=8.4 Hz, 1H), 7.84 (d, J=9.6 Hz, 1H)].

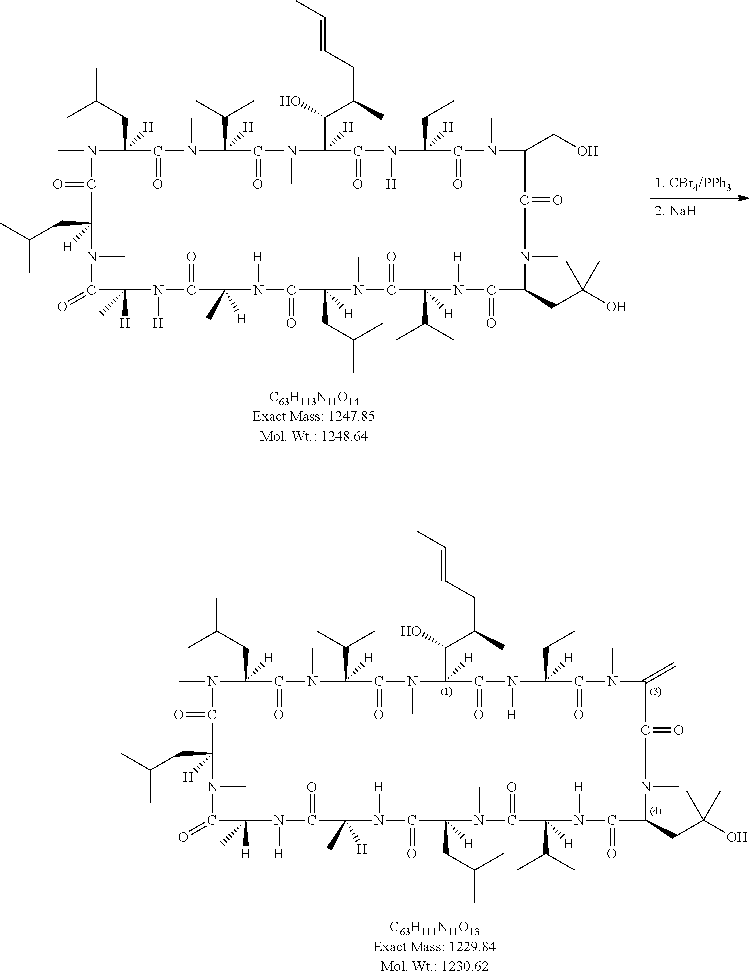 [SMILES: C/C=C/C[C@@H](C)[C@@H](O)[C@H]1C(=O)N[C@@H](CC)C(=O)N(C)C(CO)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; C=C1C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N(C)[C@@H]([C@H](O)[C@H](C)C/C=C/C)C(=O)N[C@@H](CC)C(=O)N1C]

[(R)-α-Hydroxymethyl-Sar]-3-[(γ-hydroxy)-NMeLeu]-4-cyclosporin (crude, 2.00 g), carbon tetrabromide (2.66 g, 8.02 mmol) and triphenylphosphine (2.11 g, 8.02 mmol) were dissolved in dichloromethane (30 ml). The mixture was stirred under nitrogen at room temperature for two hours. Then the mixture was added into a suspension of sodium hydride (60% dispersion in mineral oil) (0.77 g, 19.25 mmol) in tetrahydrofuran (30 ml) under nitrogen at 0° C. The mixture was stirred at 0° C. for one hour. Most of solvents then were evaporated under reduced pressure. The residue was treated with water (10 ml) slowly at 0° C. Ethyl acetate (30 ml) and water (30 ml) were added and the mixture was separated. The ethyl acetate layer was washed with brine, dried over magnesium sulfate and evaporated under reduced pressure. The residue was purified by chromatography (hexane/acetone from 90/10 to 70/30) to give 0.68 g product of [α-methylene-Sar]-3-[(γ-hydroxy)-NMeLeu]-4-cyclosporin [Molecular Formula: C₆₃H₁₁₁N₁₁O₁₃; Exact Mass: 1229.84; MS (m/z): 1230.50 (M+1)⁺, 1252.68 (M+Na)⁺; TLC R_{f}: 0.50 (ethyl acetate/methanol=10/1); HPLC RT: 15.36 min. (C8 reverse phase column: 250 mm; acetonitrile/water (0.05% trifluoroacetic acid); operation temperature: 64° C.; detector: 210 nm)].

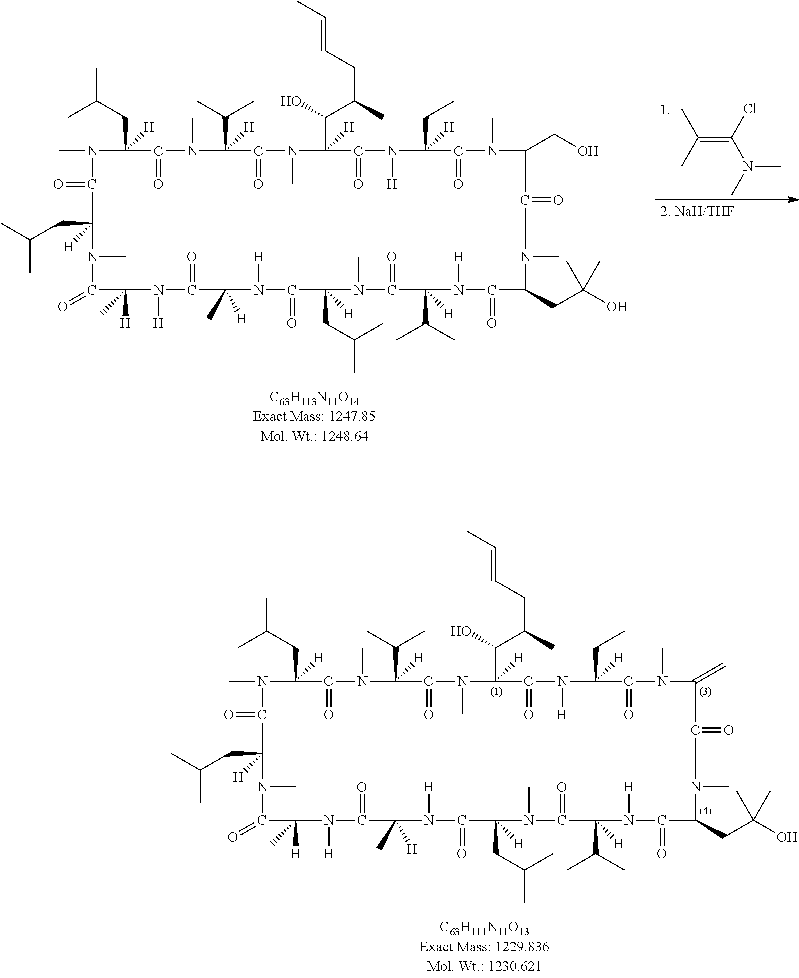

To a solution of [(R)-α-hydroxymethyl-Sar]-3-[(γ-hydroxy)-NMeLeu]-4-cyclosporin (250 mg, 0.20 mmol) in methylene chloride (10 mL) was added dropwise 1-chloro-N,N,2-trimethyl-1-propenylamine (131 μl, d 1.01, 1.0 mmol) at 0° C. under nitrogen atmosphere. After stirred for 30 minutes at 0° C., the mixture was allowed to warm to room temperature and stirred for another hour. The reaction mixture was washed with sodium bicarbonate solution, brine, dried over magnesium sulfate and evaporated under reduced pressure. The crude product containing [α-chloromethyl-Sar]-3-[(γ-hydroxy)-NMeLeu]-4-cyclosporin [Molecular formula: C₆₃H₁₁₂ClN₁₁O₁₃; Exact Mass: 1265.81; MS (m/z): 1266.32 (M+1)⁺, 1288.43 (M+Na)⁺] was used in next step reaction without further purification. To a solution of the above crude product in tetrahydrofuran (20 ml) was added sodium hydride (320 mg, 60% in oil, 8 mmol) at 0° C. under stirring. The mixture was stirred at 0° C. for one hour and then warmed up to room temperature for another 30 minutes. The reaction was then quenched with a saturated ammonia chloride solution. After removing tetrahydrofuran, the residue was extracted with ethyl acetate. The ethyl acetate layer was washed with brine, dried over magnesium sulfate and evaporated under reduced pressure. The residue was purified by chromatography on silica gel using ethyl acetate/methanol (20/1) to give 33 mg of product (yield: 13%) [Molecular formula: C₆₃H₁₁₁N₁₁O₁₃; Exact Mass: 1229.84; MS (m/z): 1230.45 (M+1)⁺, 1252.65 (M+Na)⁺; TLC R_{f}: 0.50 (ethyl acetate/methanol=10/1); HPLC RT: 15.36 min. (C8 reverse phase column: 250 mm; acetonitrile/water (0.05% trifluoroacetic acid); operation temperature: 64° C.; detector: 210 nm)].

### Example 3

### [α-Methylene-Sar]-3-[(γ-methoxy)-NMeLeu]-4-cyclosporin

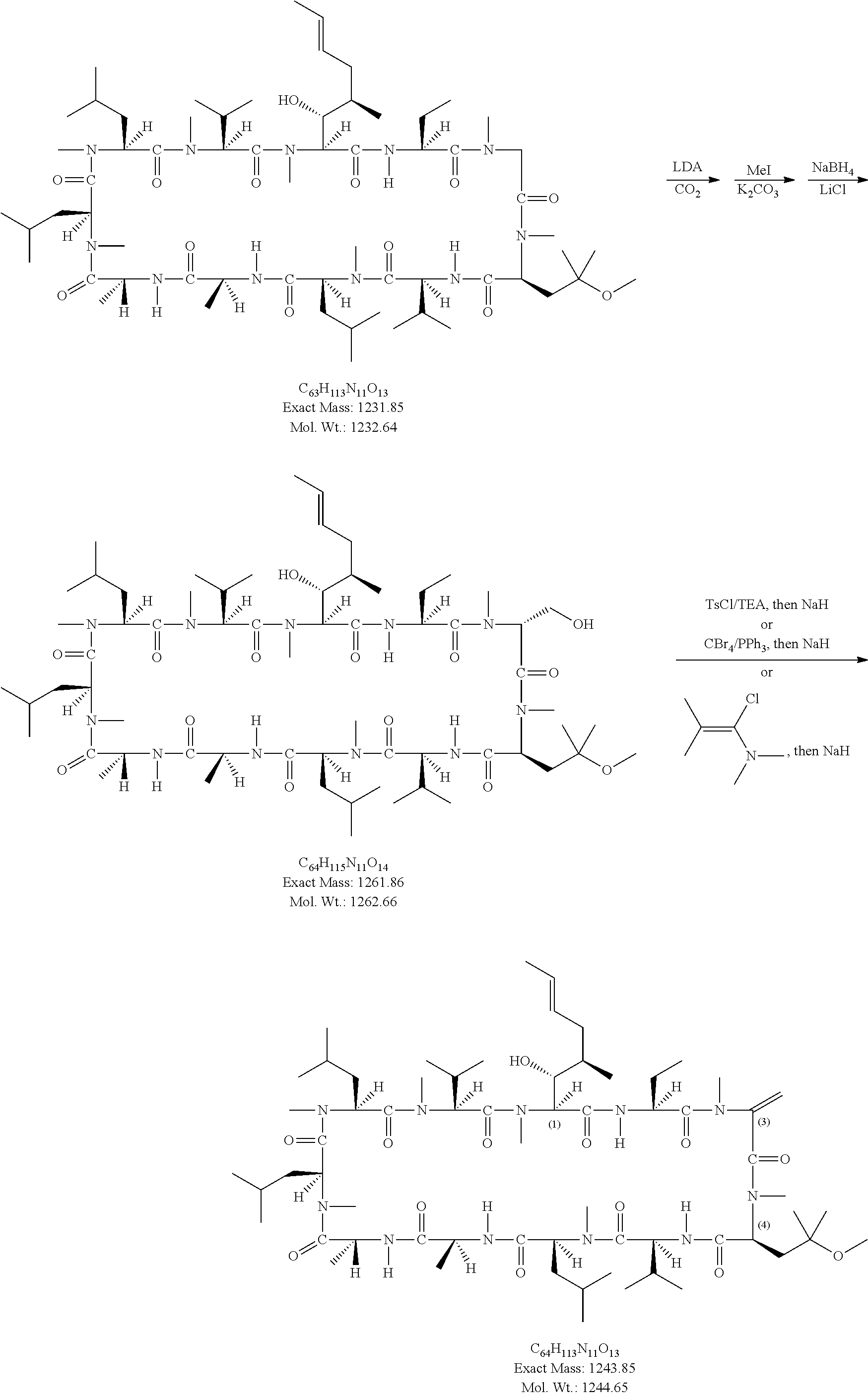

[α-Methylene-Sar]-3-[(γ-methoxy)-NMeLeu]-4-cyclosporin is prepared according to a method analogous to that described in Example 2.

### Example 4

### [α-Methylene-Sar]-3-[(γ-allyloxy)-NMeLeu]-4-cyclosporin

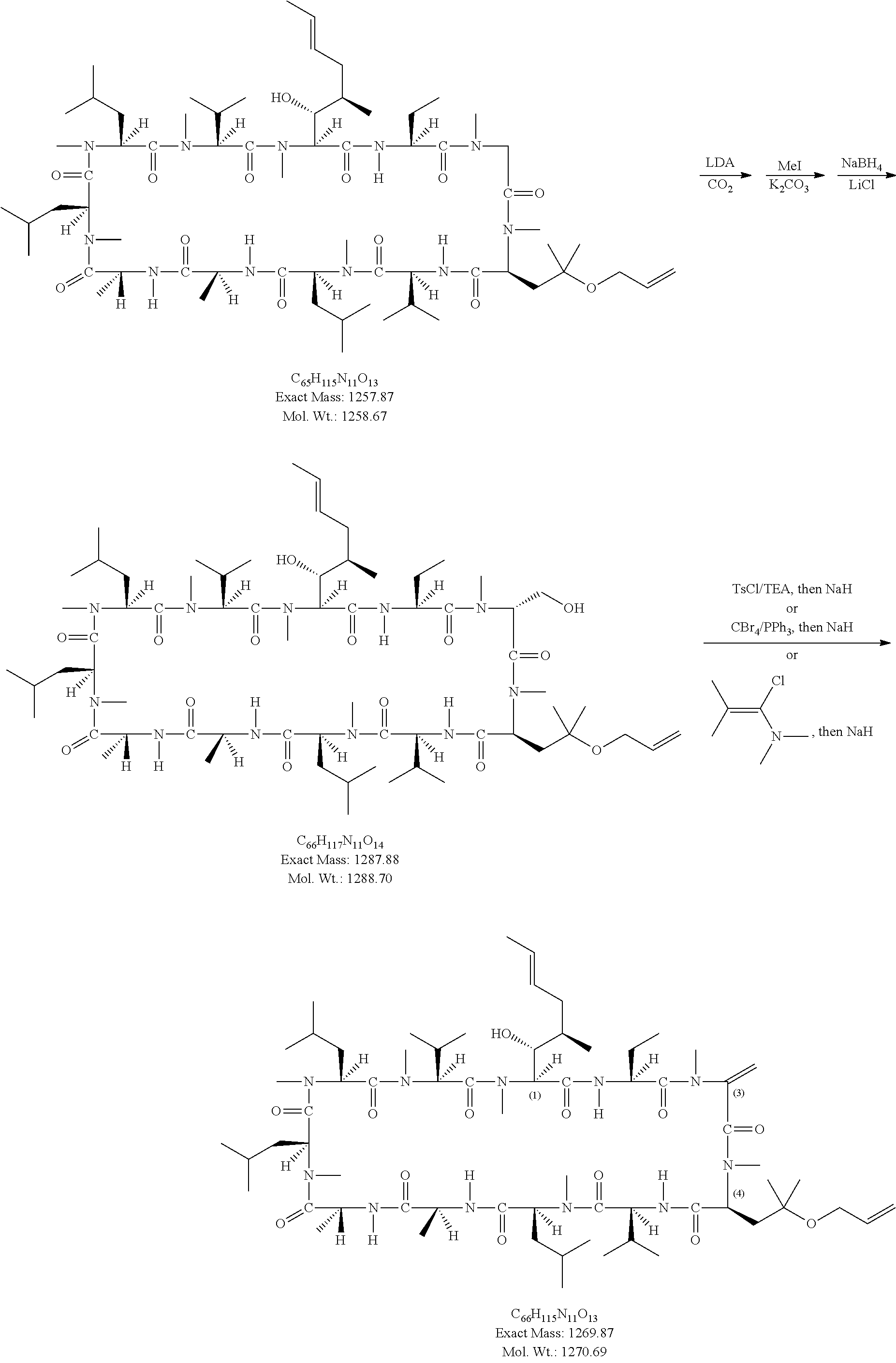

[α-Methylene-Sar]-3-[(γ-allyloxy)-NMeLeu]-4-cyclosporin is prepared according a method analogous to that described in Example 2.

### Example 5

### [α-Methylene-Sar]-3-[NMeVal]-4-cyclosporin

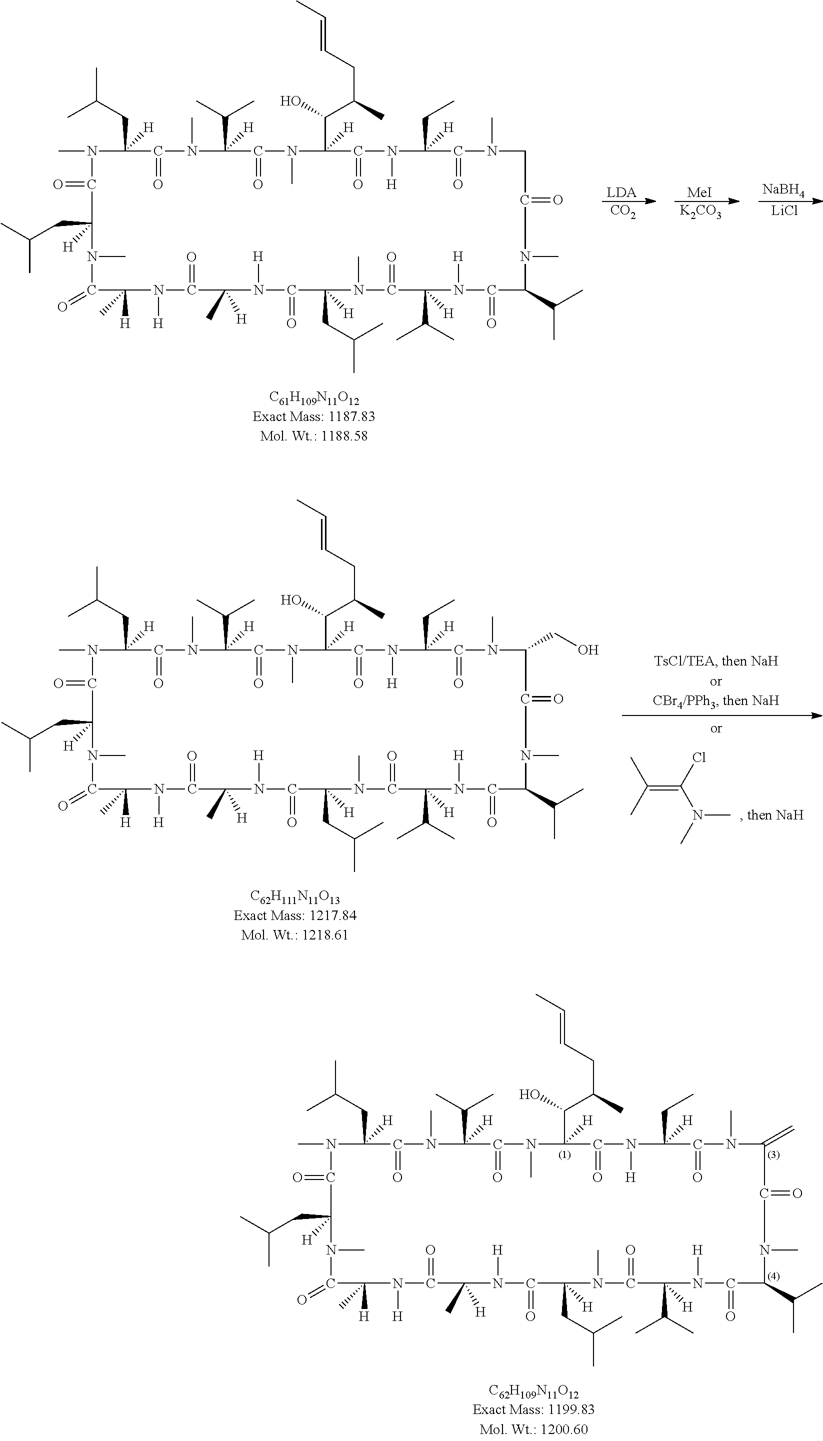

[α-Methylene-Sar]-3-[NMeVal]-4-cyclosporin is prepared according to a method analogous to that described in Example 2.

### Example 6

### [α-Methylene-Sar]-3-[NMeIle]-4-cyclosporin

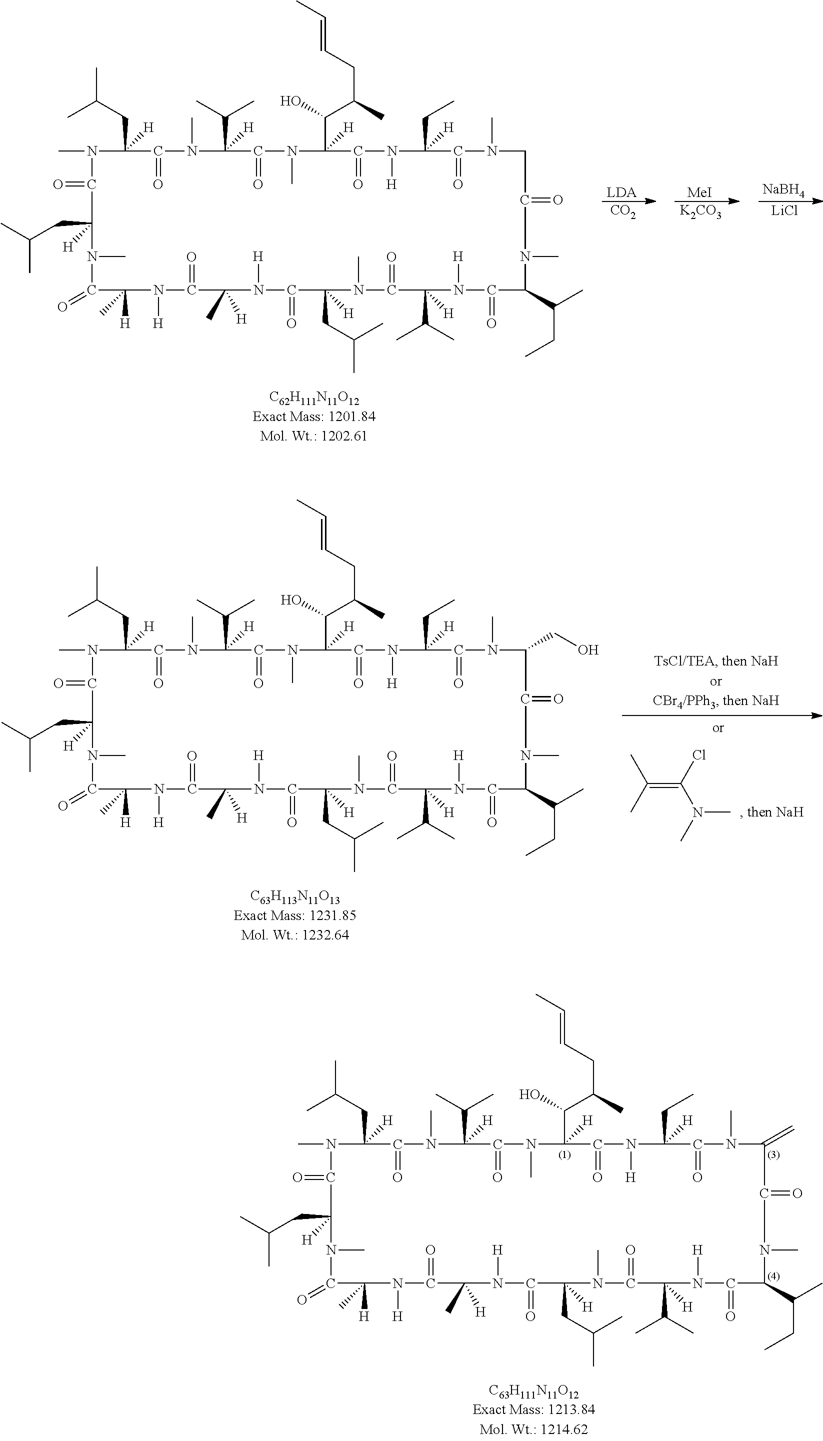

[α-Methylene-Sar]-3-[NMeIle]-4-cyclosporin is prepared according to a method analogous to that described in Example 2.

### Example 7

### [(R)-2-Nitroethyl-Sar]-3-cyclosporin

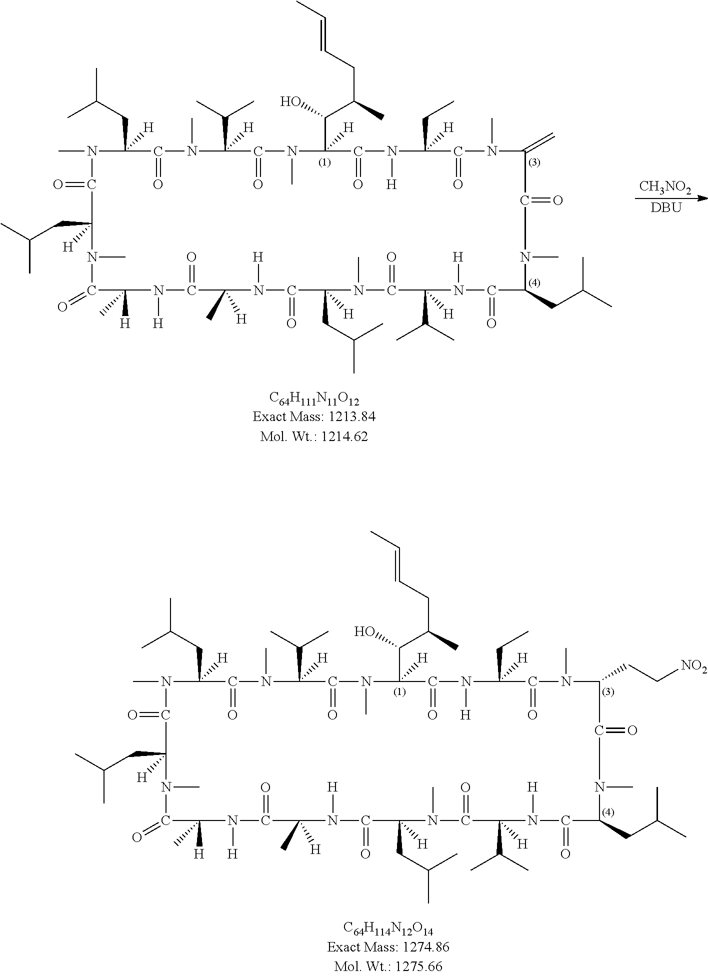

To a solution of [α-methylene-Sar]-3-cyclosporin (1.00 g, 0.82 mmol) in nitromethane (15 ml) was added 1,8-diazabicyclo[5.4.0]undec-7-ene (FW 152.24, 1.00 g, 6.60 mmol). After stirred at room temperature for two days, the reaction mixture was concentrated and diluted with water. The mixture was extracted with dichloromethane. The dichloromethane layer was washed with aqueous citric acid solution and brine, dried over magnesium sulfate, filtered and concentrated. The residue was purified by chromatography (hexane/acetone=3/1) to give the product [Molecular formula: C₆₄H₁₁₄N₁₂O₁₄; Exact Mass: 1274.86; MS (m/z): 1275.54 (M+1)⁺].

### Example 8

### [(R)-2-Nitroethyl-Sar]-3-[(γ-hydroxy)-NMeLeu]-4-cyclosporin and [(S)-2-nitroethyl-Sar]-3-[(γ-hydroxy)-NMeLeu]-4-cyclosporin

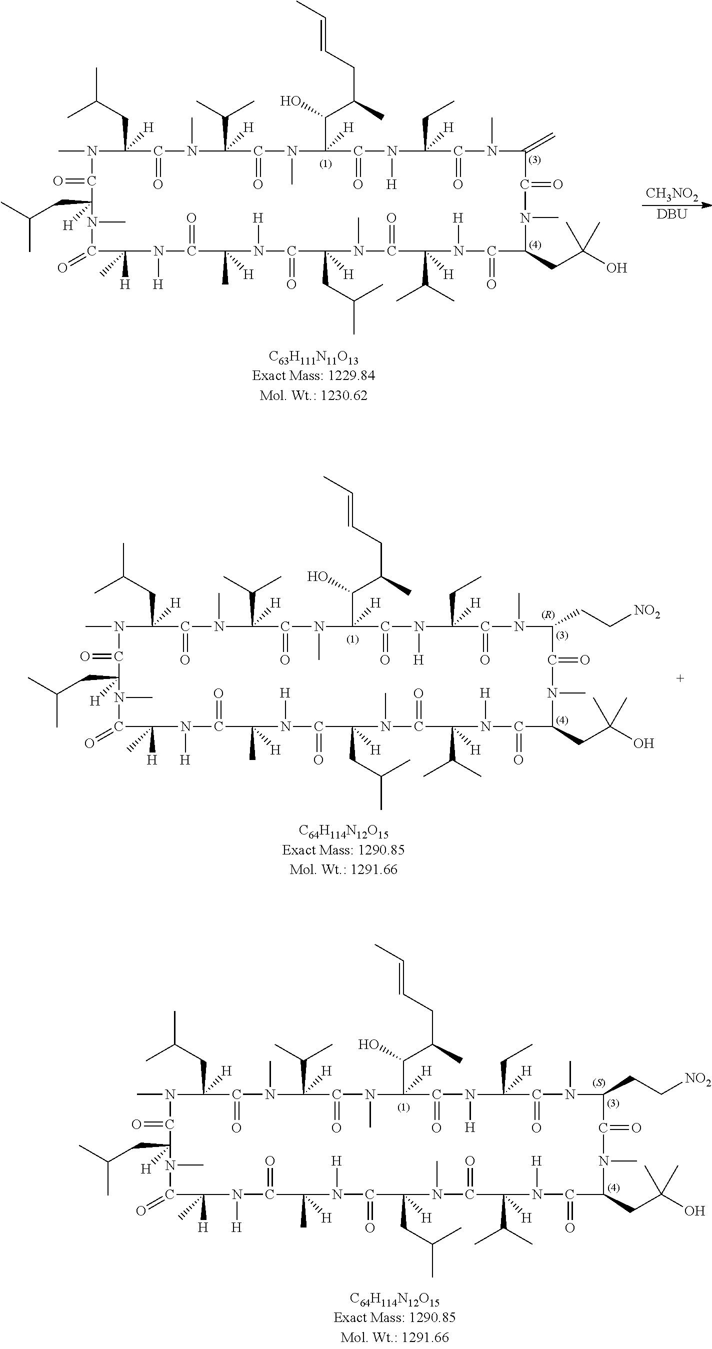

To a solution of [α-methylene-Sar]-3-[(γ-hydroxy)-NMeLeu]-4-cyclosporin (1.60 g, 1.30 mmol) in nitromethane (20 ml) was added 1,8-diazabicyclo[5.4.0]undec-7-ene (4 ml), and then the reaction mixture was stirred at room temperature overnight. The mixture was concentrated, diluted with water, and extracted with dichloromethane. The organic layer was washed with aqueous citric acid solution and brine, dried over magnesium sulfate, filtered and concentrated. The residue was purified by chromatography (dichloromethane/methanol=97/3) to give the product (R-isomer) 600 mg [Molecular formula: C₆₄H₁₁₄N₁₂O₁₅; Exact Mass: 1290.85; MS (m/z): 1291.72 (M+1)⁺. HPLC RT: 14.95 minutes], and (S-isomer) 360 mg [Molecular formula: C₆₄H₁₁₄N₁₂O₁₅; Exact Mass: 1290.85; MS (m/z): 1291.72 (M+1)⁺; HPLC RT: 14.43 min. (C8 reverse phase column: 250 mm; acetonitrile/water (0.05% trifluoroacetic acid); operation temperature: 64° C.; detector: 210 nm)].

### Example 9

### [(R)-2-Nitroethyl-Sar]-3-[(γ-methoxy)-NMeLeu]-4-cyclosporin

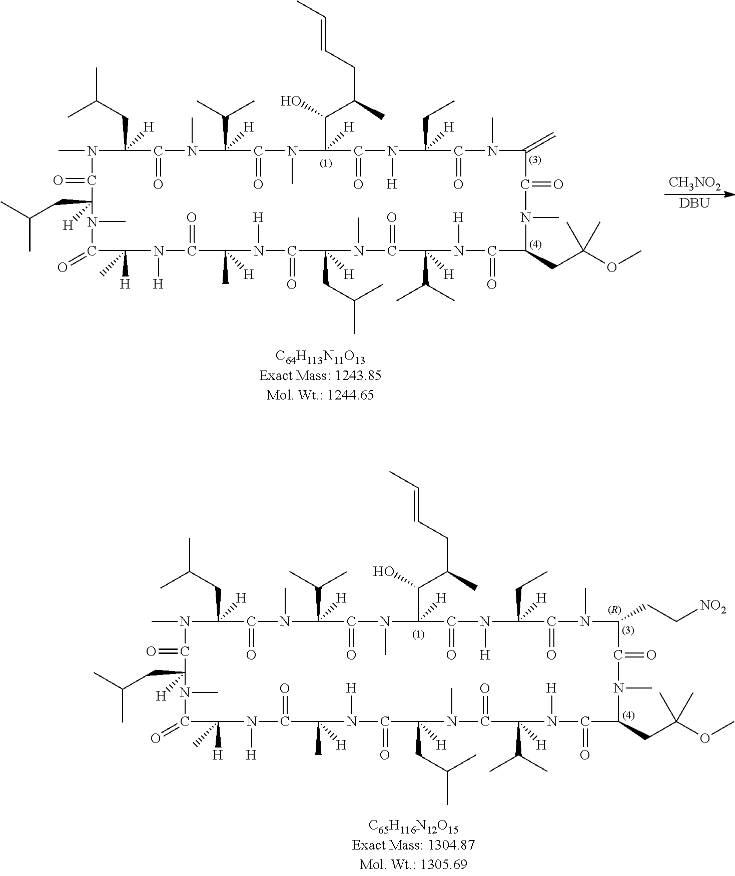

[(R)-2-Nitroethyl-Sar]-3-[(γ-methoxy)-NMeLeu]-4-cyclosporin is prepared according to a method analogous to that described in Example 8.

### Example 10

### [(R)-2-Nitroethyl-Sar]-3-[(γ-allyloxy)-NMeLeu]-4-cyclosporin

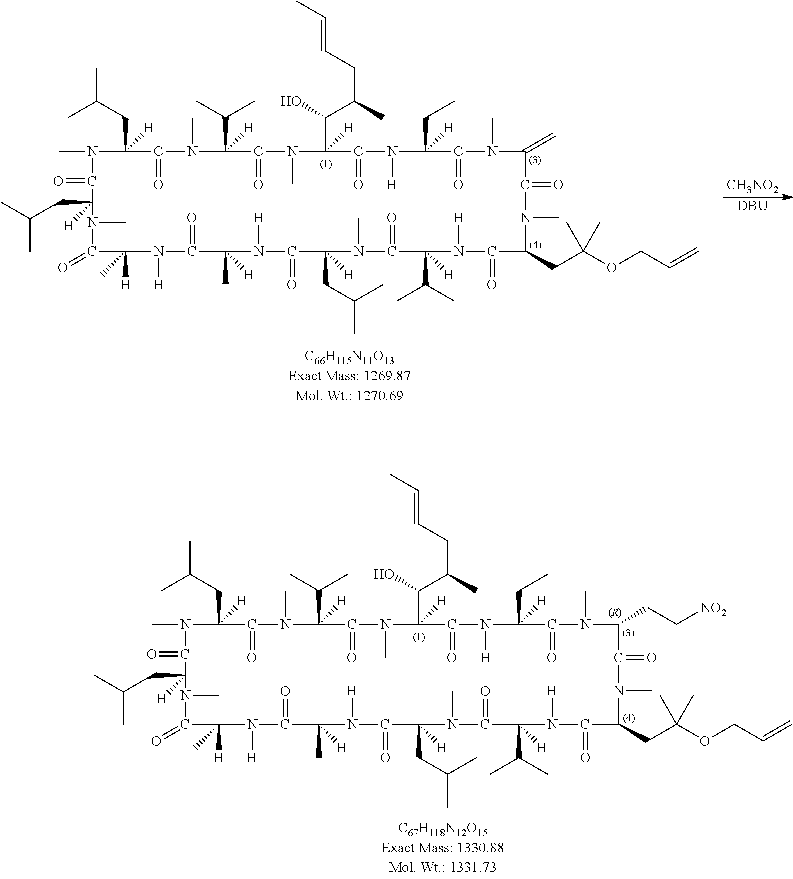

[(R)-2-Nitroethyl-Sar]-3-[(γ-allyloxy)-NMeLeu]-4-cyclosporin is prepared according to a method analogous to that described in Example 8.

### Example 11

### [(R)-2-Nitroethyl-Sar]-3-[NMeVal]-4-cyclosporin

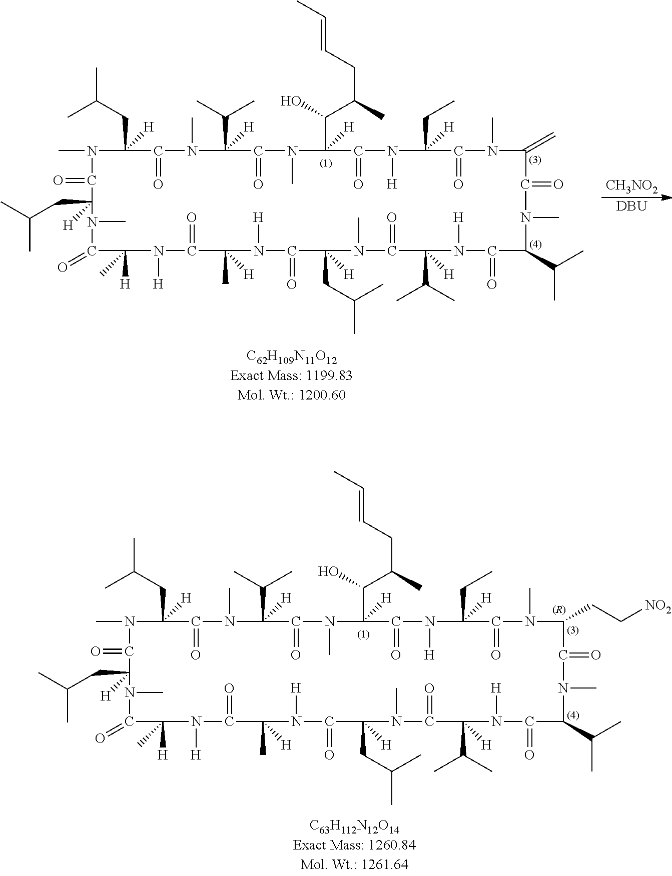

[(R)-2-Nitroethyl-Sar]-3-[NMeVal]-4-cyclosporin is prepared according to a method analogous to that described in Example 8.

### Example 12

### [(R)-2-Nitroethyl-Sar]-3-[NMeIle]-4-cyclosporin

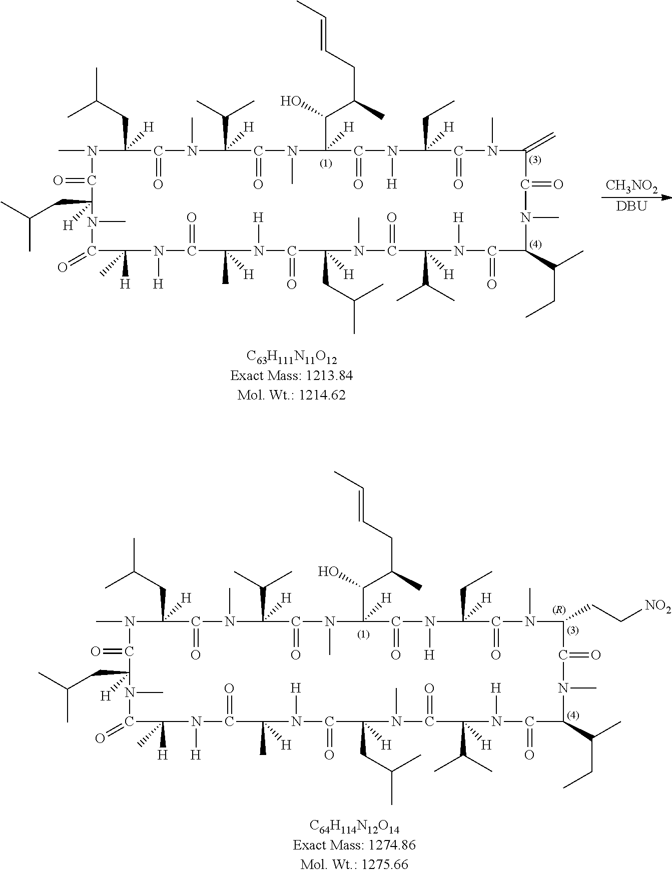

[(R)-2-Nitroethyl-Sar]-3-[NMeIle]-4-cyclosporin is prepared according to a method analogous to that described in Example 8.

### Example 13

### [(R)-2-Aminoethyl-Sar]-3-cyclosporin

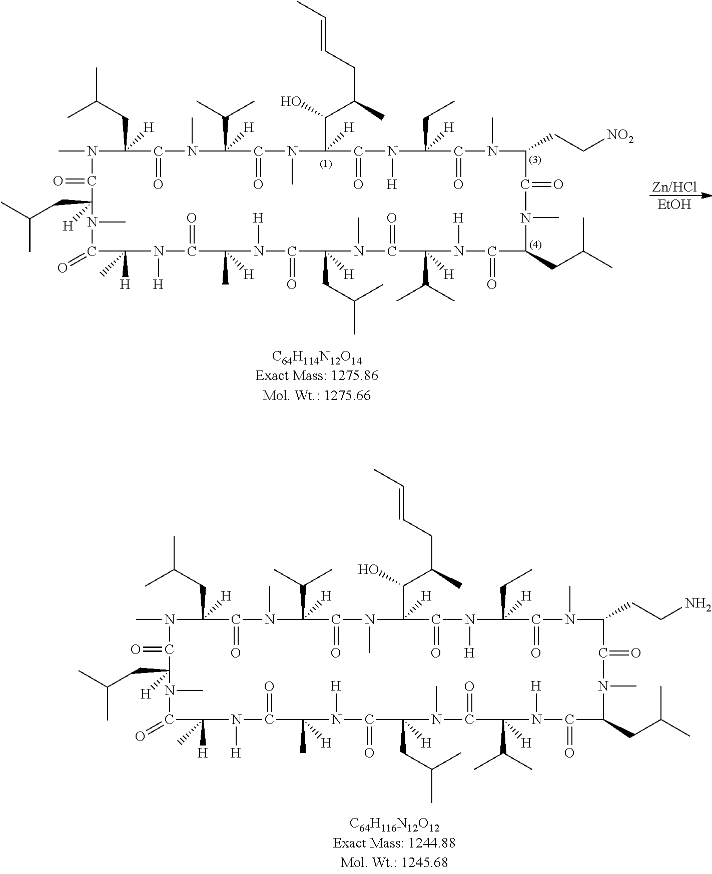

To a mixture of [(R)-2-nitroethyl-Sar]-3-cyclosporin (210 mg, 0.16 mmol) and zinc (FW 65.38, 1.00 g, 15.3 mmol) in ethanol (20 ml) was added 10% aqueous hydrochloric acid (10 ml). The reaction mixture was stirred at room temperature overnight (monitored by LC-MS) and filtered. The filter cake was washed with ethanol. The filtrate was concentrated and diluted with dichloromethane. The organic layer was washed with aqueous sodium bicarbonate solution and brine, dried over magnesium sulfate, filtered and concentrated. The residue was purified by chromatography (dichloromethane/methanol=95/5) to give the product [Molecular formula: C₆₄H₁₁₆N₁₂O₁₂; Exact Mass: 1244.88; MS (m/z): 1245.54 (M+1)⁺].

### Example 14

### [(R)-2-Aminoethyl-Sar]-3-[(γ-hydroxy)-NMeLeu]-4-cyclosporin

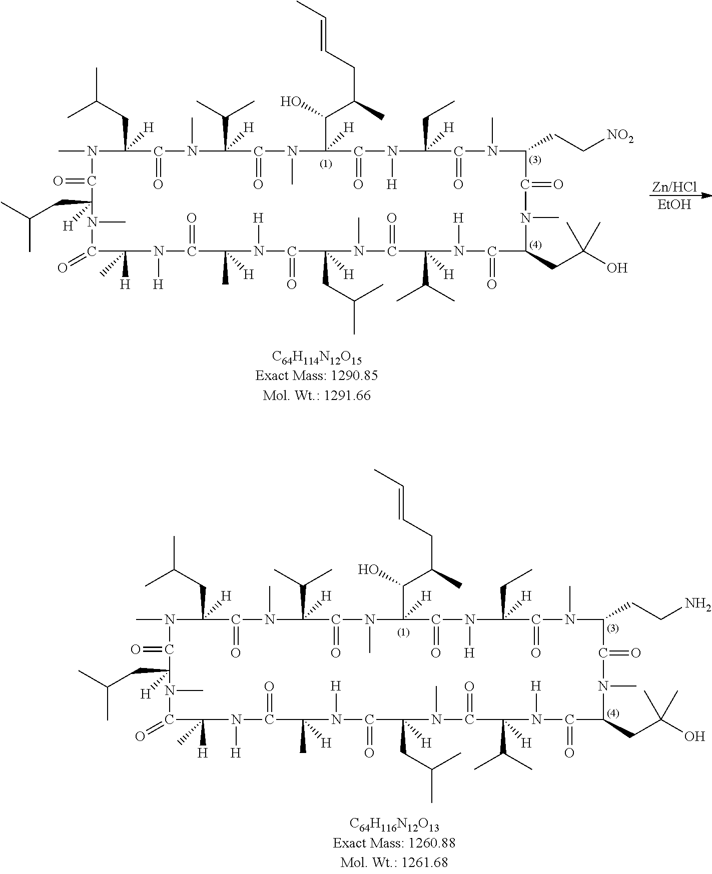

To a mixture of [(R)-2-nitroethyl-Sar]-3-[(γ-hydroxy)-NMeLeu]-4-cyclosporin (400 mg, 0.31 mmol) and zinc (1.50 g) in ethanol (30 ml) was added 10% aqueous hydrochloric acid (30 ml). The reaction mixture was stirred at room temperature overnight (monitored by LC-MS). The mixture was filtered. The filter cake was washed with ethanol. The filtrate was concentrated and diluted with dichloromethane. The organic layer was washed with aqueous sodium bicarbonate solution and brine, dried over magnesium sulfate, filtered and concentrated. The residue was purified by chromatography (dichloromethane/methanol=95/5) to give the product [Molecular formula: C₆₄H₁₁₆N₁₂O₁₃; Exact Mass: 1260.88; MS (m/z): 1261.70 (M+1)⁺].

### Example 15

### [(R)-2-Aminoethyl-Sar]-3-[(γ-methoxy)-NMeLeu]-4-cyclosporin

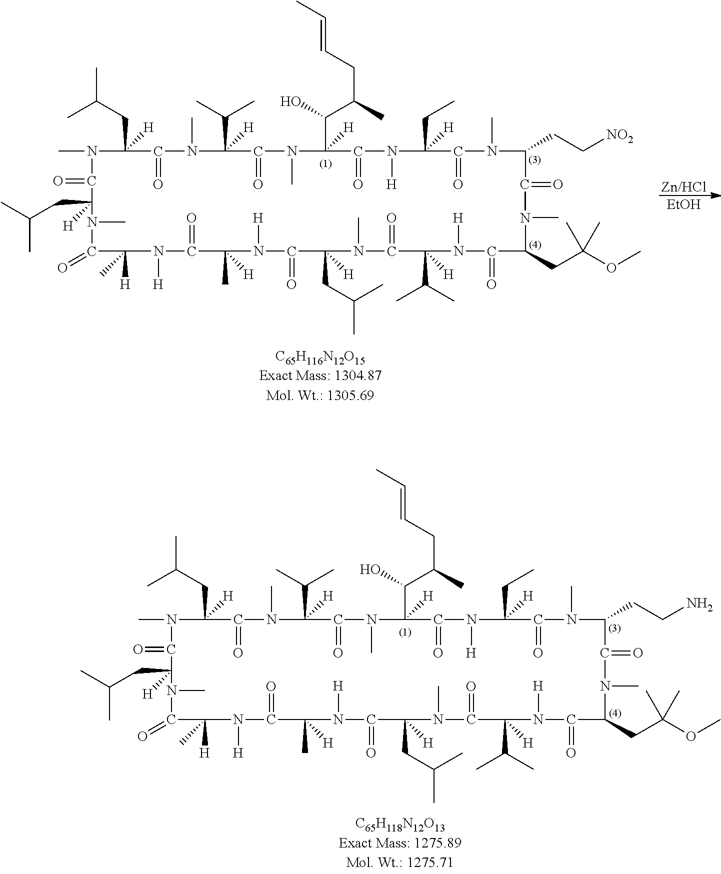

[(R)-2-Aminoethyl-Sar]-3-[(γ-methoxy)-NMeLeu]-4-cyclosporin is prepared according to a method analogous to that described in Example 14.

### Example 16

### [(R)-2-Aminoethyl-Sar]-3-[(γ-allyloxy)-NMeLeu]-4-cyclosporin

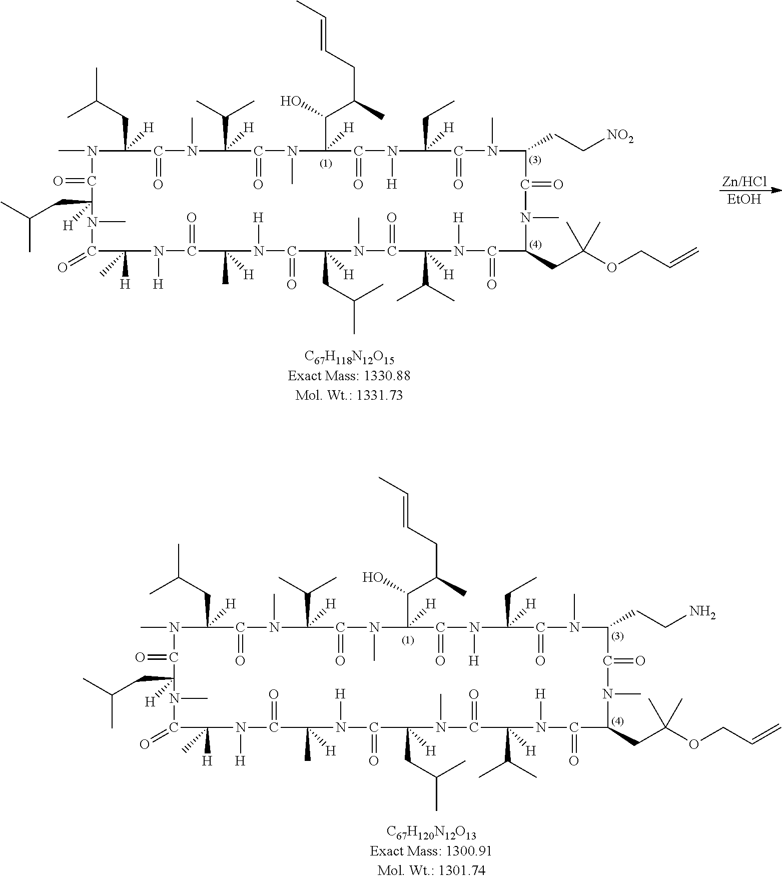

[(R)-2-Aminoethyl-Sar]-3-[(γ-allyloxy)-NMeLeu]-4-cyclosporin is prepared according to a method analogous to that described in Example 14.

### Example 17

### [(R)-2-Aminoethyl-Sar]-3-[NMeVal]-4-cyclosporin

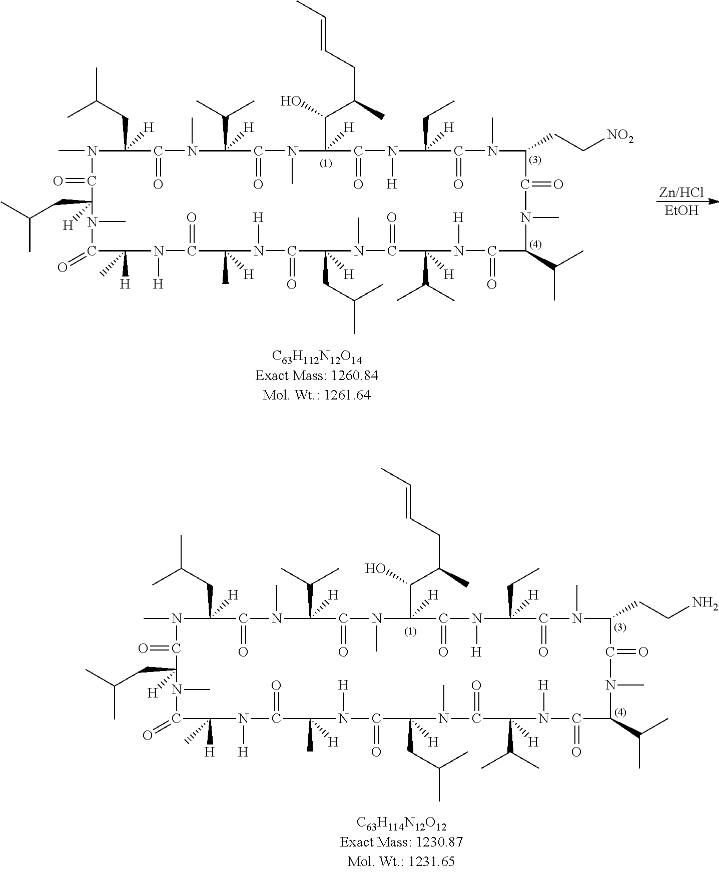

[(R)-2-Aminoethyl-Sar]-3-[NMeVal]-4-cyclosporin is prepared according to a method analogous to that described in Example 14.

### Example 18

### [(R)-2-Aminoethyl-Sar]-3-[NMeIle]-4-cyclosporin

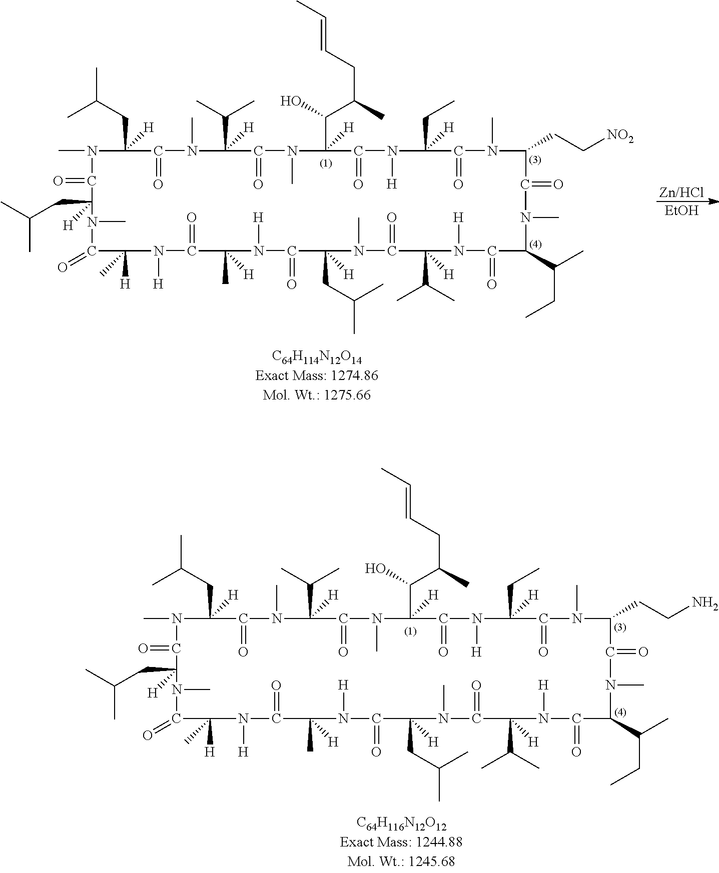

[(R)-2-Aminoethyl-Sar]-3-[NMeIle]-4-cyclosporin is prepared according to a method analogous to that described in Example 14.

### Example 19

### [(R)-2-(N,N-Dimethylamino)ethyl-Sar]-3-cyclosporin

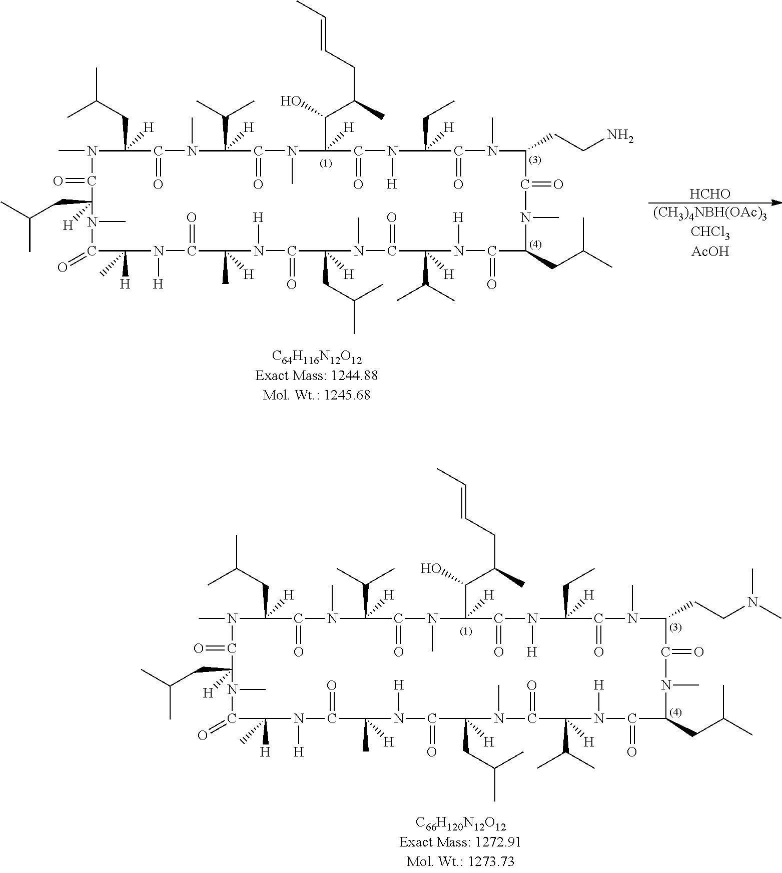

To a solution of [(R)-2-aminoethyl-Sar]-3-cyclosporine (122 mg, 0.1 mmol) in chloroform (6 ml) were added formaldehyde aqueous 37% solution (0.6 ml) and acetic acid (6 drops). The reaction mixture was stirred at room temperature for 5 minutes. Then tetramethylammonium triacetoxyborohydride (FW 263.10, 131 mg, 0.50 mmol) was added and the reaction mixture was continued to stir for another hour. The mixture was diluted with dichloromethane. The organic layer was washed with aqueous sodium bicarbonate solution and brine, dried over magnesium sulfate, filtered and concentrated. The residue was purified by chromatography (dichloromethane/methanol=96/4) to give the product [Molecular formula: C₆₆H₁₂₀N₁₂O₁₂; Exact Mass: 1272.91; MS (m/z): 1273.70 (M+1)⁺; TLC R_{f}: 0.27 (dichloromethane/methanol=95/5); HPLC RT: 12.42 min. (C8 reverse phase column: 150 mm; acetonitrile/water (0.05% trifluoroacetic acid); operation temperature: 64° C.; detector: 210 nm)].

### Example 20

### [(R)-2-(N,N-Dimethylamino)ethyl-Sar]-3-[(γ-hydroxy)-NMeLeu]-4-cyclosporin

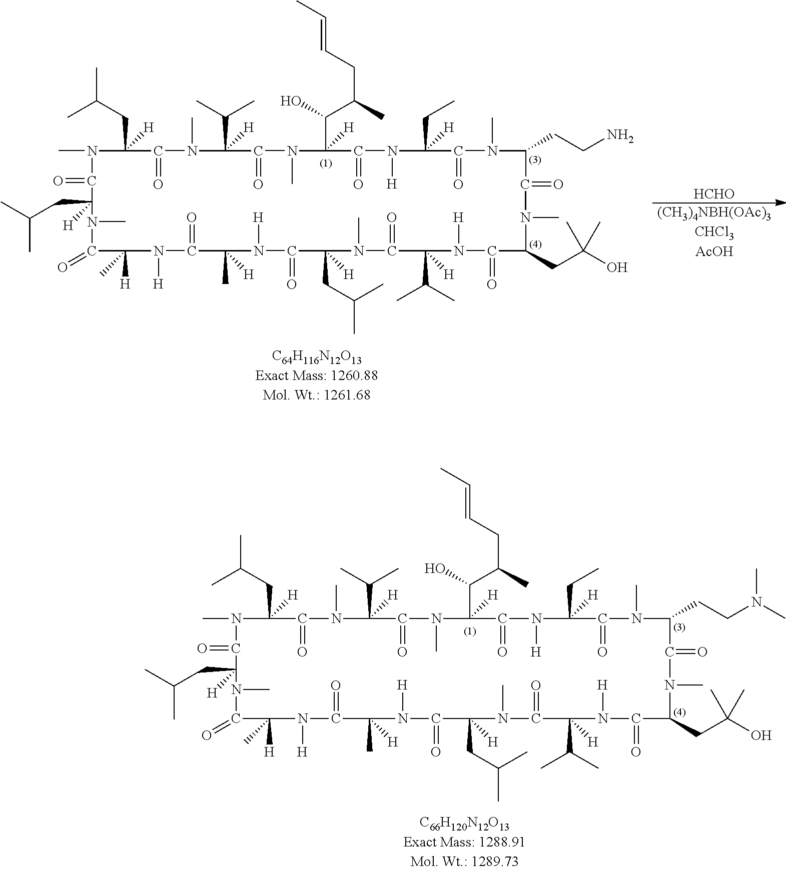

To a solution of [(R)-2-aminoethyl-Sar]-3-[(γ-hydroxy)-NMeLeu]-4-cyclosporin (180 mg, 0.14 mmol) in chloroform (6 ml) were added formaldehyde aqueous 37% solution (0.8 ml) and acetic acid (8 drops). The reaction mixture was stirred at room temperature for 5 minutes. Then tetramethylammonium triacetoxyborohydride (FW 263.10, 200 mg, 0.76 mmol) was added and the reaction mixture was continued to stir for 1 hour. The mixture was diluted with dichloromethane. The organic layer was washed with aqueous sodium bicarbonate solution and brine, dried over magnesium sulfate, filtered and concentrated. The residue was purified by chromatography (dichloromethane/methanol=95/5) to give the product [Molecular formula: C₆₆H₁₂₀N₁₂O₁₃; Exact Mass: 1288.91; MS (m/z): 1289.76 (M+1)⁺; TLC R_{f}: 0.32 (dichloromethane/methanol=9/1); HPLC RT: 11.14 min. (C8 reverse phase column: 150 mm; acetonitrile/water (0.05% trifluoroacetic acid); operation temperature: 64° C.; detector: 210 nm)].

### Example 21

### [(R)-2-(N,N-Dimethylamino)ethyl-Sar]-3-[(γ-methoxy)-NMeLeu]-4-cyclosporin

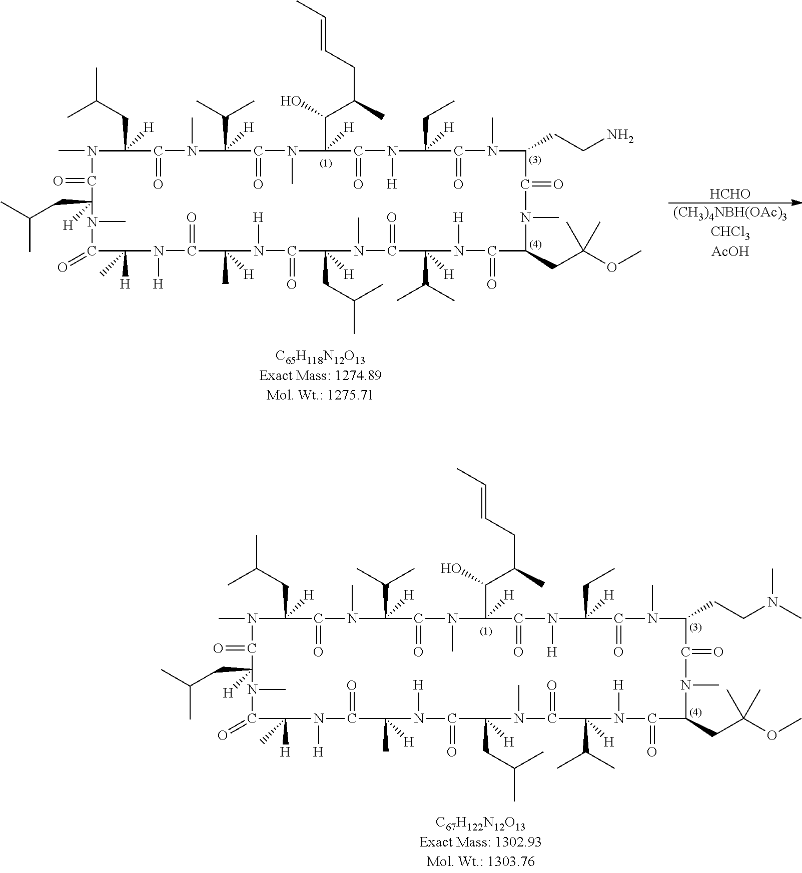

[(R)-2-(N,N-Dimethylamino)ethyl-Sar]-3-[(γ-methoxy)-NMeLeu]-4-cyclosporin is prepared according to a method analogous to that described in Example 20.

### Example 22

### [(R)-2-(N,N-Dimethylamino)ethyl-Sar]-3-[(γ-allyloxy)-NMeLeu]-4-cyclosporin

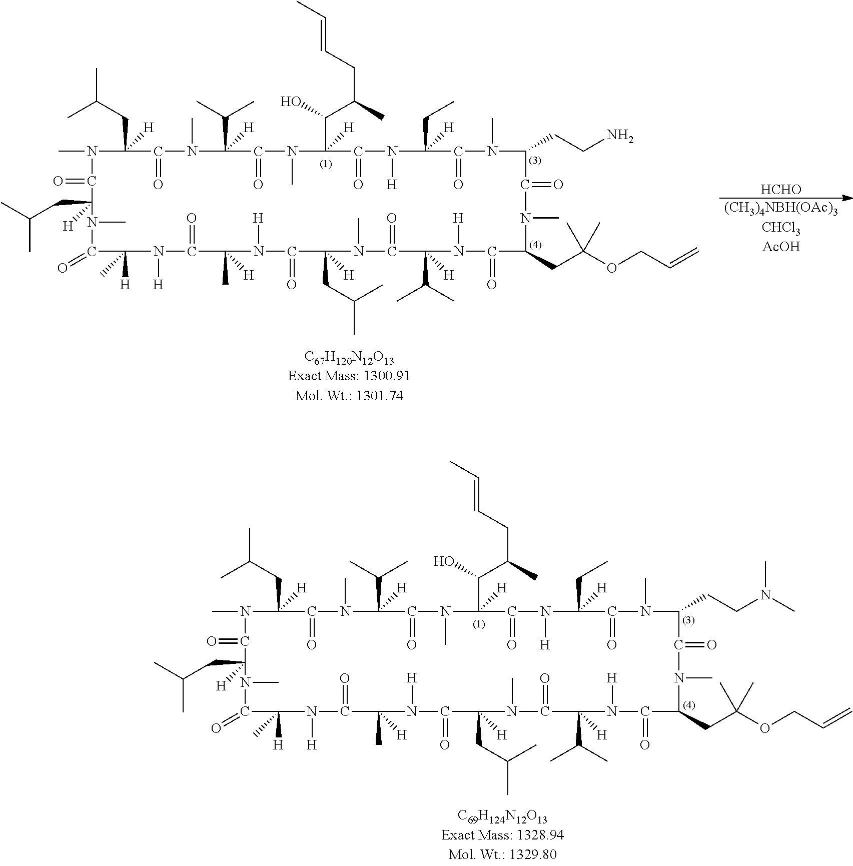

[(R)-2-(N,N-Dimethylamino)ethyl-Sar]-3-[(γ-allyloxy)-NMeLeu]-4-cyclosporin is prepared according to a method analogous to that described in Example 20.

### Example 23

### [(R)-2-(N,N-Dimethylamino)ethyl-Sar]-3-[NMeVal]-4-cyclosporin

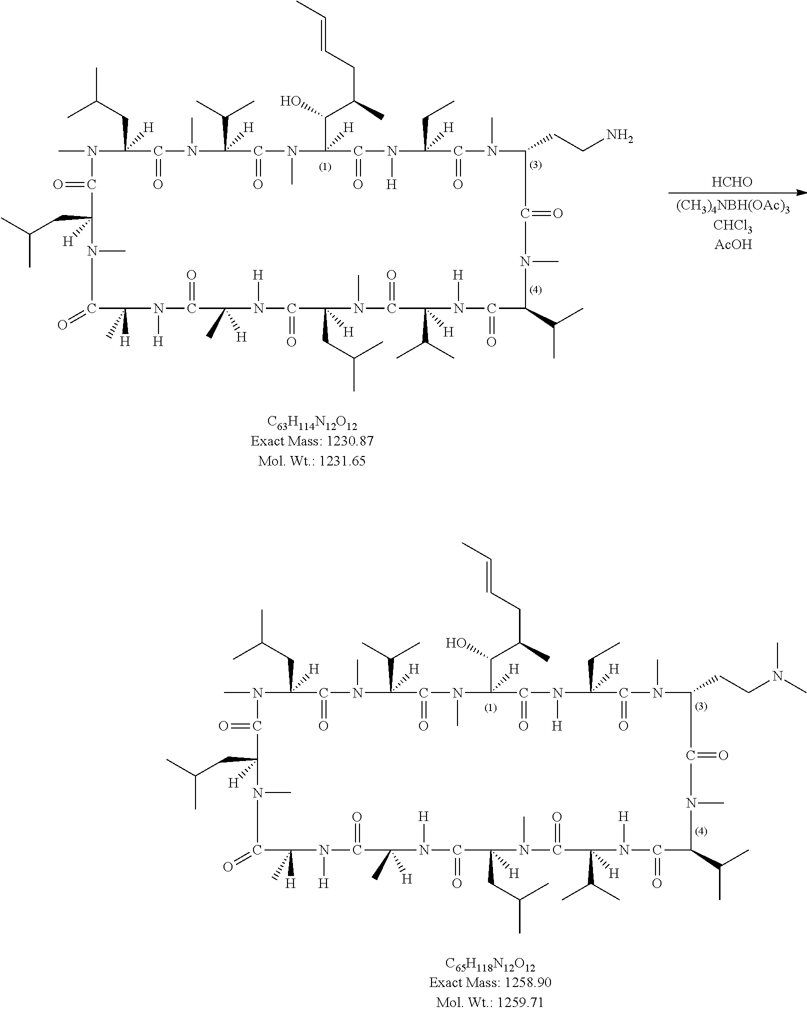 [SMILES: C/C=C/C[C@@H](C)[C@@H](O)[C@H]1C(=O)N[C@@H](CC)C(=O)N(C)[C@H](CCN)C(=O)N(C)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; "HCHO" ; C/C=C/C[C@@H](C)[C@@H](O)[C@H]1C(=O)N[C@@H](CC)C(=O)N(C)[C@H](CCN(C)C)C(=O)N(C)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

[(R)-2-(N,N-Dimethylamino)ethyl-Sar]-3-[NMeVal]-4-cyclosporin is prepared according to a method analogous to that described in Example 20.

### Example 24

### [(R)-2-(N,N-Dimethylamino)ethyl-Sar]-3-[NMeIle]-4-cyclosporin

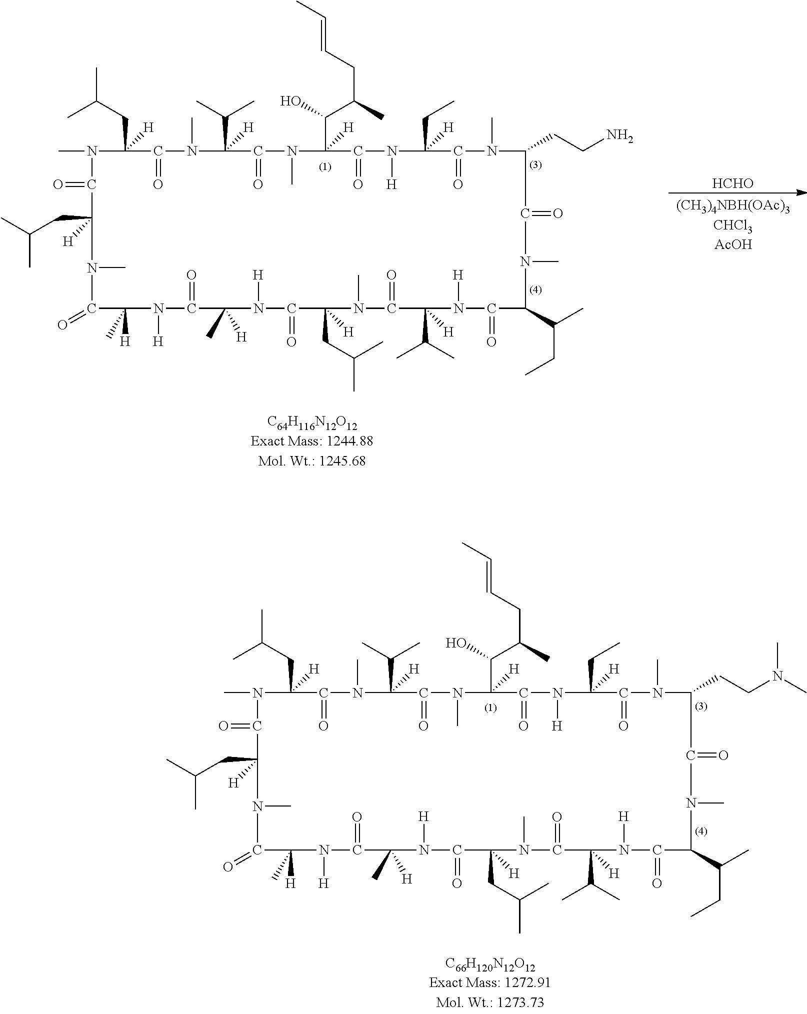 [SMILES: C/C=C/C[C@@H](C)[C@@H](O)[C@H]1C(=O)N[C@@H](CC)C(=O)N(C)[C@H](CCN)C(=O)N(C)[C@@H](C(C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; "HCHO" ; C/C=C/C[C@@H](C)[C@@H](O)[C@H]1C(=O)N[C@@H](CC)C(=O)N(C)[C@H](CCN(C)C)C(=O)N(C)[C@@H](C(C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

[(R)-2-(N,N-Dimethylamino)ethyl-Sar]-3-[NMeIle]-4-cyclosporin is prepared according to a method analogous to that described in Example 20.

### Example 25

### [(R)-2-(N,N-Diethylamino)ethyl-Sar]-3-cyclosporin

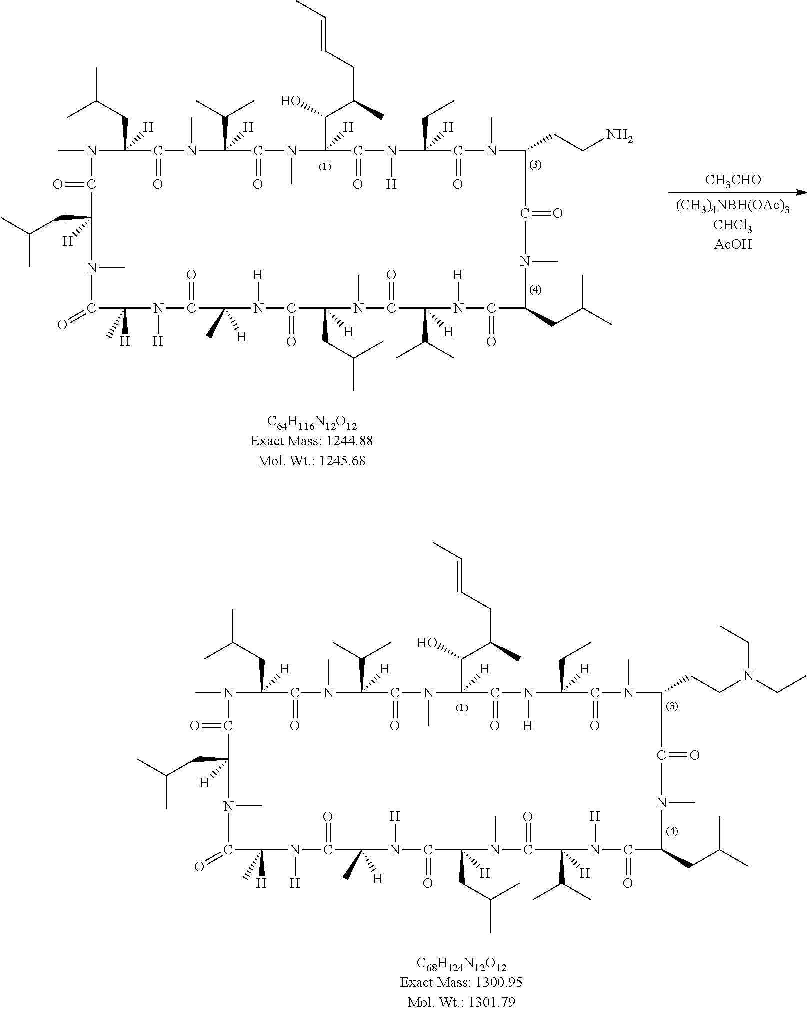

To a solution of [(R)-2-aminoethyl-Sar]-3-cyclosporine (124 mg, 0.10 mmol) in chloroform (6 ml) were added acetaldehyde (FW 44.06, 78 mg, 1.77 mmol) and acetic acid (6 drops). The reaction mixture was stirred at room temperature for 5 minutes. Then tetramethylammonium triacetoxyborohydride (FW 263.10, 126 mg, 0.48 mmol) was added and the reaction mixture was continued to stir for one hour. The mixture was diluted with dichloromethane. The organic layer was washed with aqueous sodium bicarbonate solution and brine, dried over magnesium sulfate, filtered and concentrated. The residue was purified by chromatography (dichloromethane/methanol=97/3) to give product [Molecular formula: C₆₈H₁₂₄N₁₂O₁₂; Exact Mass: 1300.95; MS (m/z): 1301.72 (M+1)⁺; TLC R_{f}: 0.33 (Dichloromethane/methanol=95/5); HPLC RT: 13.28 min. (C8 reverse phase column, 250 mm, acetonitril-water/0.05% TFA, operation temperature 64° C.; Detector: 210 nm)].

### Example 26

### [(R)-2-(N,N-Diethylamino)ethyl-Sar]-3-[(γ-hydroxy)-NMeLeu]-4-cyclosporin

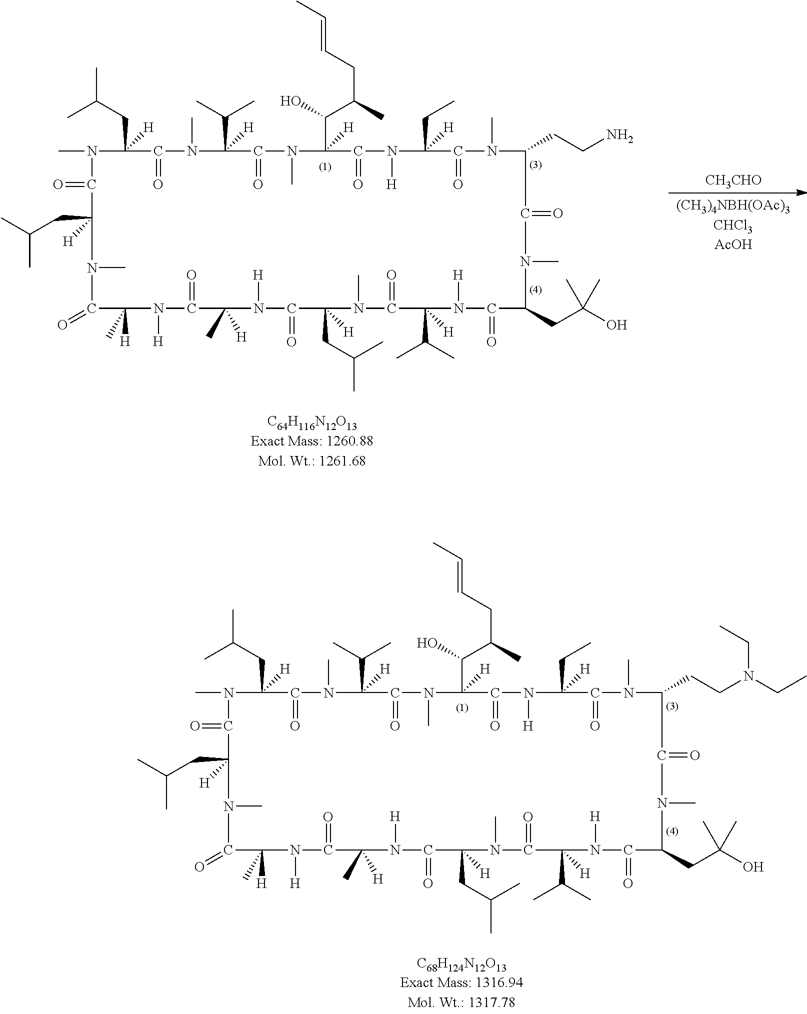 [SMILES: C/C=C/C[C@@H](C)[C@@H](O)[C@H]1C(=O)N[C@@H](CC)C(=O)N(C)[C@H](CCN)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; "CH3CHO" ; C/C=C/C[C@@H](C)[C@@H](O)[C@H]1C(=O)N[C@@H](CC)C(=O)N(C)[C@H](CCN(CC)CC)C(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

To a solution of [(R)-2-aminoethyl-Sar]-3-[(γ-hydroxy)-NMeLeu]-4-cyclosporin (129 mg, 0.10 mmol) in (chloroform 10 ml) were added acetaldehyde (FW 44.06, 80 mg, 1.80 mmol) and acetic acid (7 drops). The reaction mixture was stirred at room temperature for 5 minutes. Then tetramethylammonium triacetoxyborohydride (FW 263.10, 200 mg, 0.76 mmol) was added and the reaction mixture was continued to stir for 1 hour. The mixture was diluted with dichloromethane. The organic layer was washed with aqueous sodium bicarbonate solution and brine, dried over magnesium sulfate, filtered and concentrated. The residue was purified by chromatography (dichloromethane/methanol=95/5) to give the product [Molecular formula: C₆₈H₁₂₄N₁₂O₁₃; Exact Mass: 1316.94; MS (m/z): 1317.70 (M+1)⁺; TLC R_{f}: 0.39 (dichloromethane/methanol=9/1); HPLC RT: 12.06 min. (C8 reverse phase column: 150 mm; acetonitrile/water (0.05% trifluoroacetic acid); operation temperature: 64° C.; detector: 210 nm)].

### Example 27

### [(R)-2-(N,N-Diethylamino)ethyl-Sar]-3-[(γ-methoxy)-NMeLeu]-4-cyclosporin

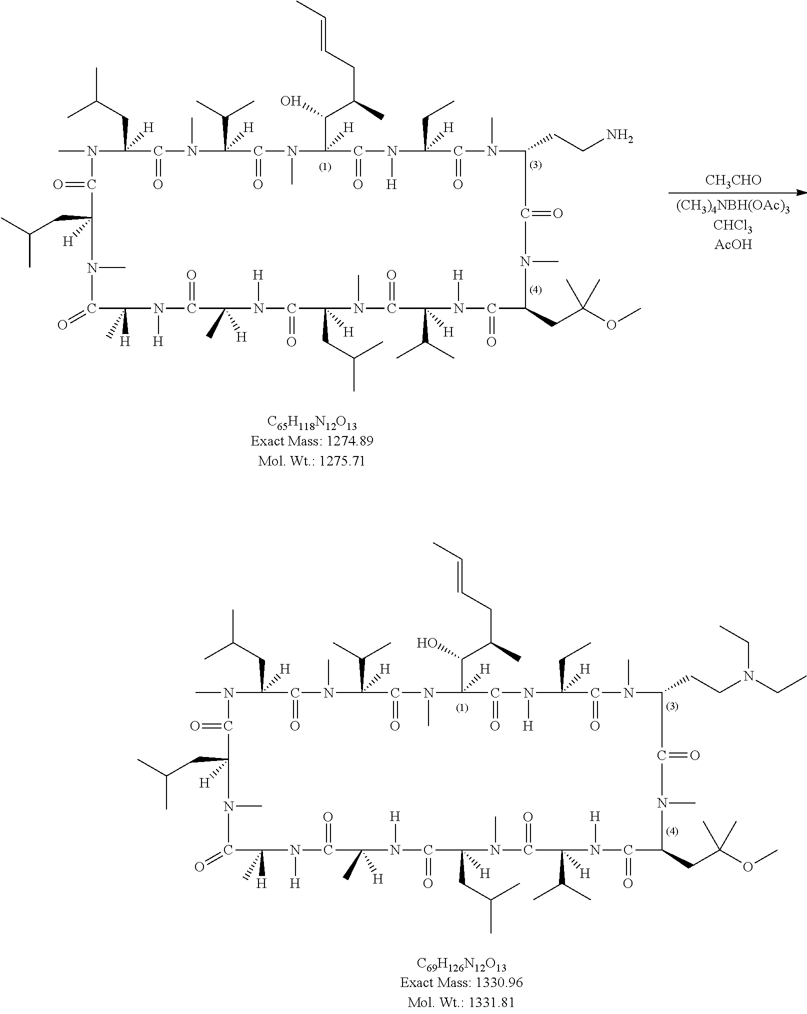

[(R)-2-(N,N-Diethylamino)ethyl-Sar]-3-[(γ-methoxy)-NMeLeu]-4-cyclosporin is prepared according to a method analogous to that described in Example 26.

### Example 28

### [(R)-2-(N,N-Diethylamino)ethyl-Sar]-3-[(γ-allyloxy)-NMeLeu]-4-cyclosporin

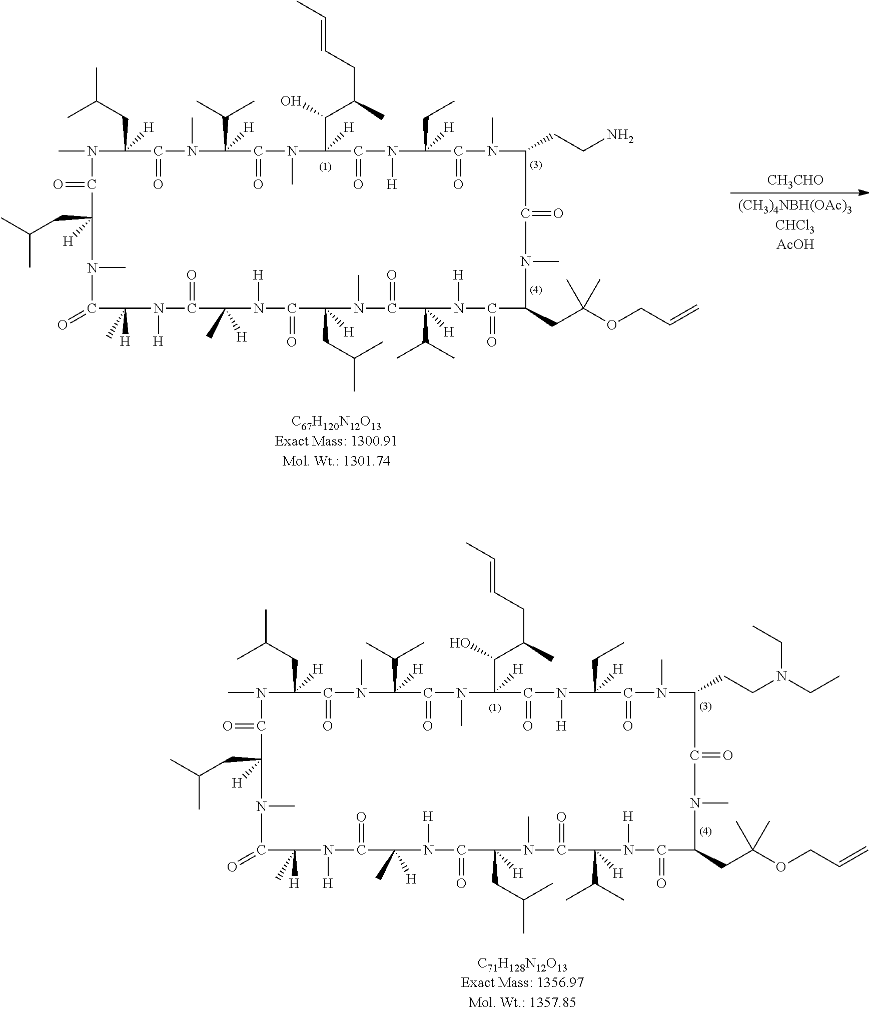

[(R)-2-(N,N-Diethylamino)ethyl-Sar]-3-[(γ-allyloxy)-NMeLeu]-4-cyclosporin is prepared according to a method analogous to that described in Example 26.

### Example 29

### [(R)-2-(N,N-Diethylamino)ethyl-Sar]-3-[NMeVal]-4-cyclosporin

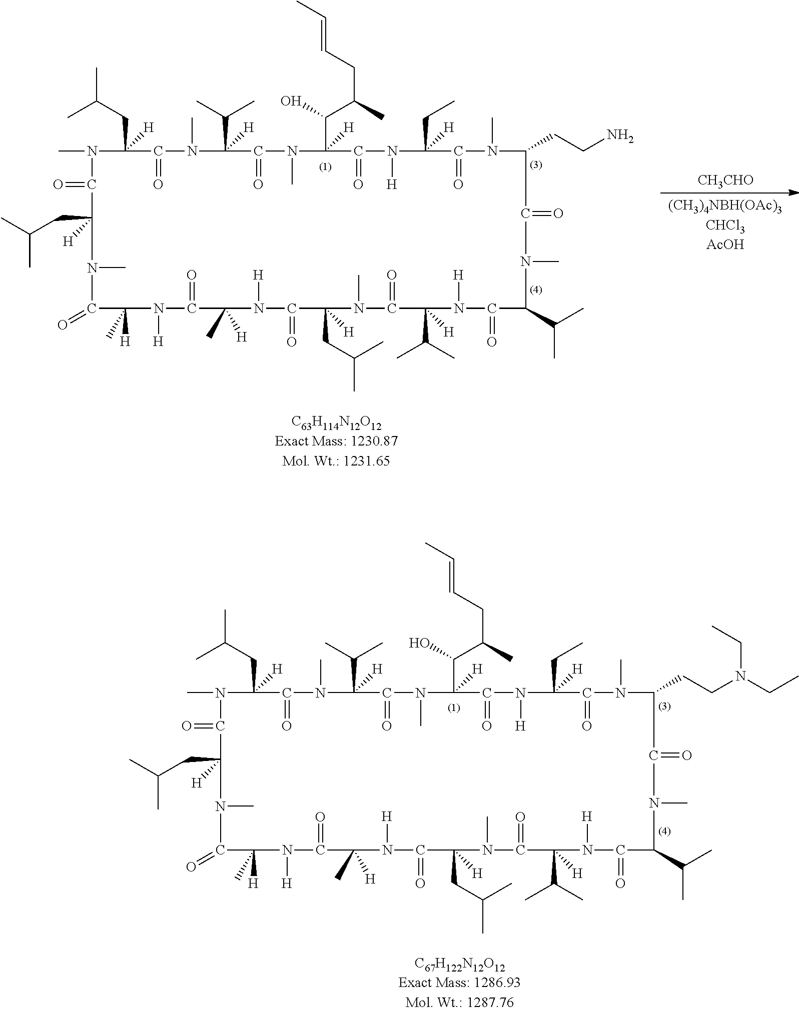 [SMILES: C/C=C/C[C@@H](C)[C@@H](O)[C@H]1C(=O)N[C@@H](CC)C(=O)N(C)[C@H](CCN)C(=O)N(C)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; "CH3CHO" ; C/C=C/C[C@@H](C)[C@@H](O)[C@H]1C(=O)N[C@@H](CC)C(=O)N(C)[C@H](CCN(CC)CC)C(=O)N(C)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

[(R)-2-(N,N-Diethylamino)ethyl-Sar]-3-[NMeVal]-4-cyclosporin is prepared according to a method analogous to that described in Example 26.

### Example 30

### [(R)-2-(N,N-Diethylamino)ethyl-Sar]-3-[NMeIle]-4-cyclosporin

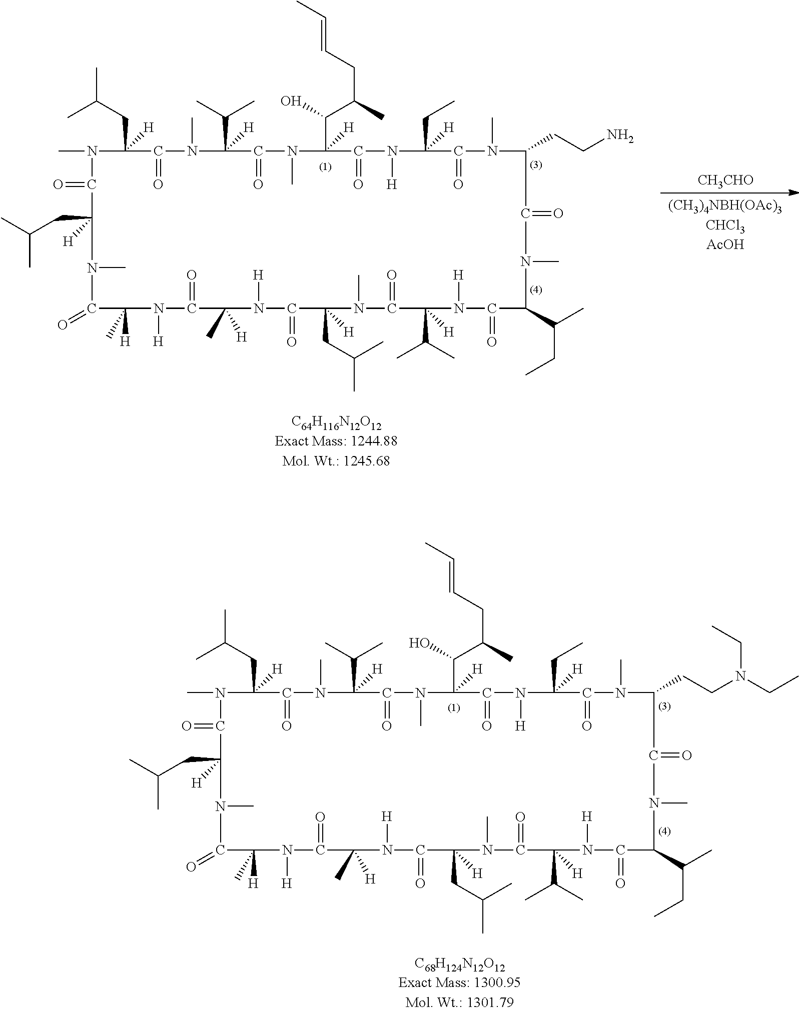 [SMILES: C/C=C/C[C@@H](C)[C@@H](O)[C@H]1C(=O)N[C@@H](CC)C(=O)N(C)[C@H](CCN)C(=O)N(C)[C@@H](C(C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; "CH3CHO" ; C/C=C/C[C@@H](C)[C@@H](O)[C@H]1C(=O)N[C@@H](CC)C(=O)N(C)[C@H](CCN(CC)CC)C(=O)N(C)[C@@H](C(C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

[(R)-2-(N,N-Diethylamino)ethyl-Sar]-3-[NMeIle]-4-cyclosporin is prepared according to a method analogous to that described in Example 26.

### Example 31

### [(S)-2-Aminoethyl-Sar]-3-[(γ-hydroxy)-NMeLeu]-4-cyclosporin

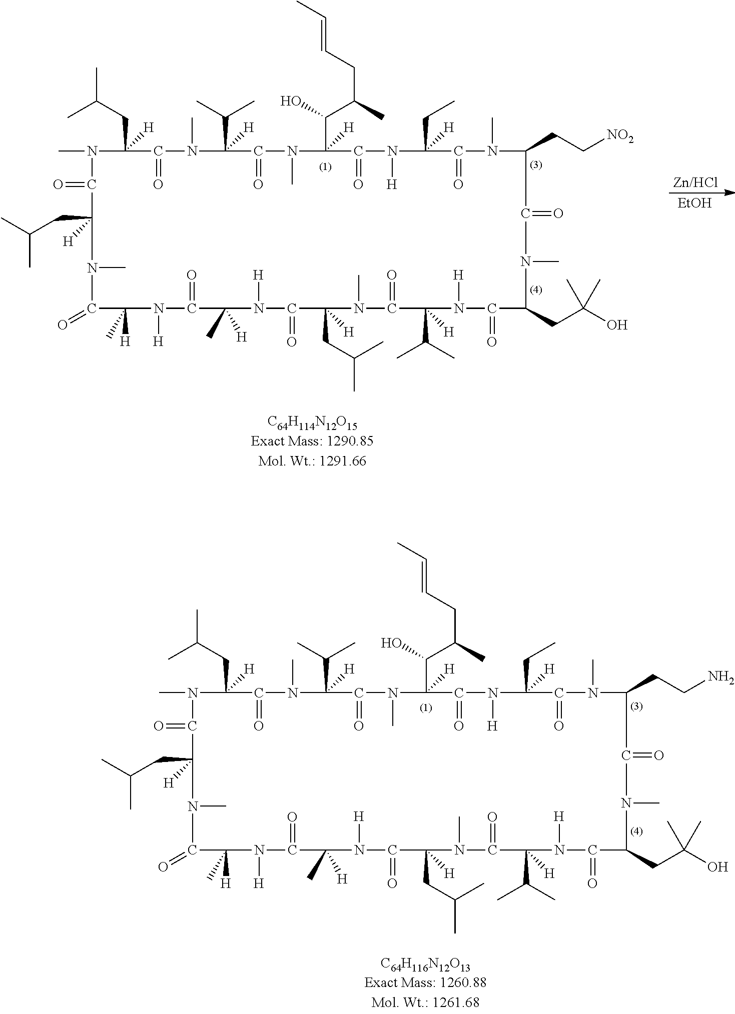

To a mixture of [(S)-2-nitroethyl-Sar]-3-[(γ-hydroxy)-NMeLeu]-4-cyclosporin (350 mg, 0.27 mmol) and zinc (1.50 g) in ethanol (25 ml) was added 10% aqueous hydrochloric acid (15 ml). The reaction mixture was stirred at room temperature overnight (monitored by LC-MS) and filtered. The filter cake was washed with ethanol. The filtrate was concentrated and diluted with dichloromethane. The organic solution was washed with aqueous sodium bicarbonate solution and brine, dried over magnesium sulfate, filtered and concentrated. The residue was purified by chromatography (dichloromethane/methanol=96/4) to give the product [Molecular formula: C₆₄H₁₁₆N₁₂O₁₃; Exact Mass: 1260.88; MS (m/z): 1261.64 (M+1)⁺].

### Example 32

### [(S)-2-(N,N-Dimethylamino)ethyl-Sar]-3-[(γ-hydroxy)-NMeLeu]-4-cyclosporin

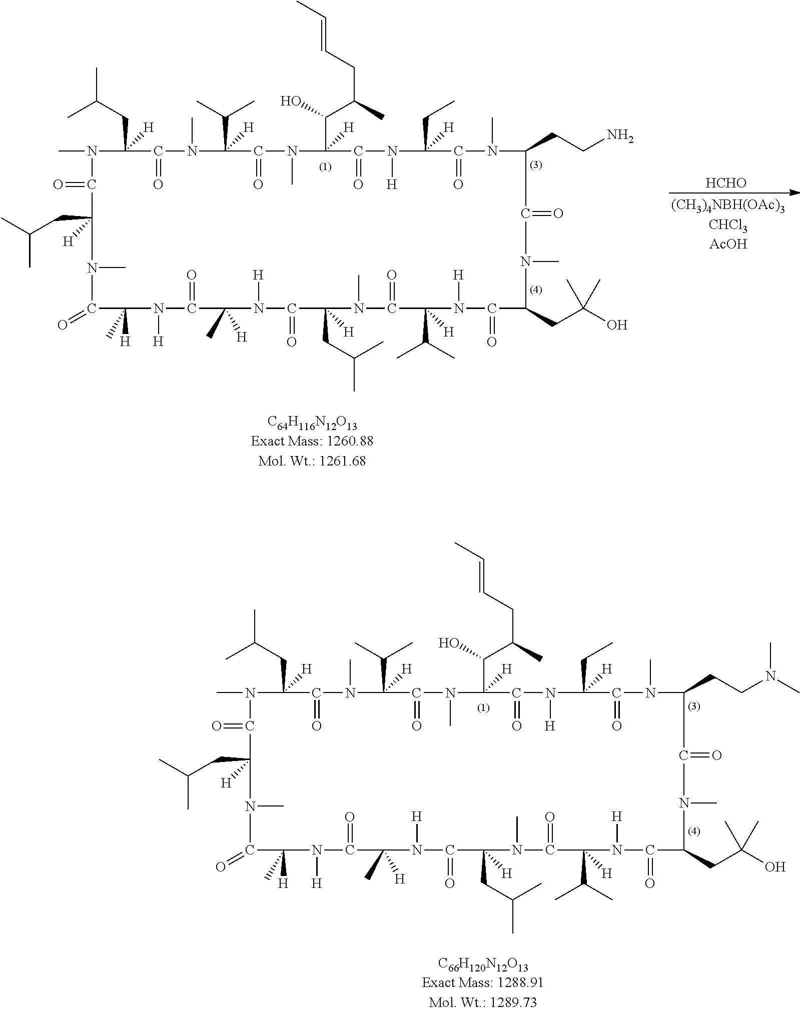

To a solution of [(S)-2-aminoethyl-Sar]-3-[(γ-hydroxy)-NMeLeu]-4-cyclosporin (130 mg, 0.10 mmol) in chloroform (10 ml) were added formaldehyde aqueous 37% solution (0.7 ml) and acetic acid (7 drops). The reaction mixture was stirred at room temperature for 5 minutes. Then tetramethylammonium triacetoxyborohydride (FW 263.10, 200 mg, 0.76 mmol) was added and the reaction mixture was continued to stir for one hour. The mixture was diluted with dichloromethane. The organic layer was washed with aqueous sodium bicarbonate solution and brine, dried over magnesium sulfate, filtered and concentrated. The residue was purified by chromatography (dichloromethane/methanol=95/5) to give the product [Molecular formula: C₆₆H₁₂₀N₁₂O₁₃; Exact Mass: 1288.91; MS (m/z): 1289.70 (M+1)⁺; TLC R_{f}: 0.35 (dichloromethane/methanol=9/1); HPLC RT: 11.02 min. (C8 reverse phase column: 150 mm; acetonitrile/water (0.05% trifluoroacetic acid); operation temperature: 64° C.; detector: 210 nm)].

### Example 33

### [(S)-2-(N,N-Diethylamino)ethyl-Sar]-3-[(γ-hydroxy)-NMeLeu]-4-Cyclosporin

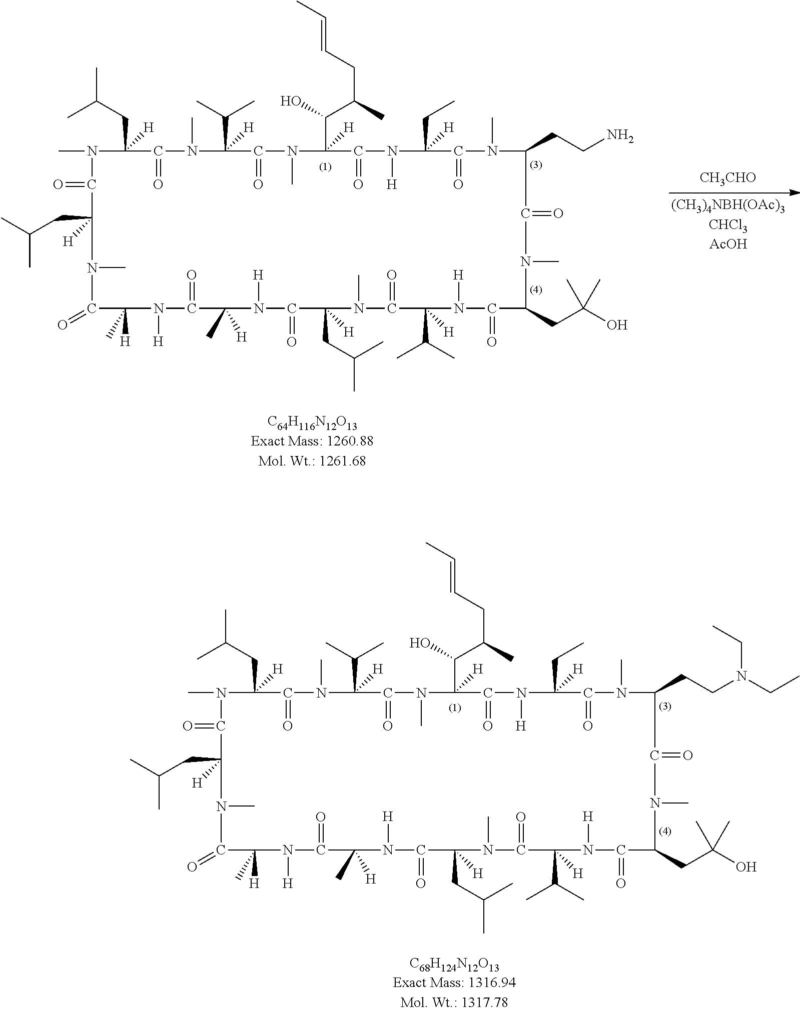

To a solution of [(S)-2-aminoethyl-Sar]-3-[(γ-hydroxy)-NMeLeu]-4-cyclosporin (129 mg, 0.10 mmol) in chloroform (10 ml) were added acetaldehyde (FW 44.06, 80 mg, 1.80 mmol) and acetic acid (7 drops). The reaction mixture was stirred at room temperature for 5 min. Then tetramethylammonium triacetoxyborohydride (FW 263.10, 200 mg, 0.76 mmol) was added and the reaction mixture was continued to stir for one hour. The mixture was diluted with dichloromethane. The organic layer was washed with aqueous sodium bicarbonate solution and brine, dried over magnesium sulfate, filtered and concentrated. The residue was purified by chromatography (dichloromethane/methanol=95/5) to give the product [Molecular formula: C₆₈H₁₂₄N₁₂O₁₃; Exact Mass: 1316.94; MS (m/z): 1317.70 (M+1)⁺; TLC R_{f}: 0.41 (Dichloromethane/methanol=9/1); HPLC RT: 11.96 min. (C8 reverse phase column: 150 mm; acetonitrile/water (0.05% trifluoroacetic acid); operation temperature: 64° C.; detector: 210 nm)].

### Example 34

### [(R)-2-Nitro-3-(N,N-dimethylamino)propyl-Sar]-3-cyclosporin

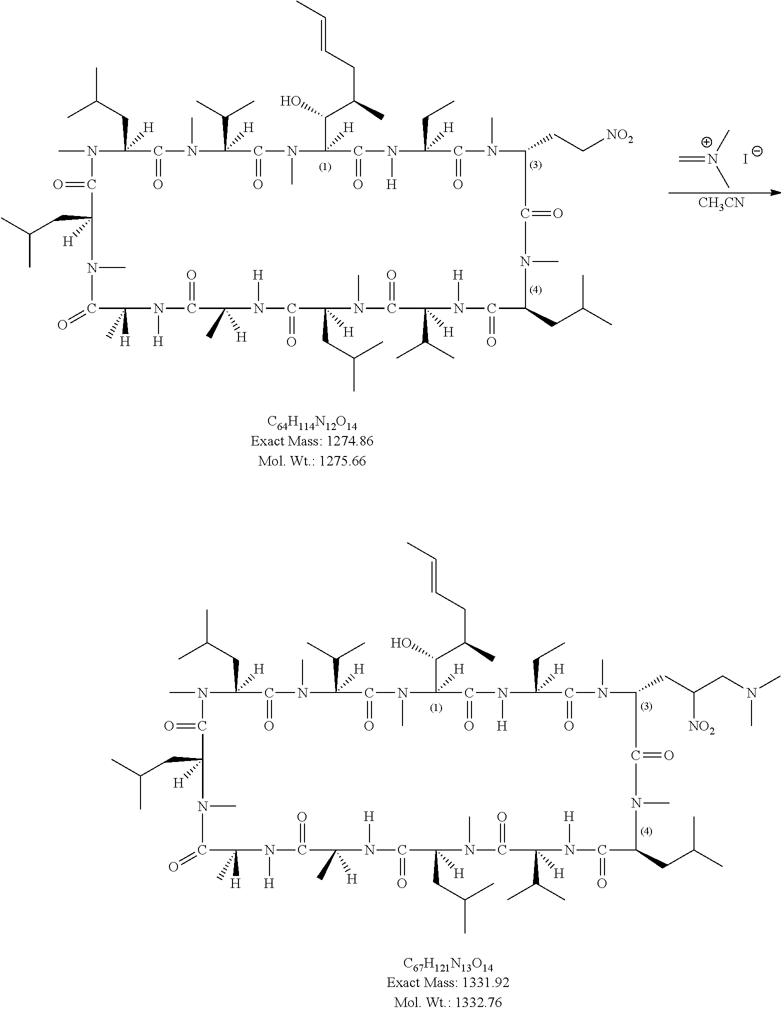

To a mixture of [(R)-2-nitroethyl-Sar]-3-cyclosporin (360 mg, 0.28 mmol) and dimethymethyleneammonium iodide (550 mg, 15.4 mmol) in 25 ml of acetonitrile was added triethylamine (10 drops). The reaction mixture was stirred at room temperature overnight (monitored by LC-MS). The mixture was concentrated and diluted with dichloromethane. The organic layer was washed with aqueous water and brine, dried over magnesium sulfate, filtered and concentrated. The crude product was used for next step.

### Example 35

### [(R)-3-(N,N-Dimethylamino)propyl-Sar]-3-cyclosporin

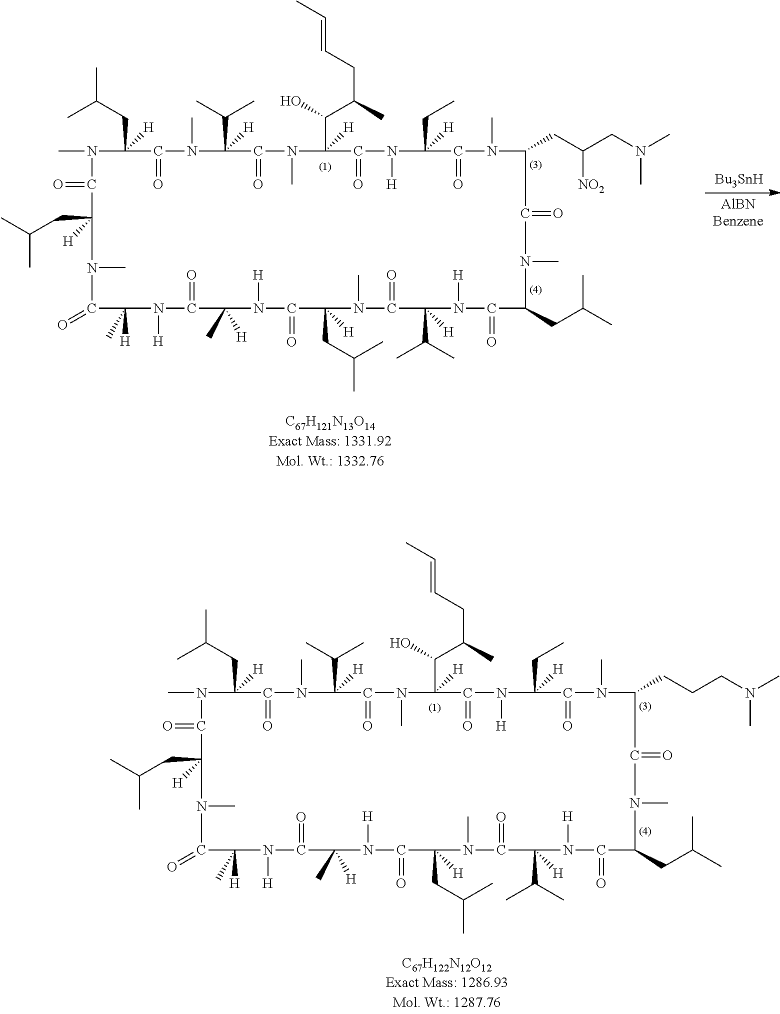

To a mixture of [(R)-2-nitro-3-(N,N-dimethylamino)propyl-Sar]-3-cyclosporin (crude form previous step, 0.28 mmol) and tri-n-butyltin hydride (FW 291.07, 0.41 g, 1.4 mmol) in benzene (25 ml) was added 2,2′-azobisisobutyronitrile (AIBN) (FW 164.21, 46 mg, 0.28 mmol) under nitrogen atmosphere. The mixture was stirred and heated to reflux for 8 hours. After the reaction was completed (monitored by LC-MS), the reaction mixture was washed with aqueous sodium bicarbonate solution and brine followed by evaporated under vacuum. The residue was purified by chromatography (dichloromethane/methanol=95/5) to give the product [Molecular formula: C₆₇H₁₂₂N₁₂O₁₂; Exact Mass: 1286.93; MS (m/z): 1287.71 (M+1)⁺; TLC R_{f}: 0.36 (Dichloromethane/methanol=95/5); HPLC RT: 12.57 min. (C8 reverse phase column: 150 mm; acetonitrile/water (0.05% trifluoroacetic acid); operation temperature: 64° C.; detector: 210 nm)].

### Example 36

### [(R)-2-Nitro-3-(N,N-dimethylamino)propyl-Sar]-3-[(γ-hydroxy)-NMeLeu]-4-cyclosporin

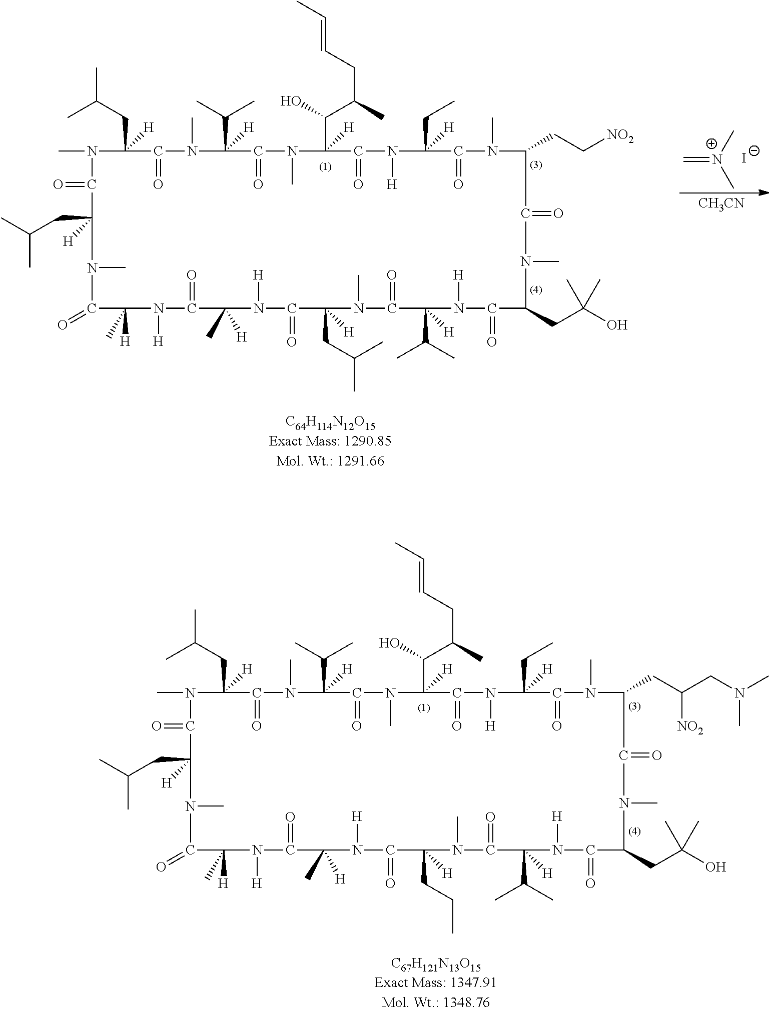

[(R)-2-Nitro-3-(N,N-dimethylamino)propyl-Sar]-3-[(γ-hydroxy)-NMeLeu]-4-cyclosporin is prepared according to a method analogous to that described in Example 34.

### Example 37

### [(R)-3-(N,N-Dimethylamino)propyl-Sar]-3-[(γ-hydroxy)-NMeLeu]-4-cyclosporin

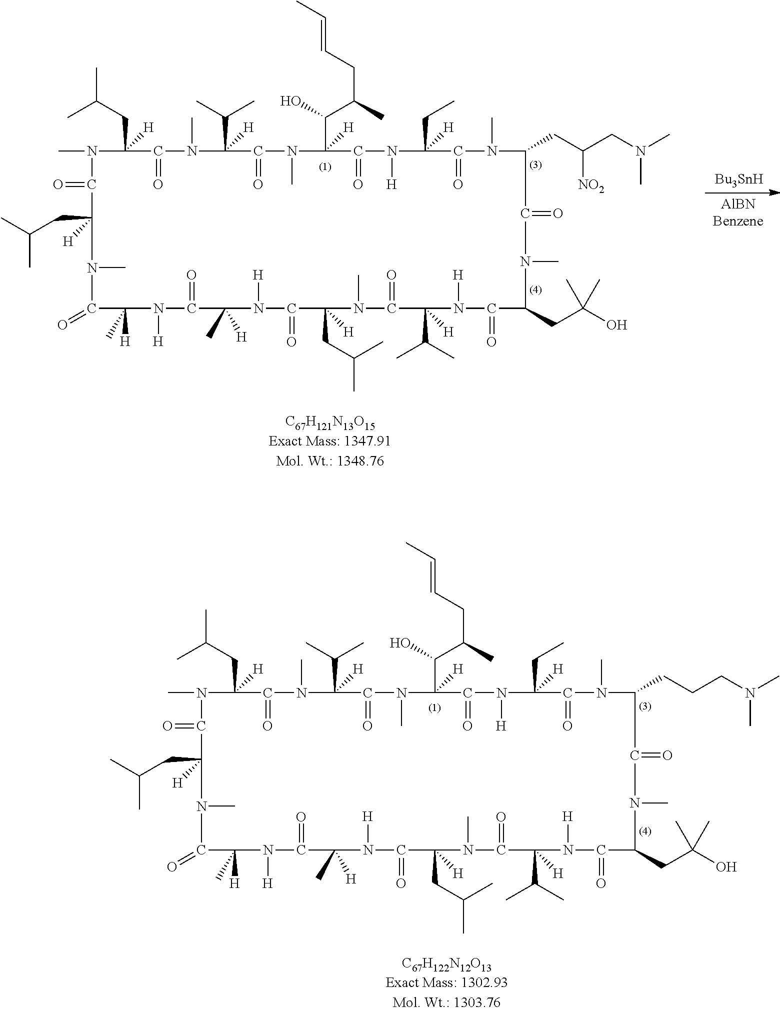

[(R)-3-(N,N-dimethylamino)propyl-Sar]-3-[(γ-hydroxy)-NMeLeu]-4-cyclosporin is prepared according to a method analogous to that described in Example 35.

### Example 38

### [(R)-2-Nitro-3-(N,N-dimethylamino)propyl-Sar]-3-[(γ-methoxy)-N-MeLeu]-4-cyclosporin

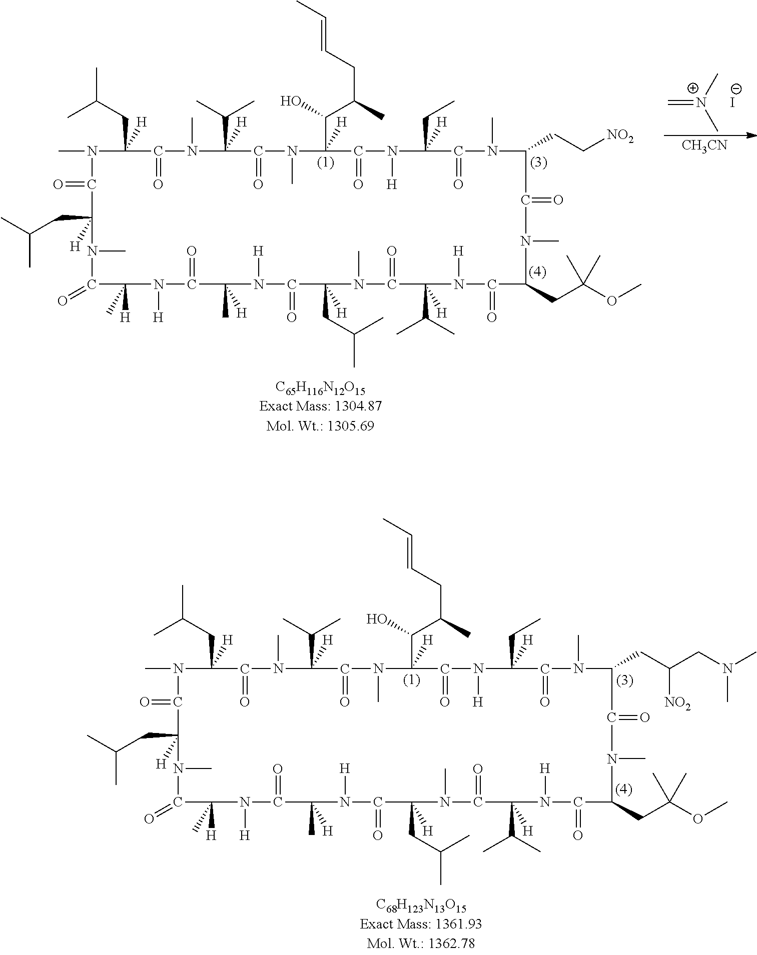

[(R)-2-Nitro-3-(N,N-dimethylamino)propyl-Sar]-3-[(γ-methoxy)-NMeLeu]-4-cyclosporin is prepared according to a method analogous to that described in Example 34.

### Example 39

### [(R)-3-(N,N-Dimethylamino)propyl-Sar]-3-[(γ-methoxy)-NMeLeu]-4-cyclosporin

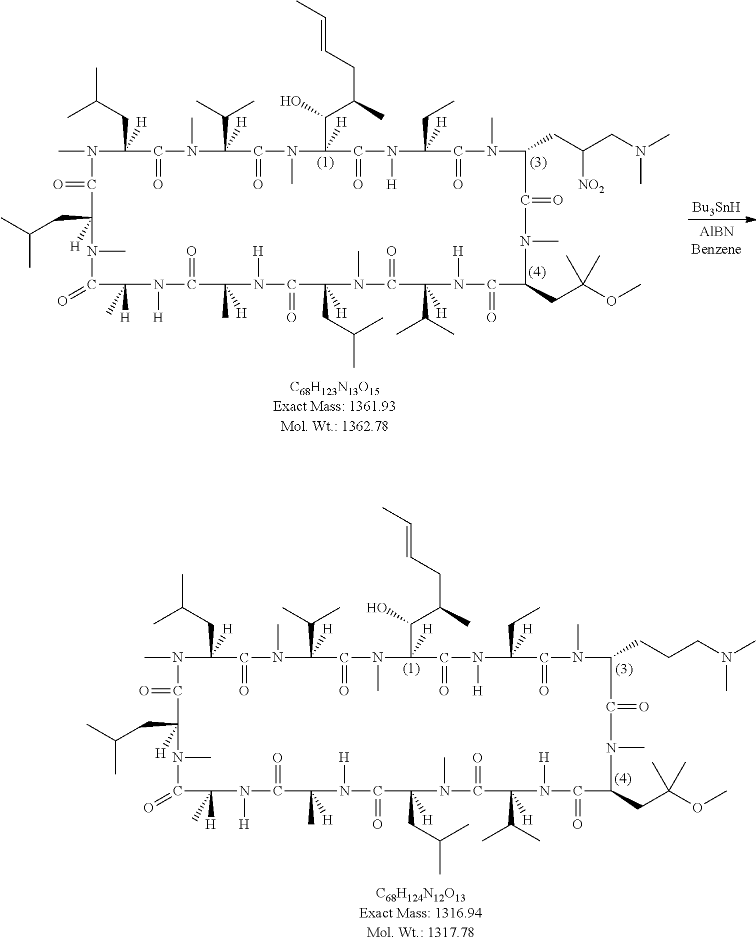

[(R)-3-(N,N-dimethylamino)propyl-Sar]-3-[(γ-methoxy)-NMeLeu]-4-cyclosporin is prepared according to a method analogous to that described in Example 35.

### Example 40

### [(R)-2-Nitro-3-(N,N-dimethylamino)propyl-Sar]-3-[(γ-allyloxy)-NMeLeu]-4-cyclosporin

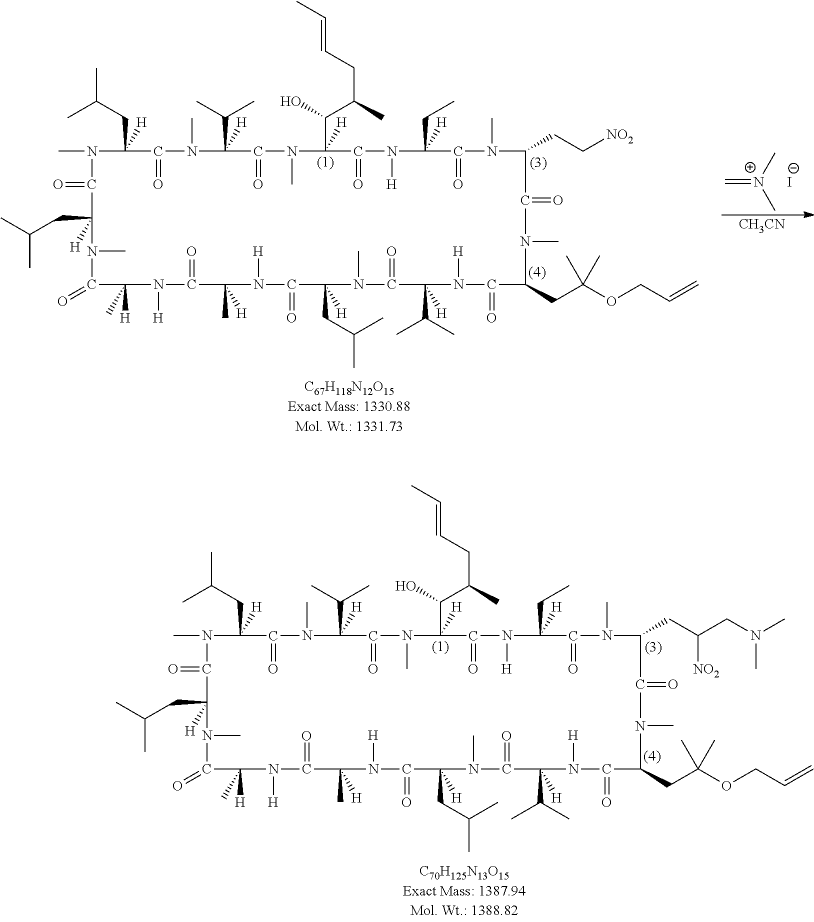

[(R)-2-Nitro-3-(N,N-dimethylamino)propyl-Sar]-3-[(γ-allyloxy)-NMeLeu]-4-cyclosporin is prepared according to a method analogous to that described in Example 34.

### Example 41

### [(R)-3-(N,N-Dimethylamino)propyl-Sar]-3-[(γ-allyloxy)-NMeLeu]-4-cyclosporin

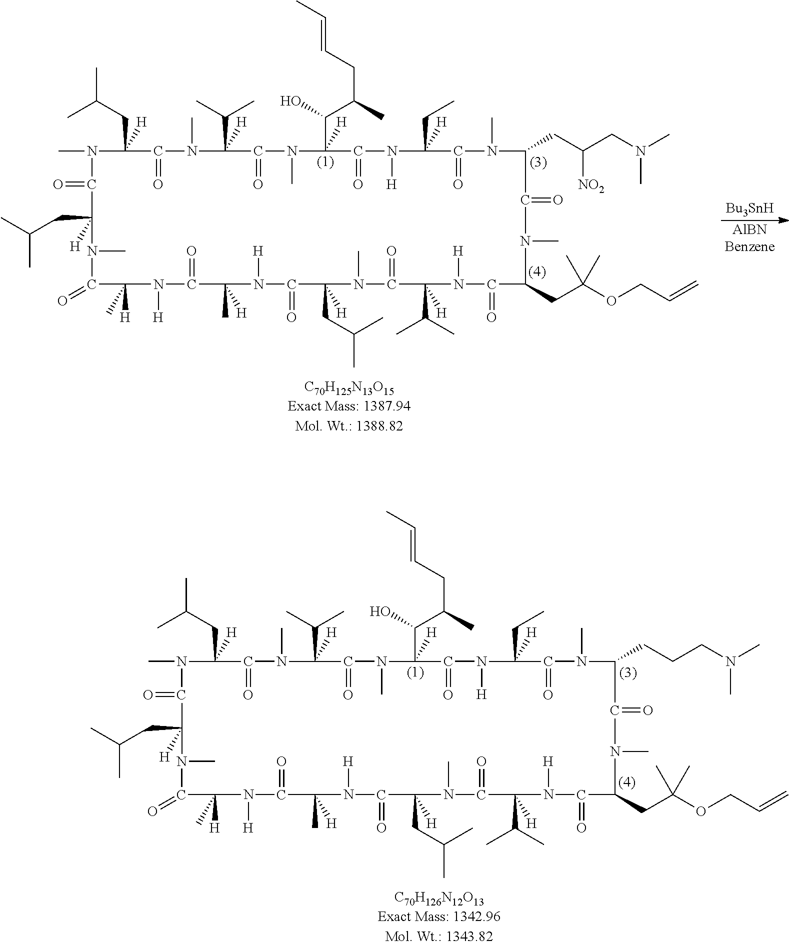

[(R)-3-(N,N-dimethylamino)propyl-Sar]-3-[(γ-allyloxy)-NMeLeu]-4-cyclosporin is prepared according to a method analogous to that described in Example 35.

### Example 42

### [(R)-2-Nitro-3-(N,N-dimethylamino)propyl-Sar]-3-[NMeVal]-4-cyclosporin

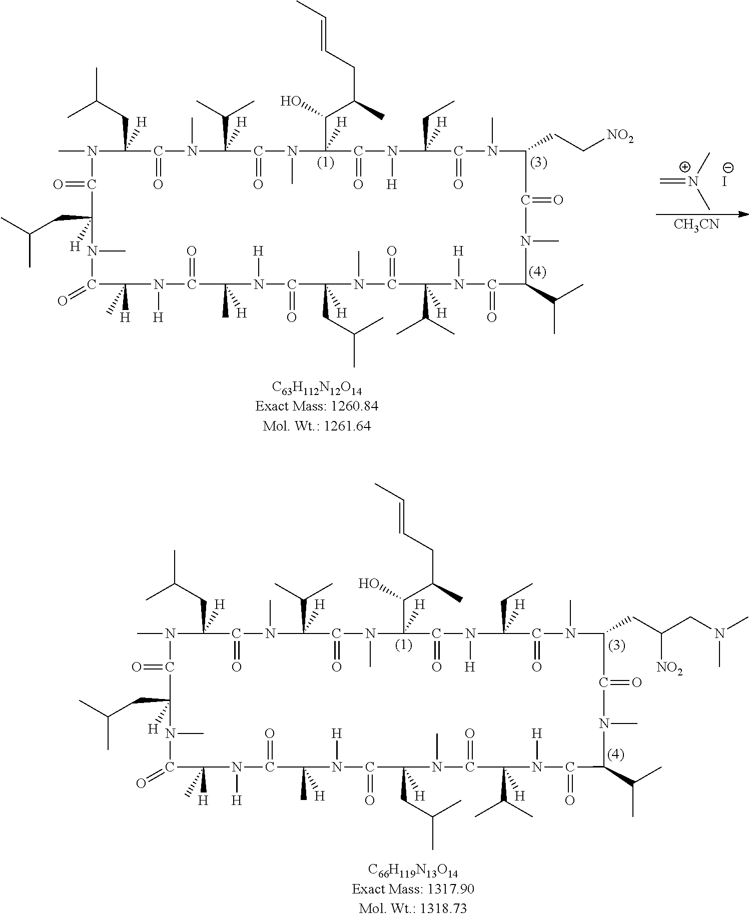

[(R)-2-Nitro-3-(N,N-dimethylamino)propyl-Sar]-3-[NMeVal]-4-cyclosporin is prepared according to a method analogous to that described in Example 34.

### Example 43

### [(R)-3-(N,N-Dimethylamino)propyl-Sar]-3-[NMeVal]-4-cyclosporin

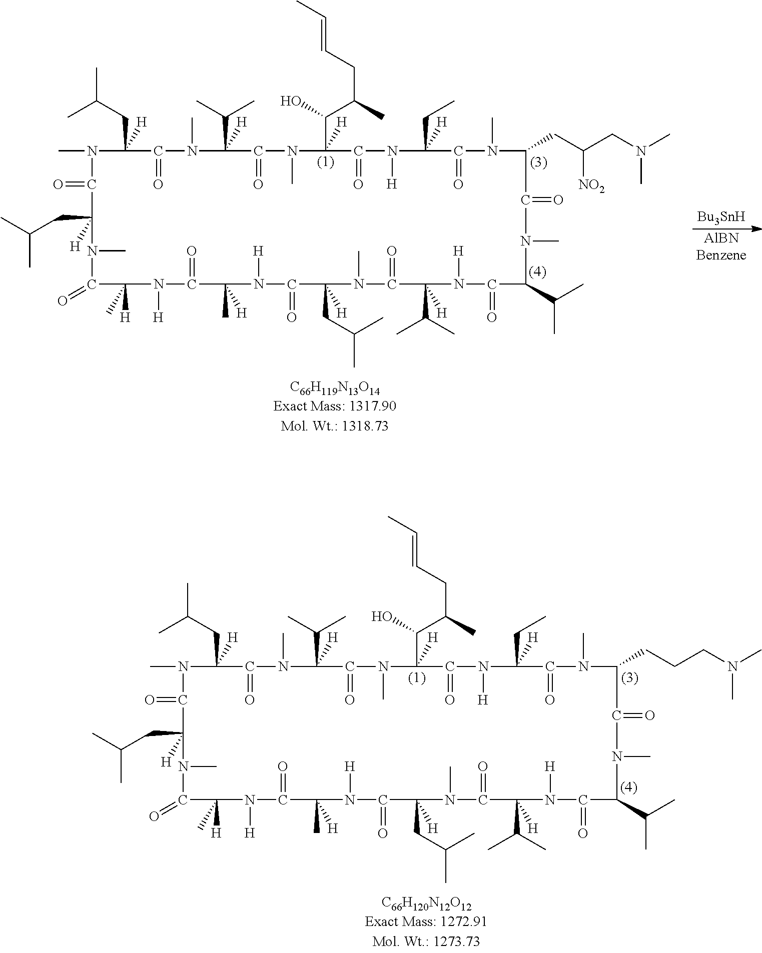

[(R)-3-(N,N-dimethylamino)propyl-Sar]-3-[NMeVal]-4-cyclosporin is prepared according to a method analogous to that described in Example 35.

### Example 44

### [(R)-2-Nitro-3-(N,N-dimethylamino)propyl-Sar]-3-[NMeIle]-4-cyclosporin

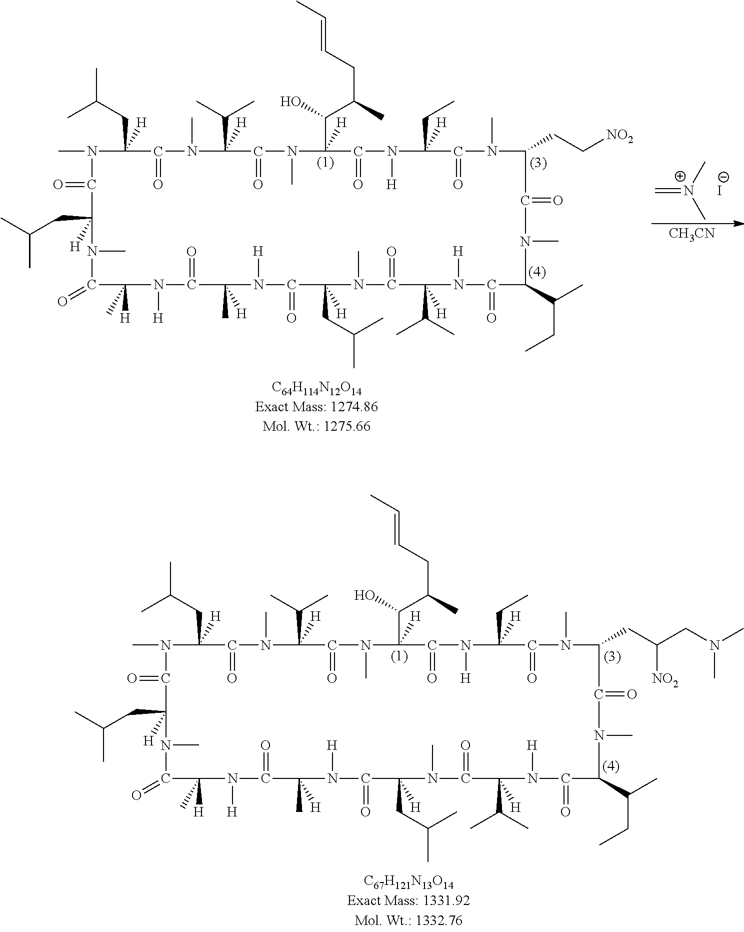

[(R)-2-Nitro-3-(N,N-dimethylamino)propyl-Sar]-3-[NMeIle]-4-cyclosporin is prepared according to a method analogous to that described in Example 34.

### Example 45

### [(R)-3-(N,N-Dimethylamino)propyl-Sar]-3-[NMeIle]-4-cyclosporin

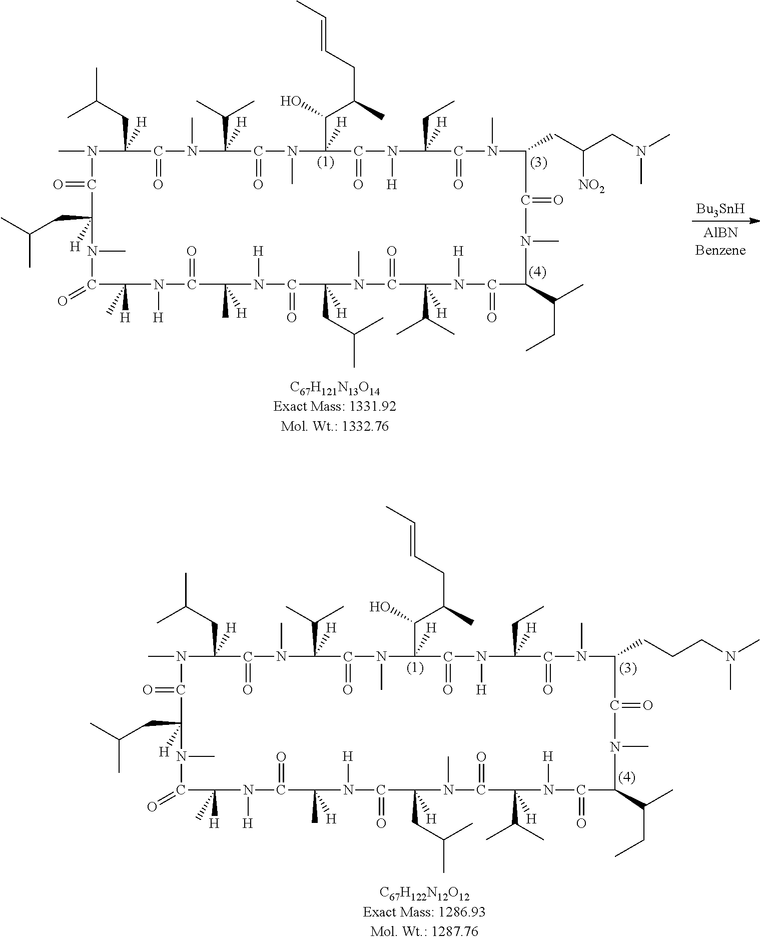

[(R)-3-(N,N-dimethylamino)propyl-Sar]-3-[NMeIle]-4-cyclosporin is prepared according to a method analogous to that described in Example 35.

### Reference Example 1

### [α-Carboxy-Sar]-3-cyclosporin

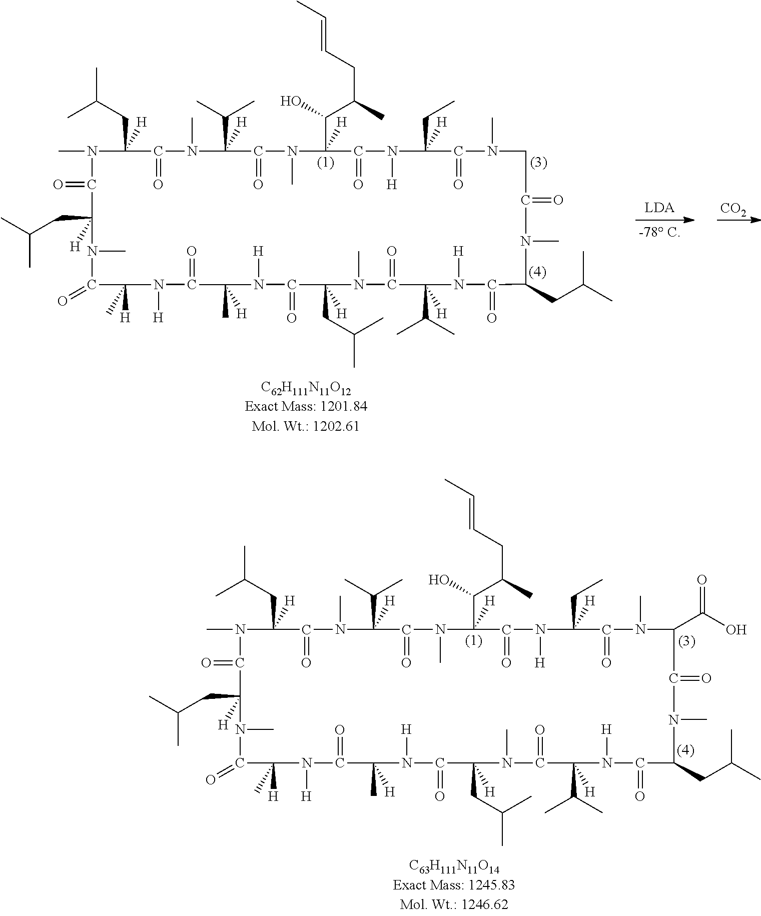

n-Butyllithium (2.87 M, 27 mmol, 9.4 ml, 10 eq) was added to a solution of diisopropylamine (3.8 ml, 27 mmol, 10 eq) in tetrahydrofuran (80 ml) at −78° C. under nitrogen. After the reaction mixture was stirred for an hour, a solution of cyclosporine (3.20 g, 2.66 mmol) in tetrahydrofuran (15 ml) was added over 10 minutes. The mixture was stirred at −78° C. for 2 hours. Carbon dioxide gas was bubbled through the reaction mixture for 20-25 minutes and the mixture was stirred at −78° C. for another hour. Then the cooling bath was removed and the reaction mixture was allowed to warm up to 0° C. slowly. Most of tetrahydrofuran was removed under vacuum at room temperature. The residue was quenched by the addition of saturated citric acid solution and the pH of the mixture was adjusted to around 7-8. The unreacted cyclosporin was extracted with ether (40 ml×2). The PH of the aqueous layer was adjusted to 3-4 with 1 N hydrochloric acid and the precipitated oil was extracted with ethyl acetate (100 ml). The aqueous layer was extracted with ethyl acetate (100 ml×3). The combined ethyl acetate layers were washed with brine, dried over magnesium sulfate and evaporated under reduced pressure to give semi-solid product (2.61 g, yield: 78%) [Molecular Formula: C₆₃H₁₁₁N₁₁O₁₄; Exact Mass: 1245.83; MS (m/z): 1246.7 (M+1)⁺, 1268.7 (M+Na)⁺].

[α-Carboxy-Sar]-3-cyclosporin was prepared according to a procedure described by Seebach D, et al., 1993, Helv Chim Acta, 76, 1564-1590.

### Reference Example 2

### [α-Methoxycarbonyl-Sar]-3-cyclosporin

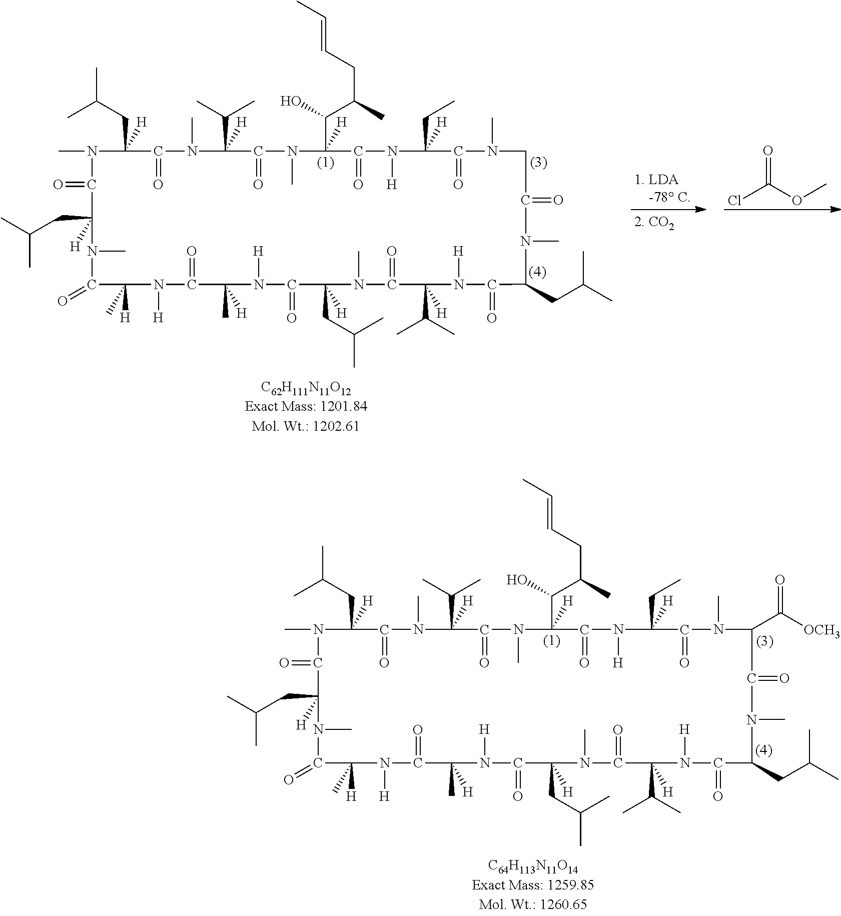

An alternative synthesis of [α-methoxycarbonyl-Sar]-3-cyclosporin was described by Seebach D, et al., 1993, Helv Chim Acta, 76, 1564-1590.

### Reference Example 3

### [(R)-α-(Hydroxymethyl)-Sar]-3-cyclosporin

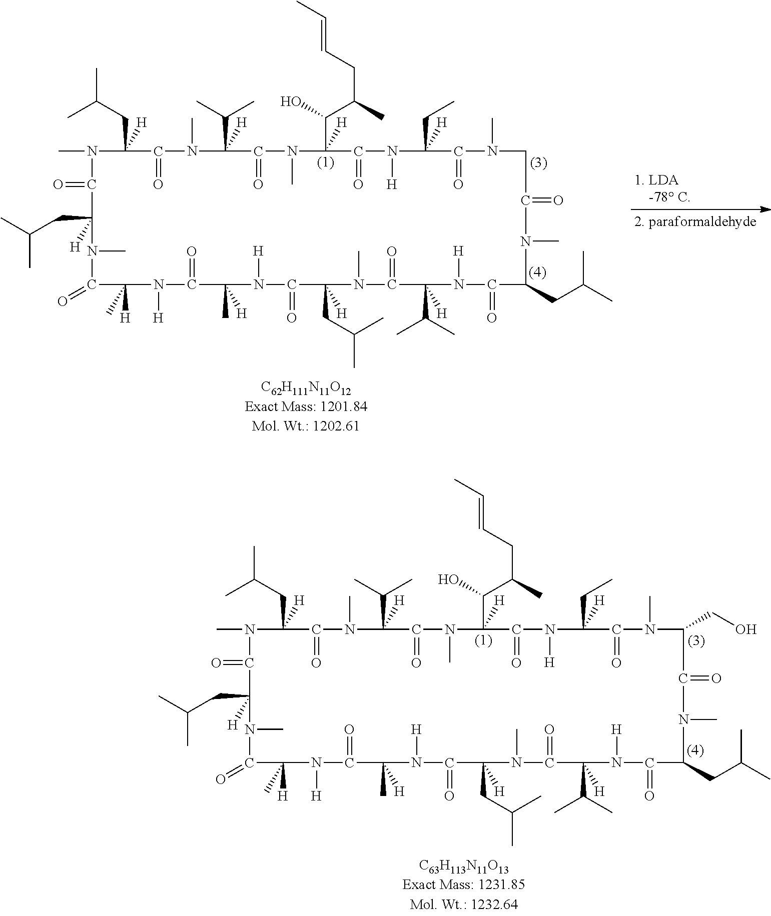

An alternative synthesis of [(R)-α-(hydroxymethyl)-Sar]-3-cyclosporin was described by Seebach D, et al., 1993, Helv Chim Acta, 76, 1564-1590.

### Reference Example 4

### [(γ-Methoxy)-N-MeLeu]-4-cyclosporin

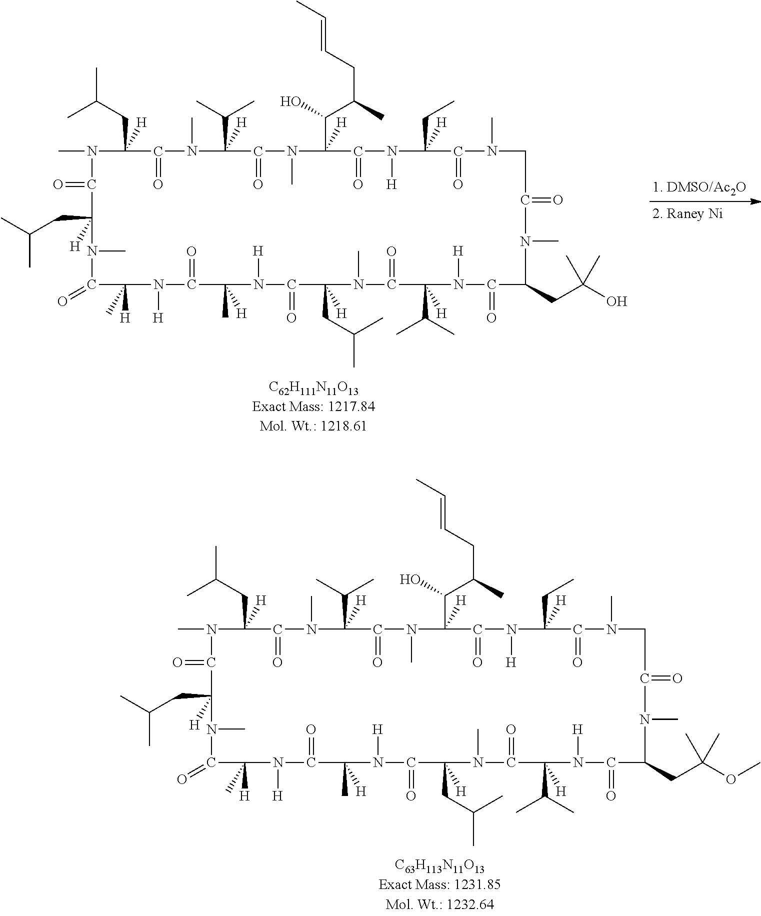 [SMILES: C/C=C/C[C@@H](C)[C@@H](O)[C@H]1C(=O)N[C@@H](CC)C(=O)N(C)CC(=O)N(C)[C@@H](CC(C)(C)O)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C ; C/C=C/C[C@@H](C)[C@@H](O)[C@H]1C(=O)N[C@@H](CC)C(=O)N(C)CC(=O)N(C)[C@@H](CC(C)(C)OC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

The synthesis of [(γ-methoxy)-NMeLeu]-4-cyclosporin was described by Su, Z. et al. in WO2012/021796 and WO2012/075494.

### Reference Example 5

### [(γ-Allyloxy)-NMeLeu]-4-cyclosporin

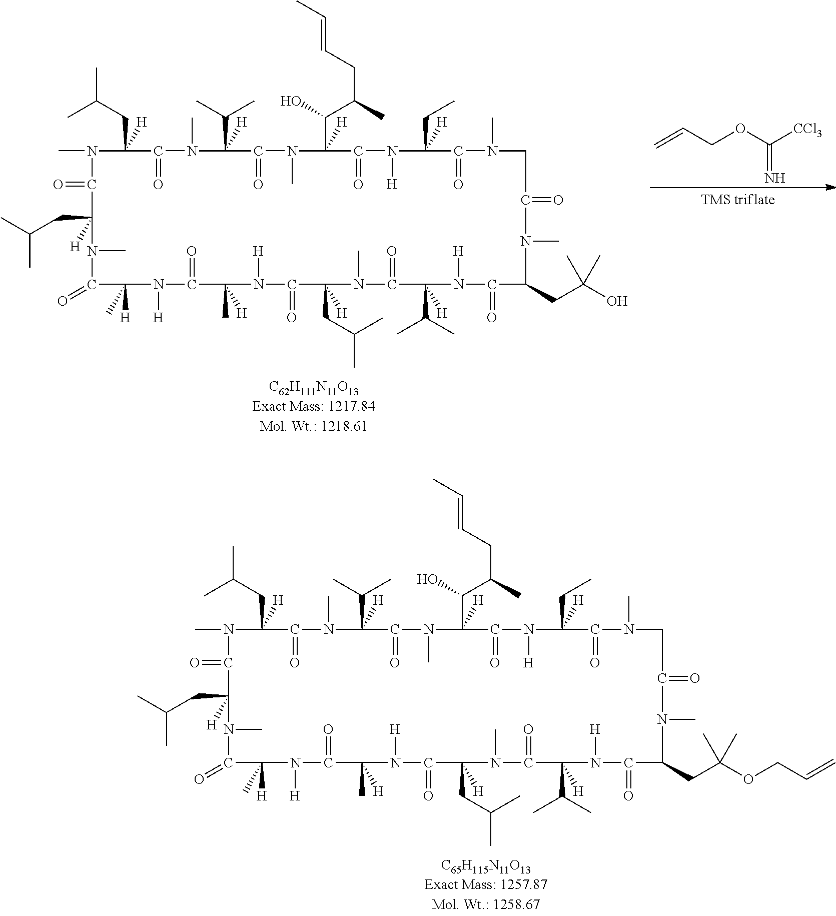

The synthesis of [(γ-allyloxy)-NMeLeu]-4-cyclosporin was described by Su, Z. et al. in WO2012/021796.

### Reference Example 6

### [NMeVal]-4-cyclosporin

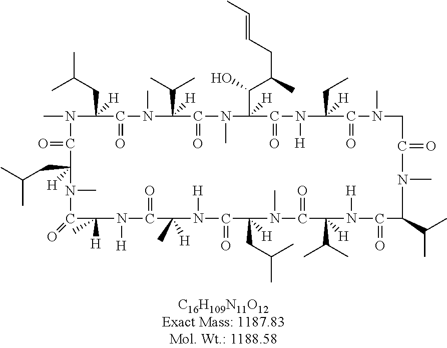 [SMILES: C/C=C/C[C@@H](C)[C@@H](O)[C@H]1C(=O)N[C@@H](CC)C(=O)N(C)CC(=O)N(C)[C@@H](C(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

The synthesis of [NMeVal]-4-cyclosporin has been described by Su, Z. et al. in WO2012/009715 and WO2012/075494.

### Reference Example 7

### [NMeIle]-4-cyclosporin

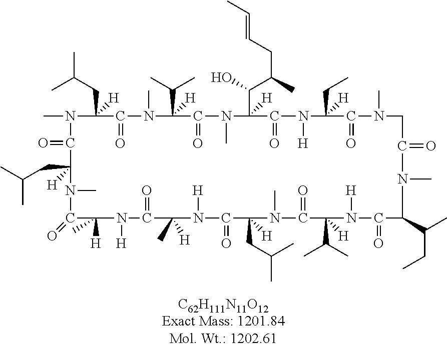 [SMILES: C/C=C/C[C@@H](C)[C@@H](O)[C@H]1C(=O)N[C@@H](CC)C(=O)N(C)CC(=O)N(C)[C@@H](C(C)CC)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

The synthesis of [NMeIle]-4-cyclosporin was described by Su, Z. et al. in WO2012/009715 and WO2012/075494.

### Examples 46-237

### Cyclosporin Derivatives

The following compounds can be prepared according to a method analogous to those described herein.

TABLE 1

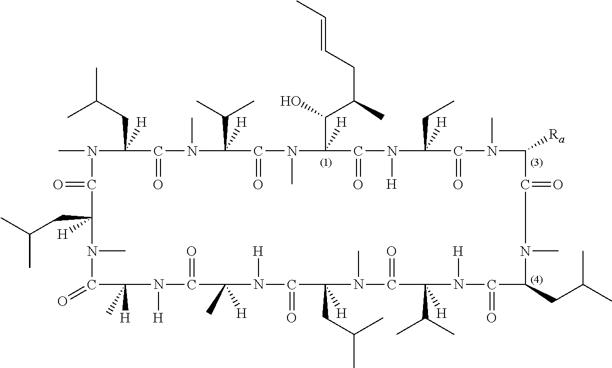

| Ex. | | |
|---|---|---|
| No. | Rₐ | Name |
| 46 | 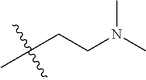 [SMILES: *CCN(C)C] | [(R)-2-(N,N-Dimethylamino)ethyl-Sar]-3-cyclosporin |
| 47 | 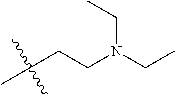 [SMILES: *CCN(CC)CC] | [(R)-2-(N,N-Diethylamino)ethyl-Sar]-3-cyclosporin |
| 48 | 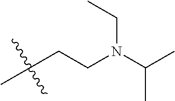 [SMILES: *CCN(CC)C(C)C] | [(R)-2-(N-Ethyl-N-isopropylamino)ethyl-Sar]-3-cyclosporin |
| 49 | 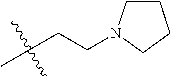 [SMILES: *CCN1CCCC1] | [(R)-2-(N-Pyrrolidinyl)ethyl-Sar]-3-cyclosporin |
| 50 | 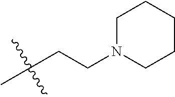 [SMILES: *CCN1CCCCC1] | [(R)-2-(N-Piperidinyl)ethyl-Sar]-3-cyclosporin |
| 51 | 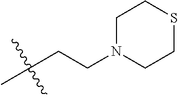 [SMILES: *CCN1CCSCC1] | [(R)-2-(N-Thiomorpholino)ethyl-Sar]-3-cyclosporin |
| 52 | 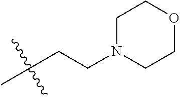 [SMILES: *CCN1CCOCC1] | [(R)-2-(N-Morpholino)ethyl-Sar]-3-cyclosporin |
| 53 | 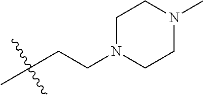 [SMILES: *CCN1CCN(C)CC1] | [(R)-2-(N-4-Methylpiperazinyl)ethyl-Sar]-3-cyclosporin |
| 53a | 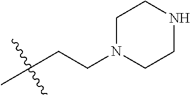 [SMILES: *CCN1CCNCC1] | [(R)-2-(N-4-Piperazinyl)ethyl-Sar]-3-cyclosporin |
| 54 | 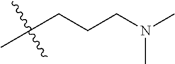 [SMILES: *CCCN(C)C] | [(R)-3-(N,N-Dimethylamino)propyl-Sar]-3-cyclosporin |
| 55 | 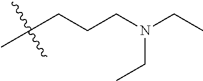 [SMILES: *CCCN(CC)CC] | [(R)-3-(N,N-Diethylamino)propyl-Sar]-3-cyclosporin |
| 56 | 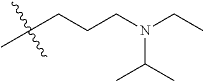 [SMILES: *CCCN(CC)C(C)C] | [(R)-3-(N-Ethyl-N-isopropylamino)propyl-Sar]-3- cyclosporin |
| 57 | 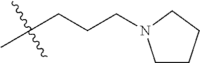 [SMILES: *CCCN1CCCC1] | [(R)-3-(N-Pyrrolidinyl)propyl-Sar]-3-cyclosporin |
| 58 | 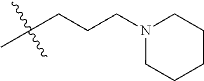 [SMILES: *CCCN1CCCCC1] | [(R)-3-(N-Piperidinyl)propyl-Sar]-3-cyclosporin |
| 59 | 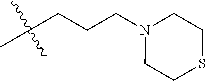 [SMILES: *CCCN1CCSCC1] | [(R)-3-(N-Thiomorpholino)propyl-Sar]-3-cyclosporin |
| 60 | 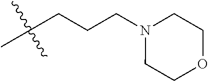 [SMILES: *CCCN1CCOCC1] | [(R)-3-(N-Morpholino)propyl-Sar]-3-cyclosporin |
| 61 | 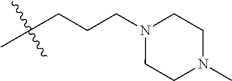 [SMILES: *CCCN1CCN(C)CC1] | [(R)-3-(N-4-Methylpiperazinyl)propyl-Sar]-3-cyclosporin |
| 61a | 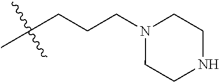 [SMILES: *CCCN1CCNCC1] | [(R)-3-(N-4-Piperazinyl)propyl-Sar]-3-cyclosporin |
| 62 | 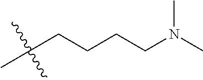 [SMILES: *CCCCN(C)C] | [(R)-4-(N,N-Dimethylamino)butyl-Sar]-3-cyclosporin |
| 63 | 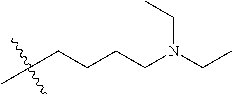 [SMILES: *CCCCN(CC)CC] | [(R)-4-(N,N-Diethylamino)butyl-Sar]-3-cyclosporin |
| 64 | 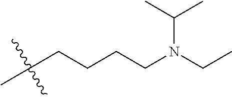 [SMILES: *CCCCN(CC)C(C)C] | [(R)-4-(N-Ethyl-N-isopropylamino)butyl-Sar]-3-cyclosporin |
| 65 | 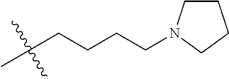 [SMILES: *CCCCN1CCCC1] | [(R)-4-(N-Pyrrolidinyl)butyl-Sar]-3-cyclosporin |
| 66 | 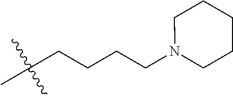 [SMILES: *CCCCN1CCCCC1] | [(R)-4-(N-Piperidinyl)butyl-Sar]-3-cyclosporin |
| 67 | 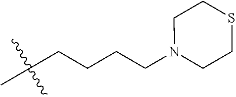 [SMILES: *CCCCN1CCSCC1] | [(R)-4-(N-Thiomorpholino)butyl-Sar]-3-cyclosporin |
| 68 | 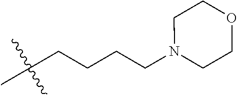 [SMILES: *CCCCN1CCOCC1] | [(R)-4-(N-Morpholino)butyl-Sar]-3-cyclosporin |
| 69 | 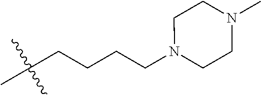 [SMILES: *CCCCN1CCN(C)CC1] | [(R)-4-(N-4-Methylpiperazinyl)butyl-Sar]-3-cyclosporin |
| 69a | 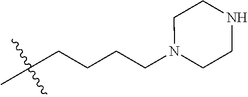 [SMILES: *CCCCN1CCNCC1] | [(R)-4-(N-4-Piperazinyl)butyl-Sar]-3-cyclosporin |
| 70 | 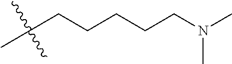 [SMILES: *CCCCCN(C)C] | [(R)-5-(N,N-Dimethylamino)pentyl-Sar]-3-cyclosporin |
| 71 | 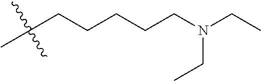 [SMILES: *CCCCCN(CC)CC] | [(R)-5-(N,N-Diethylamino)pentyl-Sar]-3-cyclosporin |
| 72 | 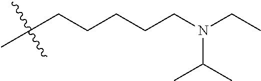 [SMILES: *CCCCCN(CC)C(C)C] | [(R)-5-(N-Ethyl-N-isopropylamino)pentyl-Sar]-3- cyclosporin |
| 73 | 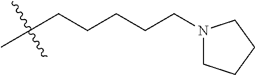 [SMILES: *CCCCCN1CCCC1] | [(R)-5-(N-Pyrrolidinyl)pentyl-Sar]-3-cyclosporin |
| 74 | 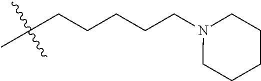 [SMILES: *CCCCCN1CCCCC1] | [(R)-5-(N-Piperidinyl)pentyl-Sar]-3-cyclosporin |
| 75 | 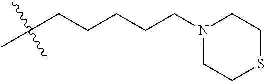 [SMILES: *CCCCCN1CCSCC1] | [(R)-5-(N-Thiomoipholino)pentyl-Sar]-3-cyclosporin |
| 76 | 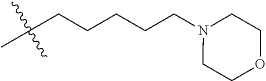 [SMILES: *CCCCCN1CCOCC1] | [(R)-5-(N-Morpholino)pentyl-Sar]-3-cyclosporin |
| 77 | 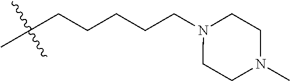 [SMILES: *CCCCCN1CCN(C)CC1] | [(R)-5-(N-4-Methylpiperazinyl)pentyl-Sar]-3-cyclosporin |
| 77a | 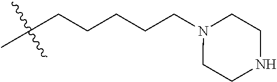 [SMILES: *CCCCCN1CCNCC1] | [(R)-5-(N-4-Piperazinyl)pentyl-Sar]-3-cyclosporin |

TABLE 2

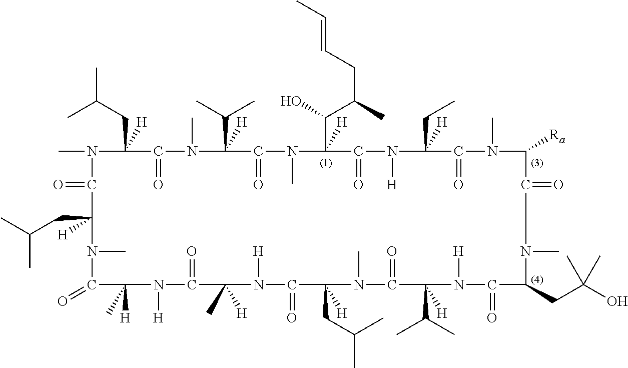

| Ex. | | |
|---|---|---|
| No. | Rₐ | Name |
| 78 | 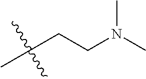 [SMILES: *CCN(C)C] | [(R)-2-(N,N-Dimethylamino)ethyl-Sar]-3-[(γ-hydroxy)- NMeLeu]-4-cyclosporin |
| 79 | 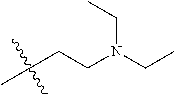 [SMILES: *CCN(CC)CC] | [(R)-2-(N,N-Diethylamino)ethyl-Sar]-3-[(γ-hydroxy)- NMeLeu]-4-cyclosporin |
| 80 | 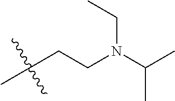 [SMILES: *CCN(CC)C(C)C] | [(R)-2-(N-Ethyl-N-isopropylamino)ethyl-Sar]-3-[(γ- hydroxy)-NMeLeu]-4-cyclosporin |
| 81 | 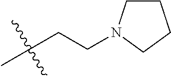 [SMILES: *CCN1CCCC1] | [(R)-2-(N-Pyrrolidinyl)ethyl-Sar]-3-[(γ-hydroxy)-NMeLeu]- 4-cyclosporin |
| 82 | 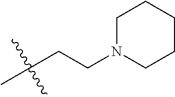 [SMILES: *CCN1CCCCC1] | [(R)-2-(N-Piperidinyl)ethyl-Sar]-3-[(γ-hydroxy)-NMeLeu]- 4-cyclosporin |
| 83 | 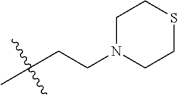 [SMILES: *CCN1CCSCC1] | [(R)-2-(N-Thiomorpholino)ethyl-Sar]-3-[(γ-hydroxy)- NMeLeu]-4-cyclosporin |
| 84 | 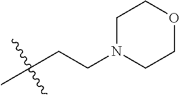 [SMILES: *CCN1CCOCC1] | [(R)-2-(N-Morpholino)ethyl-Sar]-3-[(γ-hydroxy)-NMeLeu]- 4-cyclosporin |
| 85 | 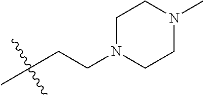 [SMILES: *CCN1CCN(C)CC1] | [(R)-2-(N-4-Methylpiperazinyl)ethyl-Sar]-3-[(γ-hydroxy)- NMeLeu]-4-cyclosporin |
| 85a | 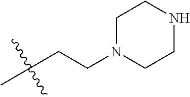 [SMILES: *CCN1CCNCC1] | [(R)-2-(N-4-Piperazinyl)ethyl-Sar]-3-[(γ-hydroxy)- NMeLeu]-4-cyclosporin |
| 86 | 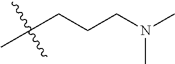 [SMILES: *CCCN(C)C] | [(R)-3-(N,N-Dimethylamino)propyl-Sar]-3-[(γ-hydroxy)- NmeLeu]-4-cyclosporin |
| 87 | 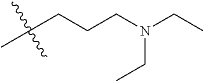 [SMILES: *CCCN(CC)CC] | [(R)-3-(N,N-Diethylamino)propyl-Sar]-3-[(γ-hydroxy)- NmeLeu]-4-cyclosporin |
| 88 | 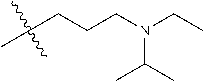 [SMILES: *CCCN(CC)C(C)C] | [(R)-3-(N-Ethyl-N-isopropylamino)propyl-Sar]-3-[(γ- hydroxy)-NmeLeu]-4-cyclosporin |
| 89 | 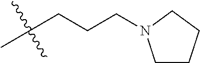 [SMILES: *CCCN1CCCC1] | [(R)-3-(N-Pyrrolidinyl)propyl-Sar]-3-[(γ-hydroxy)- NmeLeu]-4-cyclosporin |
| 90 | 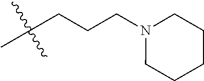 [SMILES: *CCCN1CCCCC1] | [(R)-3-(N-Piperidinyl)propyl-Sar]-3-[(γ-hydroxy)-NmeLeu]- 4-cyclosporin |
| 91 | 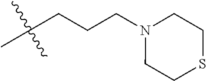 [SMILES: *CCCN1CCSCC1] | [(R)-3-(N-Thiomorpholino)propyl-Sar]-3-[(γ-hydroxy)- NmeLeu]-4-cyclosporin |
| 92 | 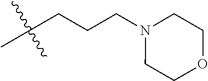 [SMILES: *CCCN1CCOCC1] | [(R)-3-(N-Morpholino)propyl-Sar]-3-[(γ-hydroxy)- NmeLeu]-4-cyclosporin |
| 93 | 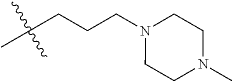 [SMILES: *CCCN1CCN(C)CC1] | [(R)-3-(N-4-Methylpiperazinyl)propyl-Sar]-3-[(γ-hydroxy)- NmeLeu]-4-cyclosporin |
| 93a | 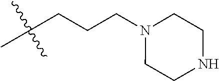 [SMILES: *CCCN1CCNCC1] | [(R)-3-(N-4-Piperazinyl)propyl-Sar]-3-[(γ-hydroxy)- NmeLeu]-4-cyclosporin |
| 94 | 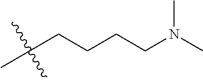 [SMILES: *CCCCN(C)C] | [(R)-4-(N,N-Dimethylamino)butyl-Sar]-3-[(γ-hydroxy)- NMeLeu]-4-cyclosporin |
| 95 | 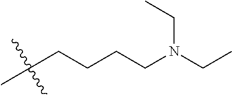 [SMILES: *CCCCN(CC)CC] | [(R)-4-(N,N-Diethylamino)butyl-Sar]-3-[(γ-hydroxy)- NmeLeu]-4-cyclosporin |
| 96 | 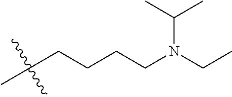 [SMILES: *CCCCN(CC)C(C)C] | [(R)-4-(N-Ethyl-N-isopropylamino)butyl-Sar]-3-[(γ- hydroxy)-NmeLeu]-4-cyclosporin |
| 97 | 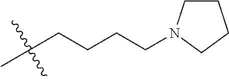 [SMILES: *CCCCN1CCCC1] | [(R)-4-(N-Pyrrolidinyl)butyl-Sar]-3-[(γ-hydroxy)-NmeLeu]- 4-cyclosporin |
| 98 | 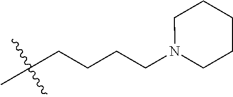 [SMILES: *CCCCN1CCCCC1] | [(R)-4-(N-Piperidinyl)butyl-Sar]-3-[(γ-hydroxy)-NmeLeu]- 4-cyclosporin |
| 99 | 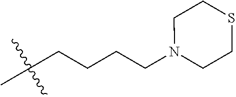 [SMILES: *CCCCN1CCSCC1] | [(R)-4-(N-Thiomorpholino)butyl-Sar]-3-[(γ-hydroxy)- NmeLeu]-4-cyclosporin |
| 100 | 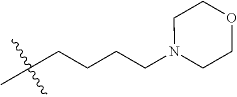 [SMILES: *CCCCN1CCOCC1] | [(R)-4-(N-Morpholino)butyl-Sar]-3-[(γ-hydroxy)-NmeLeu]- 4-cyclosporin |
| 101 | 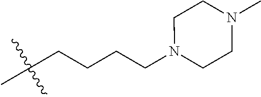 [SMILES: *CCCCN1CCN(C)CC1] | [(R)-4-(N-4-Methylpiperazinyl)butyl-Sar]-3-[(γ-hydroxy)- NmeLeu]-4-cyclosporin |
| 101a | 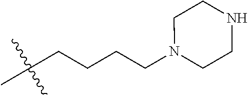 [SMILES: *CCCCN1CCNCC1] | [(R)-4-(N-4-Piperazinyl)butyl-Sar]-3-[(γ-hydroxy)- NmeLeu]-4-cyclosporin |
| 102 | 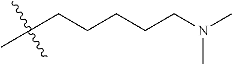 [SMILES: *CCCCCN(C)C] | [(R)-5-(N,N-Dimethylamino)pentyl-Sar]-3-[(γ-hydroxy)- NMeLeu]-4-cyclosporin |
| 103 | 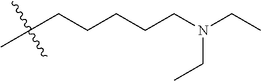 [SMILES: *CCCCCN(CC)CC] | [(R)-5-(N,N-Diethylamino)pentyl-Sar]-3-[(γ-hydroxy)- NMeLeu]-4-cyclosporin |
| 104 | 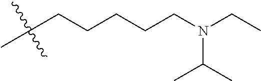 [SMILES: *CCCCCN(CC)C(C)C] | [(R)-5-(N-Ethyl-N-isopropylamino)butyl-Sar]-3-[(γ- hydroxy)-NMeLeu]-4-cyclosporin |
| 105 | 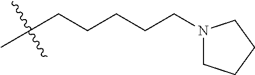 [SMILES: *CCCCCN1CCCC1] | [(R)-5-(N-Pyrrolidinyl)pentyl-Sar]-3-[(γ-hydroxy)- NMeLeu]-4-cyclosporin |
| 106 | 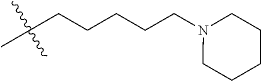 [SMILES: *CCCCCN1CCCCC1] | [(R)-5-(N-Piperidinyl)pentyl-Sar]-3-[(γ-hydroxy)-NMeLeu]- 4-cyclosporin |
| 107 | 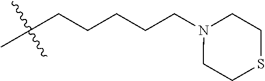 [SMILES: *CCCCCN1CCSCC1] | [(R)-5-(N-Thiomorpholino)pentyl-Sar]-3-[(γ-hydroxy)- NMeLeu]-4-cyclosporin |
| 108 | 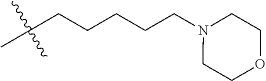 [SMILES: *CCCCCN1CCOCC1] | [(R)-5-(N-Morpholino)pentyl-Sar]-3-[(γ-hydroxy)- NMeLeu]-4-cyclosporin |
| 109 | 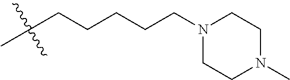 [SMILES: *CCCCCN1CCN(C)CC1] | [(R)-5-(N-4-Methylpiperazinyl)pentyl-Sar]-3-[(γ-hydroxy)- NMeLeu]-4-cyclosporin |
| 109a | 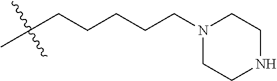 [SMILES: *CCCCCN1CCNCC1] | [(R)-5-(N-4-Piperazinyl)pentyl-Sar]-3-[(γ-hydroxy)- NmeLeu]-4-cyclosporin |

TABLE 3

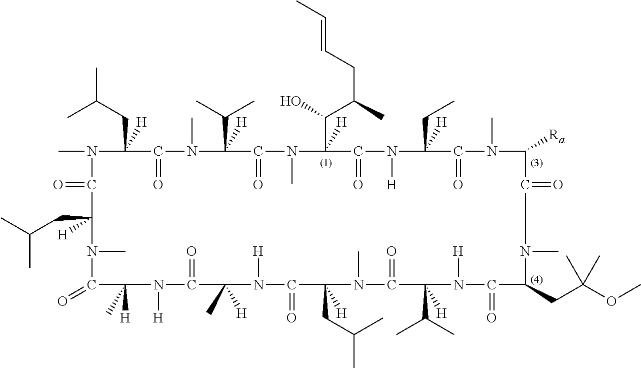

| Ex. | | |
|---|---|---|
| No. | Ra | Name |
| 110 | 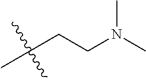 [SMILES: *CCN(C)C] | [(R)-2-(N,N-Dimethylamino)ethyl-Sar]-3-[(γ-methoxy)- NmeLeu]-4-cyclosporin |
| 111 | 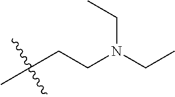 [SMILES: *CCN(CC)CC] | [(R)-2-(N,N-Diethylamino)ethyl-Sar]-3-[(γ-methoxy)- NmeLeu]-4-cyclosporin |
| 112 | 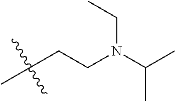 [SMILES: *CCN(CC)C(C)C] | [(R)-2-(N-Ethyl-N-isopropylamino)ethyl-Sar]-3-[(γ- methoxy)-NmeLeu]-4-cyclosporin |
| 113 | 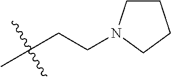 [SMILES: *CCN1CCCC1] | [(R)-2-(N-Pyrrolidinyl)ethyl-Sar]-3-[(γ-methoxy)-NmeLeu]- 4-cyclosporin |
| 114 | 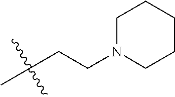 [SMILES: *CCN1CCCCC1] | [(R)-2-(N-Piperidinyl)ethyl-Sar]-3-[(γ-methoxy)-NmeLeu]- 4-cyclosporin |
| 115 | 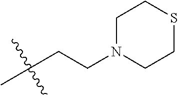 [SMILES: *CCN1CCSCC1] | [(R)-2-(N-Thiomorpholino)ethyl-Sar]-3-[(γ-methoxy)- NmeLeu]-4-cyclosporin |
| 116 | 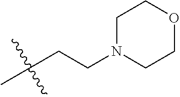 [SMILES: *CCN1CCOCC1] | [(R)-2-(N-Morpholino)ethyl-Sar]-3-[(γ-methoxy)-NmeLeu]- 4-cyclosporin |
| 117 | 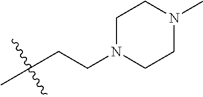 [SMILES: *CCN1CCN(C)CC1] | [(R)-2-(N-4-Methylpiperazinyl)ethyl-Sar]-3-[(γ-hydroxy)- NmeLeu]-4-cyclosporin |
| 117a | 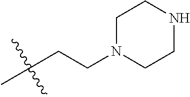 [SMILES: *CCN1CCNCC1] | [(R)-2-(N-4-Piperazinyl)ethyl-Sar]-3-[(γ-hydroxy)- NmeLeu]-4-cyclosporin |
| 118 | 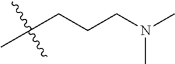 [SMILES: *CCCN(C)C] | [(R)-3-(N,N-Dimethylamino)propyl-Sar]-3-[(γ-methoxy)- NMeLeu]-4-cyclosporin |
| 119 | 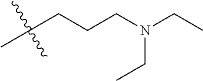 [SMILES: *CCCN(CC)CC] | [(R)-3-(N,N-Diethylamino)propyl-Sar]-3-[(γ-methoxy)- NmeLeu]-4-cyclosporin |
| 120 | 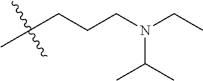 [SMILES: *CCCN(CC)C(C)C] | [(R)-3-(N-Ethyl-N-isopropylamino)propyl-Sar]-3-[(γ- methoxy)-NMeLeu]-4-cyclosporin |
| 121 | 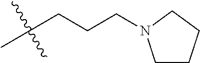 [SMILES: *CCCN1CCCC1] | [(R)-3-(N-Pyrrolidinyl)propyl-Sar]-3-[(γ-methoxy)- NMeLeu]-4-cyclosporin |
| 122 | 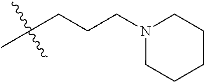 [SMILES: *CCCN1CCCCC1] | [(R)-3-(N-Piperidinyl)propyl-Sar]-3-[(γ-methoxy)- NMeLeu]-4-cyclosporin |
| 123 | 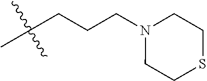 [SMILES: *CCCN1CCSCC1] | [(R)-3-(N-Thiomorpholino)propyl-Sar]-3-[(γ-methoxy)- NMeLeu]-4-cyclosporin |
| 124 | 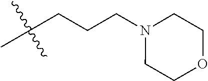 [SMILES: *CCCN1CCOCC1] | [(R)-3-(N-Morpholino)propyl-Sar]-3-[(γ-methoxy)- NMeLeu]-4-cyclosporin |
| 125 | 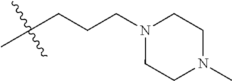 [SMILES: *CCCN1CCN(C)CC1] | [(R)-3-(N-4-Methylpiperazinyl)propyl-Sar]-3-[(γ-methoxy)- NMeLeu]-4-cyclosporin |
| 125a | 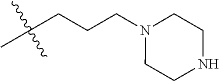 [SMILES: *CCCN1CCNCC1] | [(R)-3-(N-4-Piperazinyl)propyl-Sar]-3-[(γ-methoxy)- NMeLeu]-4-cyclosporin |
| 126 | 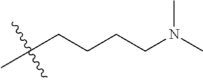 [SMILES: *CCCCN(C)C] | [(R)-4-(N,N-Dimethylamino)butyl-Sar]-3-[(γ-methoxy)- NmeLeu]-4-cyclosporin |
| 127 | 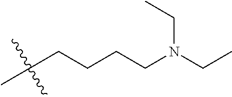 [SMILES: *CCCCN(CC)CC] | [(R)-4-(N,N-Diethylamino)butyl-Sar]-3-[(γ-methoxy)- NmeLeu]-4-cyclosporin |
| 128 | 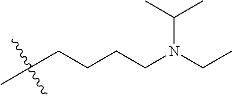 [SMILES: *CCCCN(CC)C(C)C] | [(R)-4-(N-Ethyl-N-isopropylamino)butyl-Sar]-3-[(γ- methoxy)-NMeLeu]-4-cyclosporin |
| 129 | 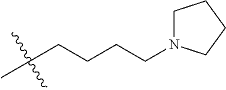 [SMILES: *CCCCN1CCCC1] | [(R)-4-(N-Pyrrolidinyl)butyl-Sar]-3-[(γ-methoxy)-NMeLeu]- 4-cyclosporin |
| 130 | 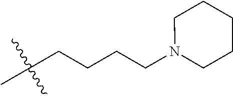 [SMILES: *CCCCN1CCCCC1] | [(R)-4-(N-Piperidinyl)butyl-Sar]-3-[(γ-methoxy)-NMeLeu] 4-cyclosporin |
| 131 | 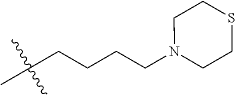 [SMILES: *CCCCN1CCSCC1] | [(R)-4-(N-Thiomorpholino)butyl-Sar]-3-[(γ-methoxy)- NMeLeu]-4-cyclosporin |
| 132 | 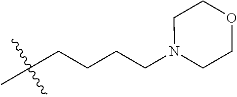 [SMILES: *CCCCN1CCOCC1] | [(R)-4-(N-Morpholino)butyl-Sar]-3-[(γ-methoxy)-NMeLeu]- 4-cyclosporin |
| 133 | 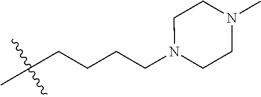 [SMILES: *CCCCN1CCN(C)CC1] | [(R)-4-(N-4-Methylpiperazinyl)butyl-Sar]-3-[(γ-metoxy)- NMeLeu]-4-cyclosporin |
| 133a | 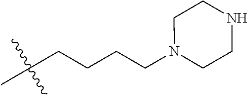 [SMILES: *CCCCN1CCNCC1] | [(R)-4-(N-4-Piperazinyl)butyl-Sar]-3-[(γ-metoxy)-NMeLeu]- 4-cyclosporin |
| 134 | 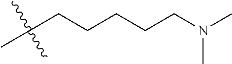 [SMILES: *CCCCCN(C)C] | [(R)-5-(N,N-Dimethylamino)pentyl-Sar]-3-[(γ-methoxy)- NMeLeu]-4-cyclosporin |
| 135 | 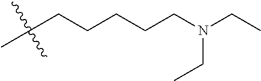 [SMILES: *CCCCCN(CC)CC] | [(R)-5-(N,N-Diethylamino)pentyl-Sar]-3-[(γ-methoxy)- NMeLeu]-4-cyclosporin |
| 136 | 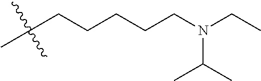 [SMILES: *CCCCCN(CC)C(C)C] | [(R)-5-(N-Ethyl-N-isopropylamino)pentyl-Sar]-3-[(γ- methoxy)-NMeLeu]-4-cyclosporin |
| 137 | 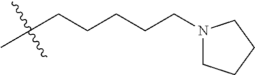 [SMILES: *CCCCCN1CCCC1] | [(R)-5-(N-Pyrrolidinyl)pentyl-Sar]-3-[(γ-methoxy)- NMeLeu]-4-cyclosporin |
| 138 | 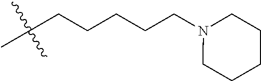 [SMILES: *CCCCCN1CCCCC1] | [(R)-5-(N-Piperidinyl)pentyl-Sar]-3-[(γ-methoxy)-NMeLeu]- 4-cyclosporin |
| 139 | 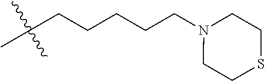 [SMILES: *CCCCCN1CCSCC1] | [(R)-5-(N-Thiomorpholino)pentyl-Sar]-3-[(γ-methoxy)- NMeLeu]-4-cyclosporin |
| 140 | 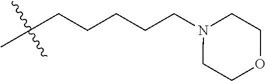 [SMILES: *CCCCCN1CCOCC1] | [(R)-5-(N-Morpholino)pentyl-Sar]-3-[(γ-methoxy)- NMeLeu]-4-cyclosporin |
| 141 | 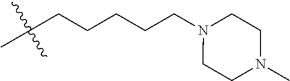 [SMILES: *CCCCCN1CCN(C)CC1] | [(R)-5-(N-4-Methylpiperazinyl)pentyl-Sar]-3-[(γ-methoxy)- NMeLeu]-4-cyclosporin |
| 141a | 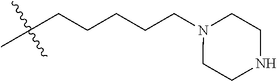 [SMILES: *CCCCCN1CCNCC1] | [(R)-5-(N-4-Piperazinyl)pentyl-Sar]-3-[(γ-methoxy)- NMeLeu]-4-cyclosporin |

TABLE 4

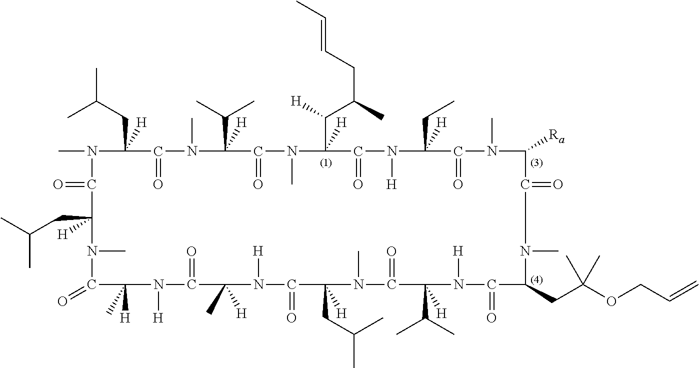

| Ex. | | |
|---|---|---|
| No. | Rₐ | Name |
| 142 | 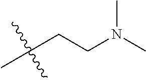 [SMILES: *CCN(C)C] | [(R)-2-(N,N-Dimethylamino)ethyl-Sar]-3-[(γ-allyloxy)- NMeLeu]-4-cyclosporin |
| 143 | 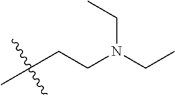 [SMILES: *CCN(CC)CC] | [(R)-2-(N,N-Diethylamino)ethyl-Sar]-3-[(γ-allyloxy)- NMeLeu]-4-cyclosporin |
| 144 | 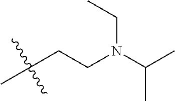 [SMILES: *CCN(CC)C(C)C] | [(R)-2-(N-Ethyl-N-isopropylamino)ethyl-Sar]-3-[(γ- allyloxy)-NMeLeu]-4-cyclosporin |
| 145 | 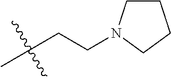 [SMILES: *CCN1CCCC1] | [(R)-2-(N-Pyrrolidinyl)ethyl-Sar]-3-[(γ-allyloxy)-NMeLeu]- 4-cyclosporin |
| 146 | 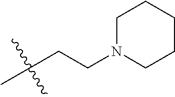 [SMILES: *CCN1CCCCC1] | [(R)-2-(N-Piperidinyl)ethyl-Sar]-3-[(γ-allyloxy)-NMeLeu]- 4-cyclosporin |
| 147 | 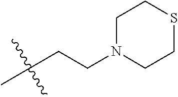 [SMILES: *CCN1CCSCC1] | [(R)-2-(N-Thiomorpholino)ethyl-Sar]-3-[(γ-allyloxy)- NMeLeu]-4-cyclosporin |
| 148 | 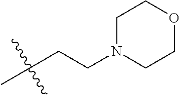 [SMILES: *CCN1CCOCC1] | [(R)-2-(N-Morpholino)ethyl-Sar]-3-[(γ-allyloxy)-NMeLeu]- 4-cyclosporin |
| 149 | 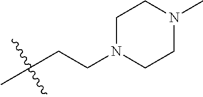 [SMILES: *CCN1CCN(C)CC1] | [(R)-2-(N-4-Methylpiperazinyl)ethyl-Sar]-3-[(γ-allyloxy)- NMeLeu]-4-cyclosporin |
| 149a | 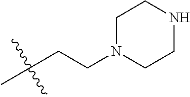 [SMILES: *CCN1CCNCC1] | [(R)-2-(N-4-Piperazinyl)ethyl-Sar]-3-[(γ-allyloxy)- NMeLeu]-4-cyclosporin |
| 150 | 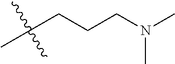 [SMILES: *CCCN(C)C] | [(R)-3-(N,N-Dimethylamino)propyl-Sar]-3-[(γ-allyloxy)- NMeLeu]-4-cyclosporin |
| 151 | 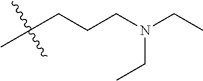 [SMILES: *CCCN(CC)CC] | [(R)-3-(N,N-Diethylamino)propyl-Sar]-3-[(γ-allyloxy)- NMeLeu]-4-cyclosporin |
| 152 | 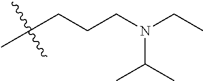 [SMILES: *CCCN(CC)C(C)C] | [(R)-3-(N-Ethyl-N-isopropylamino)propyl-Sar]-3-[(γ- allyloxy)-NMeLeu]-4-cyclosporin |
| 153 | 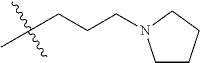 [SMILES: *CCCN1CCCC1] | [(R)-3-(N-Pyrrolidinyl)propyl-Sar]-3-[(γ-allyloxy)- NMeLeu]-4-cyclosporin |
| 154 | 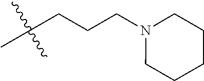 [SMILES: *CCCN1CCCCC1] | [(R)-3-(N-Piperidinyl)propyl-Sar]-3-[(γ-allyloxy)-NMeLeu]- 4-cyclosporin |
| 155 | 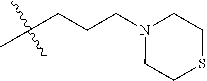 [SMILES: *CCCN1CCSCC1] | [(R)-3-(N-Thiomorpholino)propyl-Sar]-3-[(γ-allyloxy)- NMeLeu]-4-cyclosporin |
| 156 | 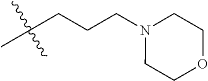 [SMILES: *CCCN1CCOCC1] | [(R)-3-(N-Morpholino)propyl-Sar]-3-[(γ-allyloxy)- NMeLeu]-4-cyclosporin |
| 157 | 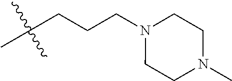 [SMILES: *CCCN1CCN(C)CC1] | [(R)-3-(N-4-Methylpiperazinyl)propyl-Sar]-3-[(γ-allyloxy)- NMeLeu]-4-cyclosporin |
| 157a | 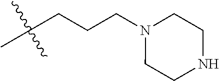 [SMILES: *CCCN1CCNCC1] | [(R)-3-(N-4-Piperazinyl)propyl-Sar]-3-[(γ-allyloxy)- NMeLeu]-4-cyclosporin |
| 158 | 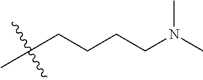 [SMILES: *CCCCN(C)C] | [(R)-4-(N,N-Dimethylamino)butyl-Sar]-3-[(γ-allyloxy)- NMeLeu]-4-cyclosporin |
| 159 | 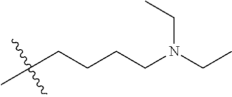 [SMILES: *CCCCN(CC)CC] | [(R)-4-(N,N-Diethylamino)butyl-Sar]-3-[(γ-allyloxy)- NMeLeu]-4-cyclosporin |
| 160 | 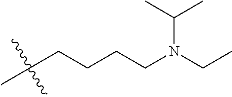 [SMILES: *CCCCN(CC)C(C)C] | [(R)-4-(N-Ethyl-N-isopropylamino)butyl-Sar]-3-[(γ- allyloxy)-NMeLeu]-4-cyclosporin |
| 161 | 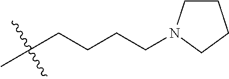 [SMILES: *CCCCN1CCCC1] | [(R)-4-(N-Pyrrolidinyl)butyl-Sar]-3-[(γ-allyloxy)-NMeLeu]- 4-cyclosporin |
| 162 | 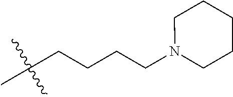 [SMILES: *CCCCN1CCCCC1] | [(R)-4-(N-Piperidinyl)butyl-Sar]-3-[(γ-allyloxy)-NMeLeu]- 4-cyclosporin |
| 163 | 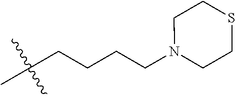 [SMILES: *CCCCN1CCSCC1] | [(R)-4-(N-Thiomorpholino)butyl-Sar]-3-[(γ-allyloxy)- NMeLeu]-4-cyclosporin |
| 164 | 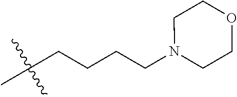 [SMILES: *CCCCN1CCOCC1] | [(R)-4-(N-Morpholino)butyl-Sar]-3-[(γ-allyloxy)-NMeLeu]- 4-cyclosporin |
| 165 | 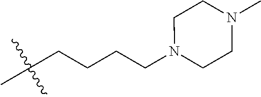 [SMILES: *CCCCN1CCN(C)CC1] | [(R)-4-(N-4-Methylpiperazinyl)butyl-Sar]-3-[(γ-allyloxy)- NMeLeu]-4-cyclosporin |
| 165a | 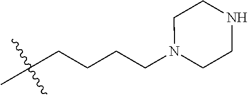 [SMILES: *CCCCN1CCNCC1] | [(R)-4-(N-4-Piperazinyl)butyl-Sar]-3-[(γ-allyloxy)- NMeLeu]-4-cyclosporin |
| 166 | 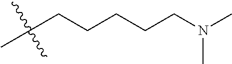 [SMILES: *CCCCCN(C)C] | [(R)-5-(N,N-Dimethylamino)pentyl-Sar]-3-[(γ-allyloxy)- NMeLeu]-4-cyclosporin |
| 167 | 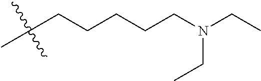 [SMILES: *CCCCCN(CC)CC] | [(R)-5-(N,N-Diethylamino)pentyl-Sar]-3-[(γ-allyloxy)- NMeLeu]-4-cyclosporin |
| 168 | 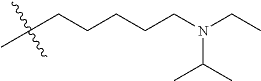 [SMILES: *CCCCCN(CC)C(C)C] | [(R)-5-(N-Ethyl-N-isopropylamino)pentyl-Sar]-3-[(γ- allyloxy)-NMeLeu]-4-cyclosporin |
| 169 | 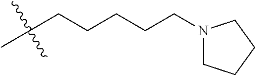 [SMILES: *CCCCCN1CCCC1] | [(R)-5-(N-Pyrrolidinyl)pentyl-Sar]-3-[(γ-allyloxy)- NMeLeu]-4-cyclosporin |
| 170 | 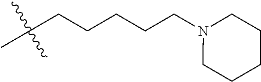 [SMILES: *CCCCCN1CCCCC1] | [(R)-5-(N-Piperidinyl)pentyl-Sar]-3-[(γ-allyloxy)-NMeLeu]- 4-cyclosporin |
| 171 | 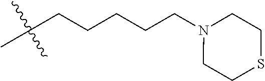 [SMILES: *CCCCCN1CCSCC1] | [(R)-5-(N-Thiomorpholino)pentyl-Sar]-3-[(γ-allyloxy)- NMeLeu]-4-cyclosporin |
| 172 | 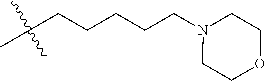 [SMILES: *CCCCCN1CCOCC1] | [(R)-5-(N-Morpholino)pentyl-Sar]-3-[(γ-allyloxy)- NMeLeu]-4-cyclosporin |
| 173 | 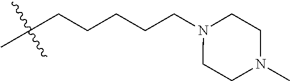 [SMILES: *CCCCCN1CCN(C)CC1] | [(R)-5-(N-4-Methylpiperazinyl)pentyl-Sar]-3-[(γ-allyloxy)- NMeLeu]-4-cyclosporin |
| 173a | 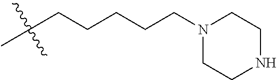 [SMILES: *CCCCCN1CCNCC1] | [(R)-5-(N-4-Piperazinyl)pentyl-Sar]-3-[(γ-allyloxy)- NMeLeu]-4-cyclosporin |

TABLE 5

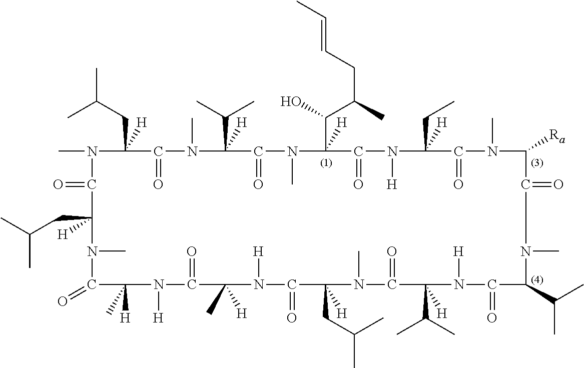

| Ex. | | |
|---|---|---|
| No. | Rₐ | Name |
| 174 | 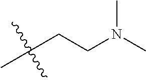 [SMILES: *CCN(C)C] | [(R)-2-(N,N-Dimethylamino)ethyl-Sar]-3-[NMeVal]-4- cyclosporin |
| 175 | 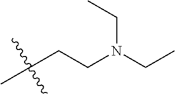 [SMILES: *CCN(CC)CC] | [(R)-2-(N,N-Diethylamino)ethyl-Sar]-3-[NMeVal]-4- cyclosporin |
| 176 | 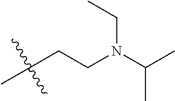 [SMILES: *CCN(CC)C(C)C] | [(R)-2-(N-Ethyl-N-isopropylamino)ethyl-Sar]-3-[NMeVal]- 4-cyclosporin |
| 177 | 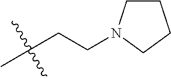 [SMILES: *CCN1CCCC1] | [(R)-2-(N-Pyrrolidinyl)ethyl-Sar]-3-[NMeVal]-4-cyclosporin |
| 178 | 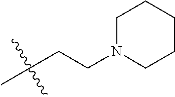 [SMILES: *CCN1CCCCC1] | [(R)-2-(N-Piperidinyl)ethyl-Sar]-3-[NMeVal]-4-cyclosporin |
| 179 | 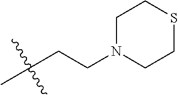 [SMILES: *CCN1CCSCC1] | [(R)-2-(N-Thiomorpholino)ethyl-Sar]-3-[NMeVal]-4- cyclosporin |
| 180 | 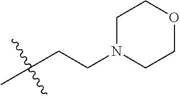 [SMILES: *CCN1CCOCC1] | [(R)-2-(N-Morpholino)ethyl-Sar]-3-[NMeVal]-4-cyclosporin |
| 181 | 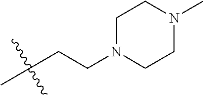 [SMILES: *CCN1CCN(C)CC1] | [(R)-2-(N-4-Methylpiperazinyl)ethyl-Sar]-3-[NMeVal]-4- cyclosporin |
| 181a | 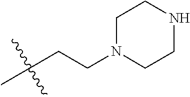 [SMILES: *CCN1CCNCC1] | [(R)-2-(N-4-Piperazinyl)ethyl-Sar]-3-[NMeVal]-4- cyclosporin |
| 182 | 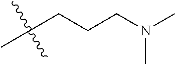 [SMILES: *CCCN(C)C] | [(R)-3-(N,N-Dimethylamino)propyl-Sar]-3-[NMeVal]-4- cyclosporin |
| 183 | 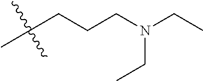 [SMILES: *CCCN(CC)CC] | [(R)-3-(N,N-Diethylamino)propyl-Sar]-3-[NMeVal]-4- cyclosporin |
| 184 | 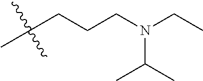 [SMILES: *CCCN(CC)C(C)C] | [(R)-3-(N-Ethyl-N-isopropylamino)propyl-Sar]-3- [NMeVal]-4-cyclosporin |
| 185 | 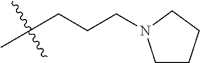 [SMILES: *CCCN1CCCC1] | [(R)-3-(N-Pyrrolidinyl)propyl-Sar]-3-[NMeVal]-4- cyclosporin |
| 186 | 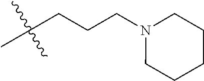 [SMILES: *CCCN1CCCCC1] | [(R)-3-(N-Piperidinyl)propyl-Sar]-3-[NMeVal]-4- cyclosporin |
| 187 | 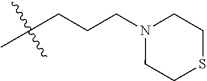 [SMILES: *CCCN1CCSCC1] | [(R)-3-(N-Thiomorpholino)propyl-Sar]-3-[NMeVal]-4- cyclosporin |
| 188 | 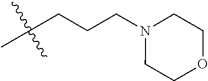 [SMILES: *CCCN1CCOCC1] | [(R)-3-(N-Morpholino)propyl-Sar]-3-[NMeVal]-4- cyclosporin |
| 189 | 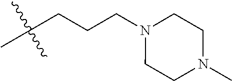 [SMILES: *CCCN1CCN(C)CC1] | [(R)-3-(N-4-Methylpiperazinyl)propyl-Sar]-3-[NMeVal]-4- cyclosporin |
| 189a | 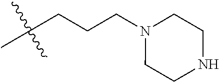 [SMILES: *CCCN1CCNCC1] | [(R)-3-(N-4-Piperazinyl)propyl-Sar]-3-[NMeVal]-4- cyclosporin |
| 190 | 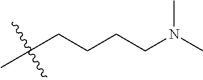 [SMILES: *CCCCN(C)C] | [(R)-4-(N,N-Dimethylamino)butyl-Sar]-3-[NMeVal]-4- cyclosporin |
| 191 | 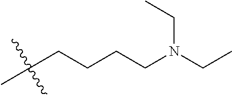 [SMILES: *CCCCN(CC)CC] | [(R)-4-(N,N-Diethylamino)butyl-Sar]-3-[NMeVal]-4- cyclosporin |
| 192 | 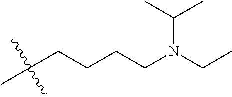 [SMILES: *CCCCN(CC)C(C)C] | [(R)-4-(N-Ethyl-N-isopropylamino)butyl-Sar]-3-[NMeVal]- 4-cyclosporin |
| 193 | 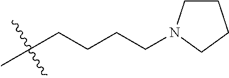 [SMILES: *CCCCN1CCCC1] | [(R)-4-(N-Pyrrolidinyl)butyl-Sar]-3-[NMeVal]-4- cyclosporin |
| 194 | 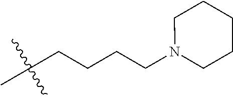 [SMILES: *CCCCN1CCCCC1] | [(R)-4-(N-Piperidinyl)butyl-Sar]-3-[NMeVal]-4-cyclosporin |
| 195 | 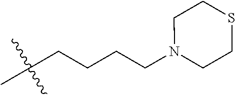 [SMILES: *CCCCN1CCSCC1] | [(R)-4-(N-Thiomorpholino)butyl-Sar]-3-[NMeVal]-4- cyclosporin |
| 196 | 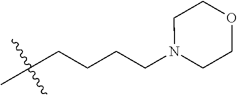 [SMILES: *CCCCN1CCOCC1] | [(R)-4-(N-Morpholino)butyl-Sar]-3-[NMeVal]-4-cyclosporin |
| 197 | 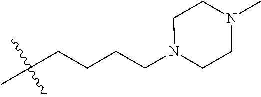 [SMILES: *CCCCN1CCN(C)CC1] | [(R)-4-(N-4-Methylpiperazinyl)butyl-Sar]-3-[NMeVal]-4- cyclosporin |
| 197a | 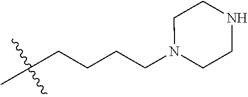 [SMILES: *CCCCN1CCNCC1] | [(R)-4-(N-4-Piperazinyl)butyl-Sar]-3-[NMeVal]-4- cyclosporin |
| 198 | 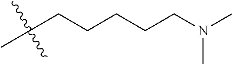 [SMILES: *CCCCCN(C)C] | [(R)-5-(N,N-Dimethylamino)pentyl-Sar]-3-[NMeVal]-4- cyclosporin |
| 199 | 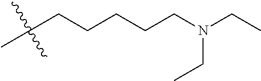 [SMILES: *CCCCCN(CC)CC] | [(R)-5-(N,N-Diethylamino)pentyl-Sar]-3-[NMeVal]-4- cyclosporin |
| 200 | 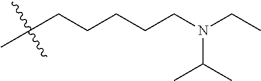 [SMILES: *CCCCCN(CC)C(C)C] | [(R)-5-(N-Ethyl-N-isopropylamino)pentyl-Sar]-3-[NMeVal]- 4-cyclosporin |
| 201 | 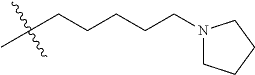 [SMILES: *CCCCCN1CCCC1] | [(R)-5-(N-Pyrrolidinyl)pentyl-Sar]-3-[NMeVal]-4- cyclosporin |
| 202 | 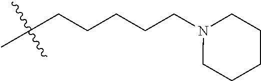 [SMILES: *CCCCCN1CCCCC1] | [(R)-5-(N-Piperidinyl)pentyl-Sar]-3-[NMeVal]-4- cyclosporin |
| 203 | 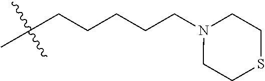 [SMILES: *CCCCCN1CCSCC1] | [(R)-5-(N-Thiomorpholino)pentyl-Sar]-3-[NMeVal]-4- cyclosporin |
| 204 | 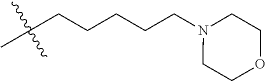 [SMILES: *CCCCCN1CCOCC1] | [(R)-5-(N-Morpholino)pentyl-Sar]-3-[NMeVal]-4- cyclosporin |
| 205 | 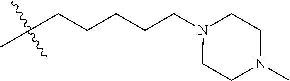 [SMILES: *CCCCCN1CCN(C)CC1] | [(R)-5-(N-4-Methylpiperazinyl)pentyl-Sar]-3-[NMeVal]-4- cyclosporin |
| 205a | 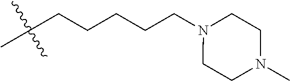 [SMILES: *CCCCCN1CCN(C)CC1] | [(R)-5-(N-4-Piperazinyl)pentyl-Sar]-3-[NMeVal]-4- cyclosporin |

TABLE 6

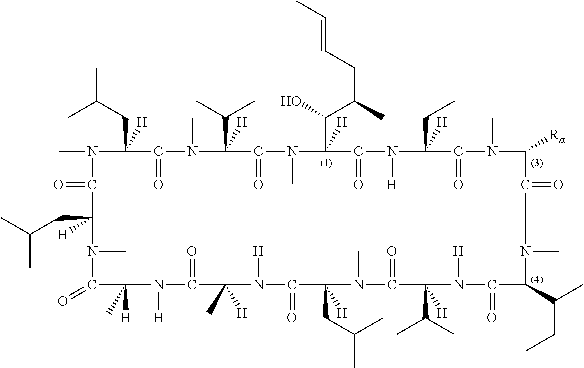

| Ex. | | |
|---|---|---|
| No. | Rₐ | Name |
| 206 | 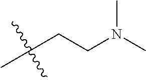 [SMILES: *CCN(C)C] | [(R)-2-(N,N-Dimethylamino)ethyl-Sar]-3-[NMeIle]-4- cyclosporin |
| 207 | 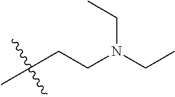 [SMILES: *CCN(CC)CC] | [(R)-2-(N,N-Diethylamino)ethyl-Sar]-3-[NMeIle]-4- cyclosporin |
| 208 | 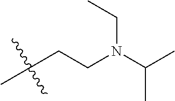 [SMILES: *CCN(CC)C(C)C] | [(R)-2-(N-Ethyl-N-isopropylamino)ethyl-Sar]-3-[NMeIle]-4- cyclosporin |
| 209 | 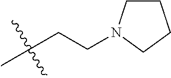 [SMILES: *CCN1CCCC1] | [(R)-2-(N-Pyrrolidinyl)ethyl-Sar]-3-[NMeIle]-4-cyclosporin |
| 210 | 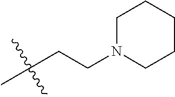 [SMILES: *CCN1CCCCC1] | [(R)-2-(N-Piperidinyl)ethyl-Sar]-3-[NMeIle]-4-cyclosporin |
| 211 | 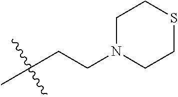 [SMILES: *CCN1CCSCC1] | [(R)-2-(N-Thiomorpholino)ethyl-Sar]-3-[NMeIle]-4- cyclosporin |
| 212 | 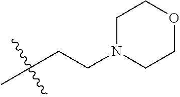 [SMILES: *CCN1CCOCC1] | [(R)-2-(N-Morpholino)ethyl-Sar]-3-[NMeIle]-4-cyclosporin |
| 213 | 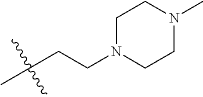 [SMILES: *CCN1CCN(C)CC1] | [(R)-2-(N-4-Methylpiperazinyl)ethyl-Sar]-3-[NMeIle]-4- cyclosporin |
| 213a | 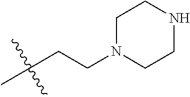 [SMILES: *CCN1CCNCC1] | [(R)-2-(N-4-Piperazinyl)ethyl-Sar]-3-[NMeIle]-4- cyclosporin |
| 214 | 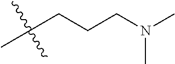 [SMILES: *CCCN(C)C] | [(R)-3-(N,N-Dimethylamino)propyl-Sar]-3-[NMeIle]-4- cyclosporin |
| 215 | 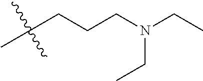 [SMILES: *CCCN(CC)CC] | [(R)-3-(N,N-Diethylamino)propyl-Sar]-3-[NMeIle]-4- cyclosporin |
| 216 | 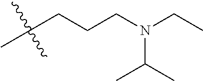 [SMILES: *CCCN(CC)C(C)C] | [(R)-3-(N-Ethyl-N-isopropylamino)propyl-Sar]-3-[NMeIle]- 4-cyclosporin |
| 217 | 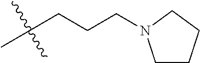 [SMILES: *CCCN1CCCC1] | [(R)-3-(N-Pyrrolidinyl)propyl-Sar]-3-[NMeIle]-4- cyclosporin |
| 218 | 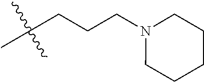 [SMILES: *CCCN1CCCCC1] | [(R)-3-(N-Piperidinyl)propyl-Sar]-3-[NMeIle]-4-cyclosporin |
| 219 | 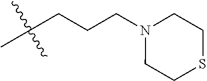 [SMILES: *CCCN1CCSCC1] | [(R)-3-(N-Thiomorpholino)propyl-Sar]-3-[NMeIle]-4- cyclosporin |
| 220 | 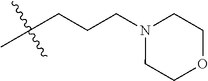 [SMILES: *CCCN1CCOCC1] | [(R)-3-(N-Morpholino)propyl-Sar]-3-[NMeIle]-4- cyclosporin |
| 221 | 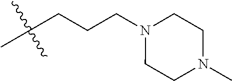 [SMILES: *CCCN1CCN(C)CC1] | [(R)-3-(N-4-Methylpiperazinyl)propyl-Sar]-3-[NMeIle]-4- cyclosporin |
| 221a | 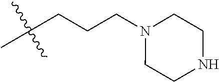 [SMILES: *CCCN1CCNCC1] | [(R)-3-(N-4-Piperazinyl)propyl-Sar]-3-[NMeIle]-4- cyclosporin |
| 222 | 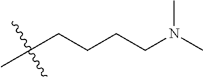 [SMILES: *CCCCN(C)C] | [(R)-4-(N,N-Dimethylamino)butyl-Sar]-3-[NMeIle]-4- cyclosporin |
| 223 | 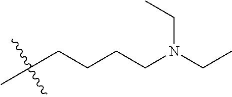 [SMILES: *CCCCN(CC)CC] | [(R)-4-(N,N-Diethylamino)butyl-Sar]-3-[NMeIle]-4- cyclosporin |
| 224 | 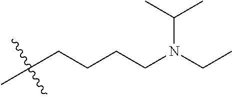 [SMILES: *CCCCN(CC)C(C)C] | [(R)-4-(N-Ethyl-N-isopropylamino)butyl-Sar]-3-[N-MeIle]- 4-cyclosporin |
| 225 | 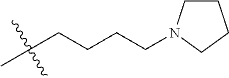 [SMILES: *CCCCN1CCCC1] | [(R)-4-(N-Pyrrolidinyl)butyl-Sar]-3-[NMeIle]-4-cyclosporin |
| 226 | 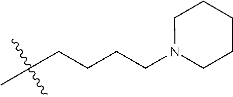 [SMILES: *CCCCN1CCCCC1] | [(R)-4-(N-Piperidinyl)butyl-Sar]-3-[NMeIle]-4-cyclosporin |
| 227 | 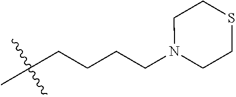 [SMILES: *CCCCN1CCSCC1] | [(R)-4-(N-Thiomorpholino)butyl-Sar]-3-[NMeIle]-4- cyclosporin |
| 228 | 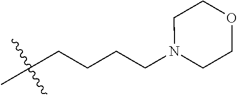 [SMILES: *CCCCN1CCOCC1] | [(R)-4-(N-Morpholino)butyl-Sar]-3-[NMeIle]-4-cyclosporin |
| 229 | 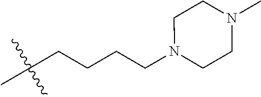 [SMILES: *CCCCN1CCN(C)CC1] | [(R)-4-(N-4-Methylpiperazinyl)butyl-Sar]-3-[NMeIle]-4- cyclosporin |
| 229a | 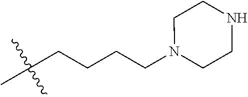 [SMILES: *CCCCN1CCNCC1] | [(R)-4-(N-4-Piperazinyl)butyl-Sar]-3-[NMeIle]-4- cyclosporin |
| 230 | 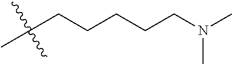 [SMILES: *CCCCCN(C)C] | [(R)-5-(N,N-Dimethylamino)pentyl-Sar]-3-[NMeIle]-4- cyclosporin |
| 231 | 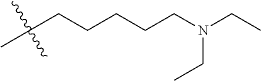 [SMILES: *CCCCCN(CC)CC] | [(R)-5-(N,N-Diethylamino)pentyl-Sar]-3-[NMeIle]-4- cyclosporin |
| 232 | 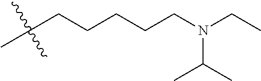 [SMILES: *CCCCCN(CC)C(C)C] | [(R)-5-(N-Ethyl-N-isopropylamino)pentyl-Sar]-3-[NMeIle]- 4-cyclosporin |
| 233 | 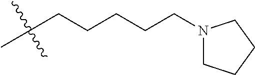 [SMILES: *CCCCCN1CCCC1] | [(R)-5-(N-Pyrrolidinyl)pentyl-Sar]-3-[NMeIle]-4- cyclosporin |
| 234 | 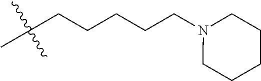 [SMILES: *CCCCCN1CCCCC1] | [(R)-5-(N-Piperidinyl)pentyl-Sar]-3-[NMeIle]-4-cyclosporin |
| 235 | 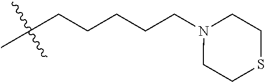 [SMILES: *CCCCCN1CCSCC1] | [(R)-5-(N-Thiomorpholino)pentyl-Sar]-3-[NMelle]-4- cyclosporin |
| 236 | 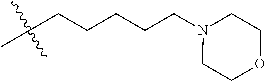 [SMILES: *CCCCCN1CCOCC1] | [(R)-5-(N-Morpholino)pentyl-Sar]-3-[NMeIle]-4- cyclosporin |
| 237 | 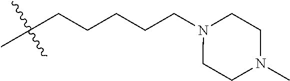 [SMILES: *CCCCCN1CCN(C)CC1] | [(R)-5-(N-4-Methylpiperazinyl)pentyl-Sar]-3-[NMeIle]-4- cyclosporin |
| 237a | 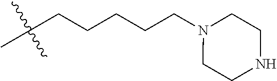 [SMILES: *CCCCCN1CCNCC1] | [(R)-5-(N-4-Piperazinyl)pentyl-Sar]-3-[NMeIle]-4- cyclosporin |

### Example 238

### Anti HCV Activity of Cyclosporin Derivatives

The anti-HCV activity of cyclosporine derivatives were evaluated in the HCV subgenomic replicon assay. The assay use the cell line ET (luc-ubi-neo/ET), which is a Huh7 human hepatoma cell line harboring an HCV replicon with a stable luciferase (Luc) reporter. HCV RNA replication was assessed by quantifying HCV replicon-derived luciferase activity. The antiviral activity of cyclosporine analogs were evaluated after drug treatment, the EC 50 and EC 90 were determined in subsequent assessments by using the luciferase end point (Krieger, N., et al., 2001, J. Virol. 75, 4614-4624; Pietschmann, T., et al., 2002, J. Virol. 76, 4008-4021; each of which is incorporated herein by reference).

The results of certain compounds are as follows:

| | Antiviral |
|---|---|
| | activity: |
| Compounds | IC₅₀ (μM) |
| [(R,S)-2-(N,N-Dimethylamino)ethyl-Sar]-3- | ** |
| cyclosporin | |
| [(R)-2-(N,N-Diethylamino)ethyl-Sar]-3- | *** |
| cyclosporin | |
| [(S)-2-(N,N-Diethylamino)ethyl-Sar]-3- | * |
| cyclosporin | |
| [(R)-2-(N,N-Dimethylamino)ethyl-Sar]-3- | *** |
| [(γ-Hydroxy)-NMeLeu]-4-cyclosporin | |
| [(S)-2-(N,N-Dimethylamino)ethyl-Sar]-3- | * |
| [(γ-Hydroxy)-NMeLeu]-4-cyclosporin | |
| [(R)-2-(N,N-Diethylamino)ethyl-Sar]-3-[(γ- | *** |
| Hydroxy)-NMeLeu]-4-cyclosporin | |
| [(S)-2-(N,N-Diethylamino)ethyl-Sar]-3-[(γ- | * |
| Hydroxy)-NMeLeu]-4-cyclosporin | |
| [(R)-2-(N,N-Dimethylamino)ethyl-Sar]-3- | *** |
| [(γ-methoxy)-NMeLeu]-4-cyclosporin | |
| [(R)-2-(N,N-Diethylamino)ethyl-Sar]-3-[(γ- | *** |
| methoxy)-NMeLeu]-4-cyclosporin | |
| [(R)-2-(N,N-Dimethylamino)ethyl-Sar]-3- | *** |
| [(γ-allyloxy)-NMeLeu]-4-cyclosporin | |
| [(R)-2-(N,N-Diethylamino)ethyl-Sar]-3-r(γ- | *** |
| allyloxy)-NMeLeu]-4-cyclosporin | |
| [(R)-2-(N,N-Dimethylamino)ethyl-Sar]-3- | *** |
| [NMeLeu]-4-cyclosporin | |
| [(R)-2-(N,N-Diethylamino)ethyl-Sar]-3- | *** |
| [NMeVal]-4-cyclosporin | |
| [(R)-2-(N,N-Dimethylamino)ethyl-Sar]-3- | *** |
| [NMeIle]-4-cyclosporin | |
| [(R)-2-(N,N-Diethylamino)ethyl-Sar]-3- | *** |
| [NMeIle]-4-cyclosporin | |
| [(R,S)-3-(N,N-Dimethylamino)propyl-Sar]- | ** |
| 3-cyclosporin | |
| [(R)-3-(N,N-Dimethylamino)propyl-Sar]-3- | *** |
| [(γ-hydroxy)-NMeLeu]-4-cyclosporin | |
| [(R)-3-(N,N-Dimethylamino)propyl-Sar]-3- | *** |
| [(γ-methoxy)-NMeLeu]-4-cyclosporin | |
| [(R)-3-(N,N-Dimethylamino)propyl-Sar]-3- | *** |
| [(γ-allyloxy)-NMeLeu]-4-cyclosporin | |
| [(R)-3-(N,N-Dimethylamino)propyl-Sar]-3- | *** |
| [NMeVal]-4-cyclosporin | |
| [(R)-3-(N,N-Dimethylamino)propyl-Sar]-3- | *** |
| [NMeIle]-4-cyclosporin | |

Antiviral activity:
***IC₅₀ < 0.25 μM;
**IC₅₀ < 1.5 μM;
*IC₅₀ < 5 μM

1. A compound of formula (I): 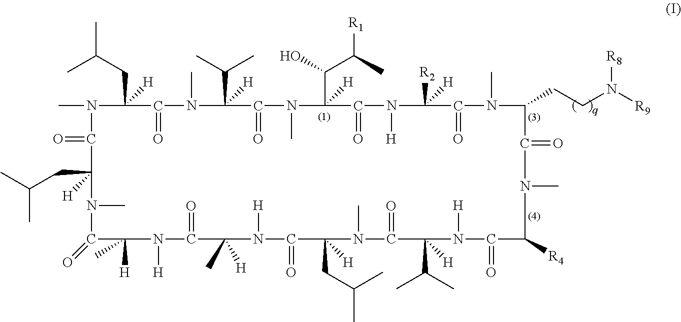
or pharmaceutically acceptable salt thereof, wherein the symbols have the following meanings and are, for each occurrence, independently selected:
R₁ is n-butyl, (E)-but-2-enyl;
R₂ is ethyl, 1-hydroxyethyl, isopropyl or n-propyl;
R₄ is 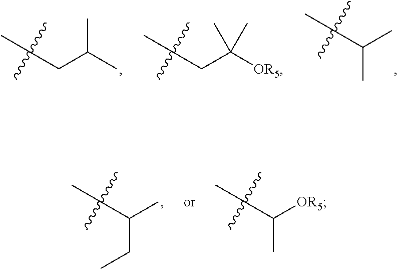
each R₅ is independently H, alkyl or substituted alkyl, alkenyl or substituted alkenyl, alkynyl or substituted alkynyl, cycloalkyl or substituted cycloalkyl, cycloalkenyl or substituted cycloalkenyl, or aryl or substituted aryl; and
each occurrence of R₈ and R₉ is independently H, alkyl or substituted alkyl, alkenyl or substituted alkenyl, alkynyl or substituted alkynyl, cycloalkyl or substituted cycloalkyl, phenyl or substituted phenyl, or R₈ and R₉, together with the nitrogen atom to which they are attached 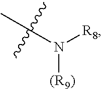
form a heterocycle or substituted heterocycle; and
q is an integer of 0, 1, 2, 3, 4, or 5.

2. The compound of claim 1, wherein:
R₁ is n-butyl or (E)-but-2-enyl;
R₂ is ethyl, 1-hydroxyethyl, isopropyl or n-propyl;
R₄ is 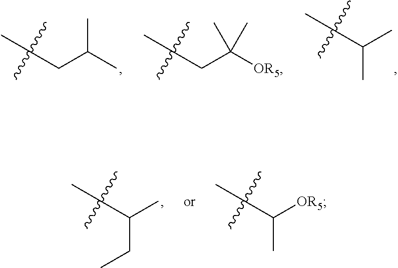
R₅ is:
H;
(C₁-C₆)alkyl, optionally substituted by one or more groups R₇ which may be the same or different;
(C₂-C₆)alkenyl, optionally substituted by one or more groups which may be the same or different selected from hydroxy, (C₁-C₆)alkyl, aryl (e.g., phenyl), (CH₂)ₚOR_{A}, O(CH₂)ₘOH, O(CH₂)ₘO(CH₂)ₘOH, O(CH₂)ₘNR_{A}R_{B}, O(CH₂)ₘO(CH₂)ₘNR_{A}R_{B}, (CH₂)ₚNR_{A}R_{B}, (CH₂)ₚNR_{c}(CH₂)ₘNR_{A}R_{B}, (CH₂)ₚNR_{C}(CH₂)ₘNR_{C}(CH₂)ₘNR_{A}R_{B}, (CH₂)ₚC(═O)NR_{A}R_{B}, (CH₂)ₚC(═O)OR_{A};
(C₂-C₆)alkynyl, optionally substituted by one or one or more groups which may be the same or different selected from halogen, hydroxy, amino, monoalkylamino and dialkylamino;
(C₃-C₇)cycloalkyl, optionally substituted by one or more groups which may be the same or different selected from halogen, hydroxy, amino, monoalkylamino and dialkylamino;
phenyl or CH₂-phenyl, optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B}, (CH₂)ₚC(═O)OR_{A};
each occurrence of R₇ is independently halogen, hydroxy, aryl (e.g., phenyl), S(C₁-C₆)alkyl, SR_{A}, OR_{A}, O(CH₂)ₘOH, O(CH₂)ₘO(CH₂)ₘOH, O(CH₂)ₘO(C₁-C₆)alkyl, O(CH₂)ₘO(CH₂)ₘO(C₁-C₆)alkyl, C(═O)OR_{A}, C(═O)NR_{A}R_{B}, NR_{A}R_{B}, O(CH₂)ₘNR_{A}R_{B}, O(CH₂)ₘ0(CH₂)ₘNR_{A}R_{B}, NR_{C}(CH₂)ₘNR_{A}R_{B}, or NR_{c}(CH₂)ₘNR_{c}(CH₂)ₘNR_{A}R_{B}, wherein said aryl or phenyl is optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B} and (CH₂)ₚC(═O)OR_{A};
each occurrence of R₈ and R₉ is independently H, alkyl, alkenyl, alkynyl, cycloalkyl, or phenyl, in which said alkyl, alkenyl, alkynyl, cycloalkyl, and phenyl may be optionally substituted by one or more groups R₁₀ which may be the same or different, in which each occurrence of R₁₀ is independently halogen, hydroxy, O(C₁-C₄)alkyl, C(═O)(C₁-C₄)alkyl, C(═O)O(C₁-C₄)alkyl; or R₈ and R₉, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from (C₁-C₄)alkyl, phenyl and benzyl.
each occurrence of R_{A} and R_{B} is independently:
hydrogen;
(C₁-C₆)alkyl, optionally substituted by one or more groups R_{D} which may be the same or different;
(C₂-C₆)alkenyl or (C₂-C₆)alkynyl;
(C₃-C₇)cycloalkyl optionally substituted with (C₁-C₆)alkyl;
phenyl optionally substituted with from one to five groups which may be the same or different selected from halogen, —O(C₁-C₆)alkyl, —C(═O)O(C₁-C₆)alkyl, amino, alkylamino and dialkylamino;
or a heterocyclic ring which may be saturated or unsaturated containing five or six ring atoms and from one to three heteroatoms which may the same or different selected from nitrogen, sulfur and oxygen;
or R_{A} and R_{B}, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from the group consisting of alkyl, phenyl and benzyl;
each occurrence of R_{C} is independently hydrogen or (C₁-C₆)alkyl;
p is an integer of 0, 1, 2, 3, 4, or 5; and
m is an integer of 1, 2, 3, 4 or 5.

3. The compound of claim 1, wherein R₁ is n-butyl.

4. The compound of claim 1, wherein R₁ is (E)-but-2-enyl.

5. The compound of claim 1, wherein R₂ is ethyl.

6. The compound of claim 2, wherein each occurrence of R₈ and R₉ is independently hydrogen; (C₁-C₄)alkyl, optionally substituted by one or more groups R₁₀ which may be the same or different, in which each occurrence of R₁₀ is independently halogen, hydroxy, O(C₁-C₄)alkyl, C(═O)(C₁-C₄)alkyl, C(═O)O(C₁-C₄)alkyl; or R₈ and R₉, together with the nitrogen atom to which they are attached, form a saturated or unsaturated heterocyclic ring containing from three to seven ring atoms, which ring may optionally contain another heteroatom selected from the group consisting of nitrogen, oxygen and sulfur and may be optionally substituted by from one to four groups which may be the same or different selected from (C₁-C₄)alkyl, phenyl and benzyl.

7. The compound of claim 2, wherein R₈ and R₉, together with the nitrogen atom to which they are attached, form a saturated or unsaturated heterocyclic ring containing from three to seven ring atoms, which ring may optionally contain another heteroatom selected from nitrogen, oxygen and sulfur and may be optionally substituted by from one to four groups which may be the same or different selected from (C₁-C₄)alkyl, phenyl and benzyl.

8. The compound of claim 1, wherein 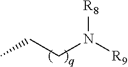
at (3)-position is 2-aminoethyl, 2-aminobutyl, 3-aminobutyl, 2-monoalkylaminoethyl, 2-monoalkylaminobutyl, 3-monoalkylaminobutyl, 2-dialkylaminoethyl, 2-dialkylaminobutyl, or 3-dialkylaminobutyl, wherein said alkyl is (C₁-C₄)alkyl.

9. The compound of claim 1, wherein 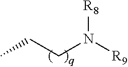
at (3)-position is dimethylaminoethyl, diethylaminoethyl, methylethylaminoethyl, methyl-iso-butylaminoethyl, ethyl-iso-butylaminoethyl, methyl-tert-butylaminoethyl, or ethyl-tert-butylaminoethyl.

10. The compound of claim 1, wherein 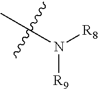
is: 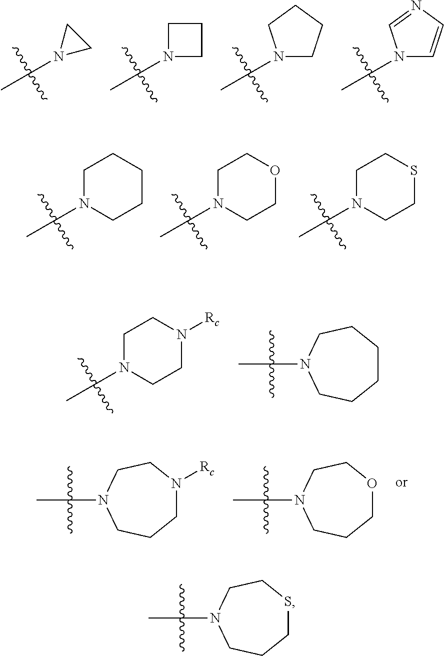
which R_{c} is H, Me, Et, n-Pr, i-Pr, n-Bu, i-Bu, t-Bu, CH₂CMe₃, Ph, or CH₂Ph.

11. The compound of claim 1, wherein R₅ is H, CH₂—S—(C₁-C₆)alky, CH₂—O—(C₁-C₆)alkyl, (C₂-C₆)alkenyl, benzyl, (C₂-C₆)OH, (C₁-C₆)-monoalkyl amine, (C₁-C₆)-dialkyl amine, or (C₁-C₆)-cyclic amine.

12. The compound of claim 1, wherein R₅ is H, methyl, ethyl, n-propyl, i-propyl, n-butyl, i-butyl, t-butyl, CH₂CMe₃, phenyl, CH₂-phenyl, 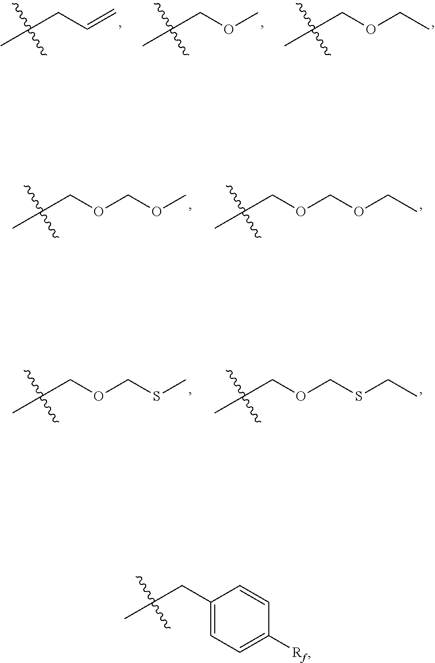
in which R_{f} is H, (C₁-C₄)alkyl or (C₁-C₄)alkoxy.

13. The compound of claim 1, wherein each occurrence of R₈ and R₉ is independently H, (C₁-C₆)alkyl, phenyl, CH₂-phenyl, (C₁-C₆)alkyl-OH, (C₁-C₆)-alkyl-O—(C₁-C₆)alkyl, (C₁-C₆)alkyl-O—(CH₂)ₘOH, (C₁-C₆)alkyl-O—(CH₂)ₘ—O—(C₁-C₆)alkyl, in which m is an integer of 1, 2, 3, 4 or 5.

14. The compound of claim 1, wherein R₈ and R₉, together with the nitrogen atom to which they are attached, form a heterocycle selected from 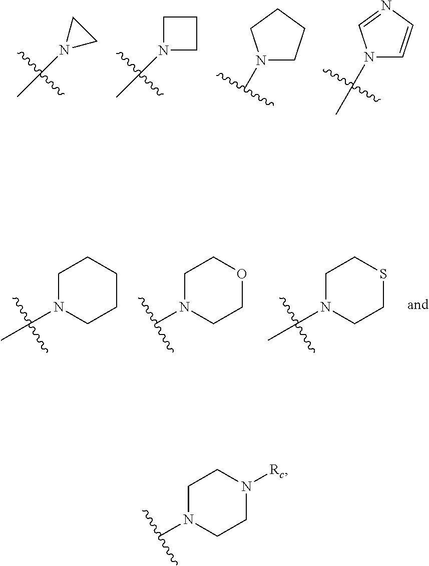
in which R₁ is H, Me, Et, n-Pr, i-Pr, n-Bu, i-Bu, t-Bu, CH₂CMe₃, Ph, or CH₂Ph.

15. The compound of claim 2, wherein R₄ is

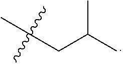 [SMILES: *CC(C)C]

16. The compound of claim 2, wherein R₄ is

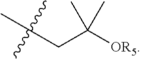

17. The compound of claim 16, wherein R₅ is H, methyl, ethyl, n-propyl, i-propyl, n-butyl, i-butyl, t-butyl, CH₂CMe₃, phenyl, CH₂-phenyl, 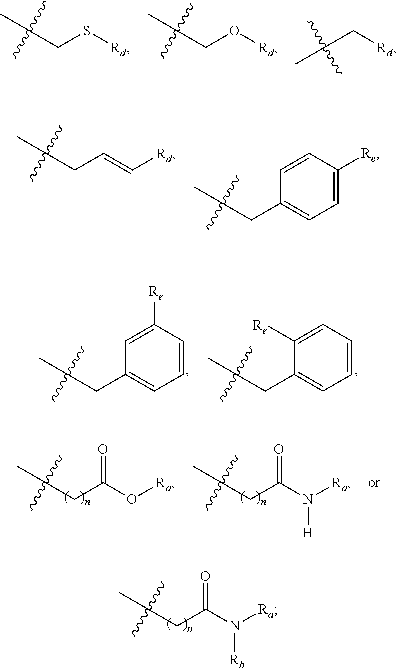
each R_{d} is independently Rₐ, ORₐ, CH₂ORₐ, 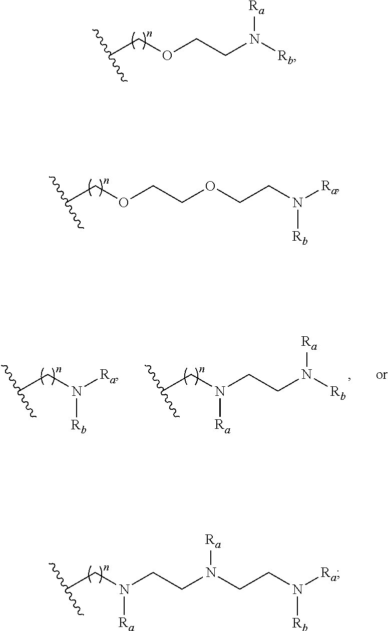
each Rₑ is independently H, Me, Et, ORₐ, CH₂ORₐ, CH₂CH₂ORₐ, 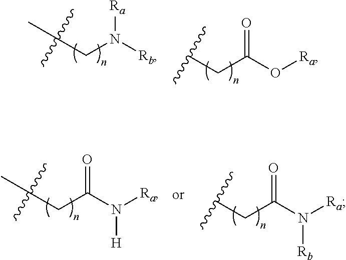
each of Rₐ and R_{b} is independently H, methyl, ethyl, n-propyl, i-propyl, n-butyl, i-butyl, t-butyl, CH₂CMe₃, phenyl, CH₂-phenyl, 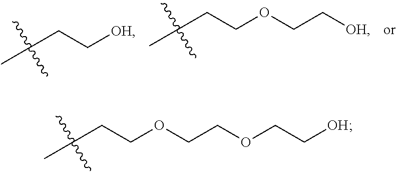
or Rₐ and R_{b}, together with the nitrogen atom to which they are attached 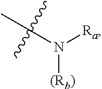
form a heterocycle selected from 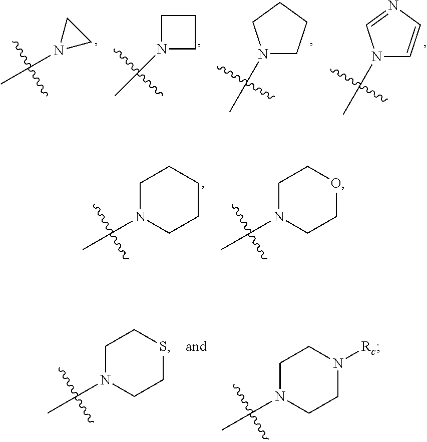
R_{c} is H, methyl, ethyl, n-propyl, i-propyl, n-butyl, i-butyl, t-butyl, CH₂CMe₃, phenyl, or CH₂-phenyl; and
each n is independently 1, 2, 3, 4, 5 or 6.

18. The compound of claim 2, wherein R₄ is

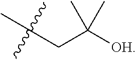 [SMILES: *CC(C)(C)O]

19. The compound of claim 2, wherein R₄ is

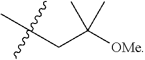 [SMILES: *CC(C)(C)OC]

20. The compound of claim 2, wherein R₄ is

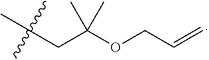 [SMILES: *CC(C)(C)OCC=C]

21. The compound of claim 1, having the following chemical structure: 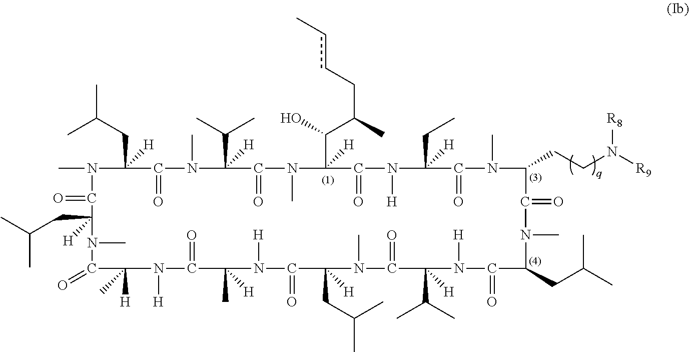
wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl, 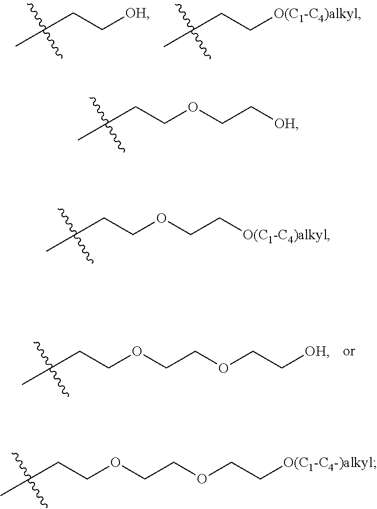
or R₈ and R₉, together with the nitrogen atom to which they are attached 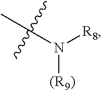
form a heterocycle selected from 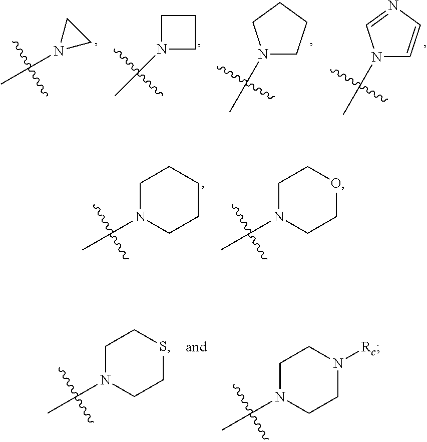
R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

22. The compound of claim 1, having the following chemical structure: 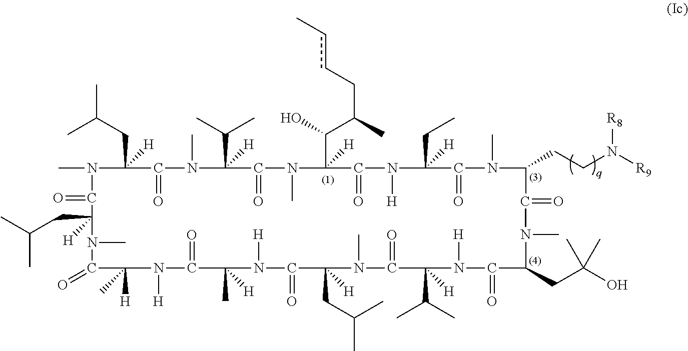
wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl, 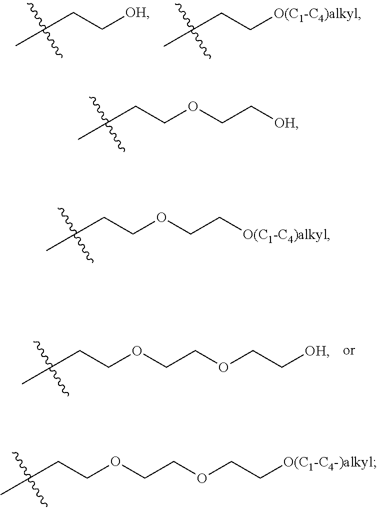
or R₈ and R₉, together with the nitrogen atom to which they are attached 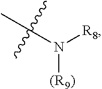
form a heterocycle selected from 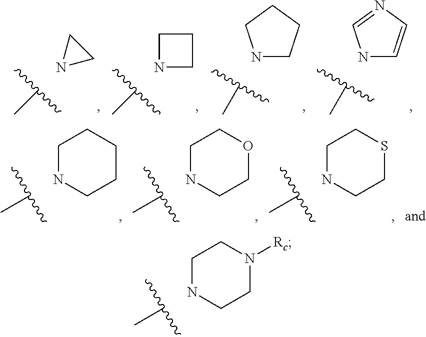
R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

23. The compound of claim 1, having the following chemical structure: 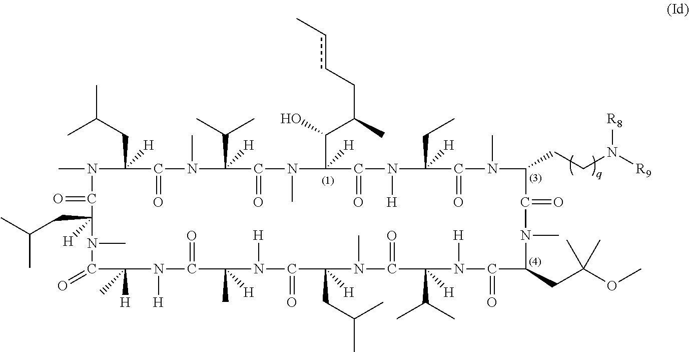
wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl, 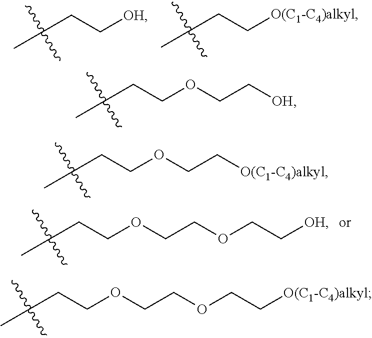
or R₈ and R₉, together with the nitrogen atom to which they are attached 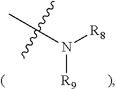
form a heterocycle selected from 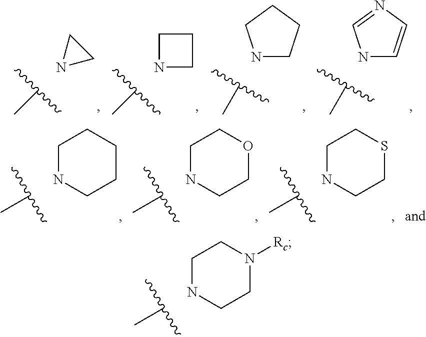
R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

24. The compound of claim 1, having the following chemical structure: 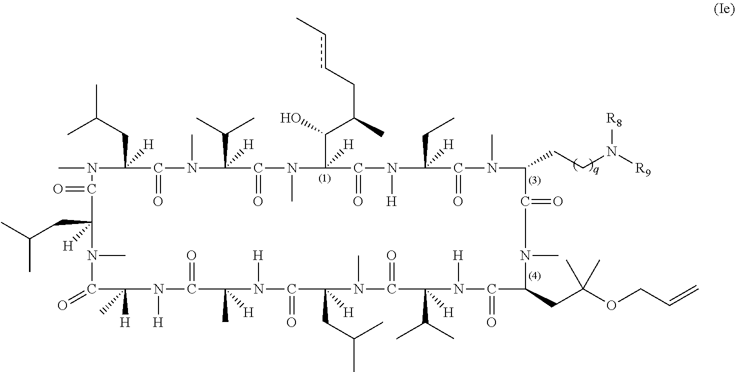
wherein ∥ represents a single bond or a double bond;
each of R₈ and R₉ is independently H, methyl, ethyl, n-propyl, i-propyl, n-butyl, i-butyl, t-butyl, CH₂CMe₃, phenyl, CH₂-phenyl, 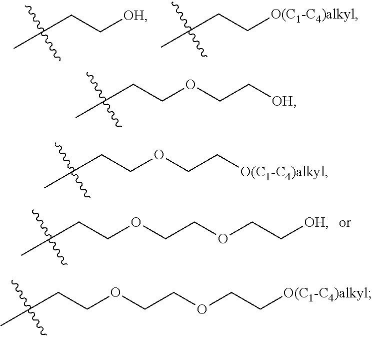
or R₈ and R₉, together with the nitrogen atom to which they are attached 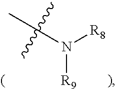
form a heterocycle selected from 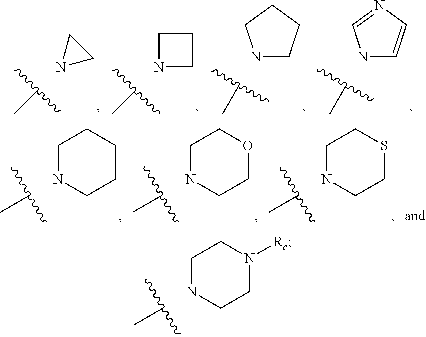
R_{c} is H, methyl, ethyl, n-propyl, i-propyl, n-butyl, i-butyl, t-butyl, CH₂CMe₃, phenyl, or CH₂-phenyl; and
q is an integer of 0, 1, 2, 3, 4, or 5.

25. A compound selected from:
[(R)-2-(N,N-Dimethylamino)ethyl-Sar]-3-cyclosporin;
[(R)-2-(N,N-Diethylamino)ethyl-Sar]-3-cyclosporin;
[(R)-2-(N-Piperidinyl)ethyl-Sar]-3-cyclosporin;
[(R)-2-(N-Thiomorpholino)ethyl-Sar]-3-cyclosporin;
[(R)-2-(N-Morpholino)ethyl-Sar]-3-cyclosporin;
[(R)-3-(N,N-Dimethylamino)propyl-Sar]-3-cyclosporin;
[(R)-3-(N,N-Diethylamino)propyl-Sar]-3-cyclosporin;
[(R)-3-(N-Piperidinyl)propyl-Sar]-3-cyclosporin;
[(R)-3-(N-Thiomorpholino)propyl-Sar]-3-cyclosporin;
[(R)-3-(N-Morpholino)propyl-Sar]-3-cyclosporin;
[(R)-4-(N,N-Dimethylamino)butyl-Sar]-3-cyclosporin;
[(R)-4-(N,N-Diethylamino)butyl-Sar]-3-cyclosporin;
[(R)-4-(N-Piperidinyl)butyl-Sar]-3-cyclosporin;
[(R)-4-(N-Thiomorpholino)butyl-Sar]-3-cyclosporin;
[(R)-4-(N-Morpholino)butyl-Sar]-3-cyclosporin;
[(R)-5-(N,N-Dimethylamino)pentyl-Sar]-3-cyclosporin;
[(R)-5-(N,N-Diethylamino)pentyl-Sar]-3-cyclosporin;
[(R)-5-(N-Piperidinyl)pentyl-Sar]-3-cyclosporin;
[(R)-5-(N-Thiomorpholino)pentyl-Sar]-3-cyclosporin;
[(R)-5-(N-Morpholino)pentyl-Sar]-3-cyclosporin;
[(R)-2-(N,N-Dimethylamino)ethyl-Sar]-3-[(γ-hydroxy)-NMeLeu]-4-cyclosporin;
[(R)-2-(N,N-Diethylamino)ethyl-Sar]-3-[(γ-hydroxy)-NMeLeu]-4-cyclosporin;
[(R)-2-(N-Piperidinyl)ethyl-Sar]-3-[(γ-hydroxy)-NMeLeu]-4-cyclosporin;
[(R)-2-(N-Thiomorpholino)ethyl-Sar]-3-[(γ-hydroxy)-NMeLeu]-4-cyclosporin;
[(R)-2-(N-Morpholino)ethyl-Sar]-3-[(γ-hydroxy)-NMeLeu]-4-cyclosporin;
[(R)-3-(N,N-Dimethylamino)propyl-Sar]-3-[(γ-hydroxy)-NMeLeu]-4-cyclosporin;
[(R)-3-(N,N-Diethylamino)propyl-Sar]-3-[(γ-hydroxy)-NMeLeu]-4-cyclosporin;
[(R)-3-(N-Piperidinyl)propyl-Sar]-3-[(γ-hydroxy)-NMeLeu]-4-cyclosporin;
[(R)-3-(N-Thiomorpholino)propyl-Sar]-3-[(γ-hydroxy)-NMeLeu]-4-cyclosporin;
[(R)-3-(N-Morpholino)propyl-Sar]-3-[(γ-hydroxy)-NMeLeu]-4-cyclosporin;
[(R)-4-(N,N-Dimethylamino)butyl-Sar]-3-[(γ-hydroxy)-NMeLeu]-4-cyclosporin;
[(R)-4-(N,N-Diethylamino)butyl-Sar]-3-[(γ-hydroxy)-NMeLeu]-4-cyclosporin;
[(R)-4-(N-Piperidinyl)butyl-Sar]-3-[(γ-hydroxy)-NMeLeu]-4-cyclosporin;
[(R)-4-(N-Thiomorpholino)butyl-Sar]-3-[(γ-hydroxy)-NMeLeu]-4-cyclosporin;
[(R)-4-(N-Morpholino)butyl-Sar]-3-[(γ-hydroxy)-NMeLeu]-4-cyclosporin;
[(R)-5-(N,N-Dimethylamino)pentyl-Sar]-3-[(γ-hydroxy)-NMeLeu]-4-cyclosporin;
[(R)-5-(N,N-Diethylamino)pentyl-Sar]-3-[(γ-hydroxy)-NMeLeu]-4-cyclosporin;
[(R)-5-(N-Piperidinyl)pentyl-Sar]-3-[(γ-hydroxy)-NMeLeu]-4-cyclosporin;
[(R)-5-(N-Thiomorpholino)pentyl-Sar]-3-[(γ-hydroxy)-NMeLeu]-4-cyclosporin;
[(R)-5-(N-Morpholino)pentyl-Sar]-3-[(γ-hydroxy)-NMeLeu]-4-cyclosporin;
[(R)-2-(N,N-Dimethylamino)ethyl-Sar]-3-[(γ-methoxy)-NMeLeu]-4-cyclosporin;
[(R)-2-(N,N-Diethylamino)ethyl-Sar]-3-[(γ-methoxy)-NMeLeu]-4-cyclosporin;
[(R)-2-(N-Piperidinyl)ethyl-Sar]-3-[(γ-methoxy)-NMeLeu]-4-cyclosporin;
[(R)-2-(N-Thiomorpholino)ethyl-Sar]-3-[(γ-methoxy)-NMeLeu]-4-cyclosporin;
[(R)-2-(N-Morpholino)ethyl-Sar]-3-[(γ-methoxy)-NMeLeu]-4-cyclosporin;
[(R)-3-(N,N-Dimethylamino)propyl-Sar]-3-[(γ-methoxy)-NMeLeu]-4-cyclosporin;
[(R)-3-(N,N-Diethylamino)propyl-Sar]-3-[(γ-methoxy)-NMeLeu]-4-cyclosporin;
[(R)-3-(N-Piperidinyl)propyl-Sar]-3-[(γ-methoxy)-NMeLeu]-4-cyclosporin;
[(R)-3-(N-Thiomorpholino)propyl-Sar]-3-[(γ-methoxy)-NMeLeu]-4-cyclosporin;
[(R)-3-(N-Morpholino)propyl-Sar]-3-[(γ-methoxy)-NMeLeu]-4-cyclosporin;
[(R)-4-(N,N-Dimethylamino)butyl-Sar]-3-[(γ-methoxy)-NMeLeu]-4-cyclosporin;
[(R)-4-(N,N-Diethylamino)butyl-Sar]-3-[(γ-methoxy)-NMeLeu]-4-cyclosporin;
[(R)-4-(N-Piperidinyl)butyl-Sar]-3-[(γ-methoxy)-NMeLeu]-4-cyclosporin;
[(R)-4-(N-Thiomorpholino)butyl-Sar]-3-[(γ-methoxy)-NMeLeu]-4-cyclosporin;
[(R)-4-(N-Morpholino)butyl-Sar]-3-[(γ-methoxy)-NMeLeu]-4-cyclosporin;
[(R)-5-(N,N-Dimethylamino)pentyl-Sar]-3-[(γ-methoxy)-NMeLeu]-4-cyclosporin;
[(R)-5-(N,N-Diethylamino)pentyl-Sar]-3-[(γ-methoxy)-NMeLeu]-4-cyclosporin;
[(R)-5-(N-Piperidinyl)pentyl-Sar]-3-[(γ-methoxy)-NMeLeu]-4-cyclosporin;
[(R)-5-(N-Thiomorpholino)pentyl-Sar]-3-[(γ-methoxy)-NMeLeu]-4-cyclosporin;
[(R)-5-(N-Morpholino)pentyl-Sar]-3-[(γ-methoxy)-NMeLeu]-4-cyclosporin;
[(R)-2-(N,N-Dimethylamino)ethyl-Sar]-3-[(γ-allyloxy)-NMeLeu]-4-cyclosporin;
[(R)-2-(N,N-Diethylamino)ethyl-Sar]-3-[(γ-allyloxy)-NMeLeu]-4-cyclosporin;
[(R)-2-(N-Piperidinyl)ethyl-Sar]-3-[(γ-allyloxy)-NMeLeu]-4-cyclosporin;
[(R)-2-(N-Thiomorpholino)ethyl-Sar]-3-[(γ-allyloxy)-NMeLeu]-4-cyclosporin;
[(R)-2-(N-Morpholino)ethyl-Sar]-3-[(γ-allyloxy)-NMeLeu]-4-cyclosporin;
[(R)-3-(N,N-Dimethylamino)propyl-Sar]-3-[(γ-allyloxy)-NMeLeu]-4-cyclosporin;
[(R)-3-(N,N-Diethylamino)propyl-Sar]-3-[(γ-allyloxy)-NMeLeu]-4-cyclosporin;
[(R)-3-(N-Piperidinyl)propyl-Sar]-3-[(γ-allyloxy)-NMeLeu]-4-cyclosporin;
[(R)-3-(N-Thiomorpholino)propyl-Sar]-3-[(γ-allyloxy)-NMeLeu]-4-cyclosporin;
[(R)-3-(N-Morpholino)propyl-Sar]-3-[(γ-allyloxy)-NMeLeu]-4-cyclosporin;
[(R)-4-(N,N-Dimethylamino)butyl-Sar]-3-[(γ-allyloxy)-NMeLeu]-4-cyclosporin;
[(R)-4-(N,N-Diethylamino)butyl-Sar]-3-[(γ-allyloxy)-NMeLeu]-4-cyclosporin;
[(R)-4-(N-Piperidinyl)butyl-Sar]-3-[(γ-allyloxy)-NMeLeu]-4-cyclosporin;
[(R)-4-(N-Thiomorpholino)butyl-Sar]-3-[(γ-allyloxy)-NMeLeu]-4-cyclosporin;
[(R)-4-(N-Morpholino)butyl-Sar]-3-[(γ-allyloxy)-NMeLeu]-4-cyclosporin;
[(R)-5-(N,N-Dimethylamino)pentyl-Sar]-3-[(γ-allyloxy)-NMeLeu]-4-cyclosporin;
[(R)-5-(N,N-Diethylamino)pentyl-Sar]-3-[(γ-allyloxy)-NMeLeu]-4-cyclosporin;
[(R)-5-(N-Piperidinyl)pentyl-Sar]-3-[(γ-allyloxy)-NMeLeu]-4-cyclosporin;
[(R)-5-(N-Thiomorpholino)pentyl-Sar]-3-[(γ-allyloxy)-NMeLeu]-4-cyclosporin;
[(R)-5-(N-Morpholino)pentyl-Sar]-3-[(γ-allyloxy)-NMeLeu]-4-cyclosporin;
[(R)-2-(N,N-Dimethylamino)ethyl-Sar]-3-[NMeVal]-4-cyclosporin;
[(R)-2-(N,N-Diethylamino)ethyl-Sar]-3-[NMeVal]-4-cyclosporin;
[(R)-2-(N-Piperidinyl)ethyl-Sar]-3-[NMeVal]-4-cyclosporin;
[(R)-2-(N-Thiomorpholino)ethyl-Sar]-3-[NMeVal]-4-cyclosporin;
[(R)-2-(N-Morpholino)ethyl-Sar]-3-[NMeVal]-4-cyclosporin;
[(R)-3-(N,N-Dimethylamino)propyl-Sar]-3-[NMeVal]-4-cyclosporin;
[(R)-3-(N,N-Diethylamino)propyl-Sar]-3-[NMeVal]-4-cyclosporin;
[(R)-3-(N-Piperidinyl)propyl-Sar]-3-[NMeVal]-4-cyclosporin;
[(R)-3-(N-Thiomorpholino)propyl-Sar]-3-[NMeVal]-4-cyclosporin;
[(R)-3-(N-Morpholino)propyl-Sar]-3-[NMeVal]-4-cyclosporin;
[(R)-4-(N,N-Dimethylamino)butyl-Sar]-3-[NMeVal]-4-cyclosporin;
[(R)-4-(N,N-Diethylamino)butyl-Sar]-3-[NMeVal]-4-cyclosporin;
[(R)-4-(N-Piperidinyl)butyl-Sar]-3-[NMeVal]-4-cyclosporin;
[(R)-4-(N-Thiomorpholino)butyl-Sar]-3-[NMeVal]-4-cyclosporin;
[(R)-4-(N-Morpholino)butyl-Sar]-3-[NMeVal]-4-cyclosporin;
[(R)-5-(N,N-Dimethylamino)pentyl-Sar]-3-[NMeVal]-4-cyclosporin;
[(R)-5-(N,N-Diethylamino)pentyl-Sar]-3-[NMeVal]-4-cyclosporin;
[(R)-5-(N-Piperidinyl)pentyl-Sar]-3-[NMeVal]-4-cyclosporin;
[(R)-5-(N-Thiomorpholino)pentyl-Sar]-3-[NMeVal]-4-cyclosporin;
[(R)-5-(N-Morpholino)pentyl-Sar]-3-[NMeVal]-4-cyclosporin;
[(R)-2-(N,N-Dimethylamino)ethyl-Sar]-3-[NMeIle]-4-cyclosporin;
[(R)-2-(N,N-Diethylamino)ethyl-Sar]-3-[NMeIle]-4-cyclosporin;
[(R)-2-(N-Piperidinyl)ethyl-Sar]-3-[NMeIle]-4-cyclosporin;
[(R)-2-(N-Thiomorpholino)ethyl-Sar]-3-[NMeIle]-4-cyclosporin;
[(R)-2-(N-Morpholino)ethyl-Sar]-3-[NMeIle]-4-cyclosporin;
[(R)-3-(N,N-Dimethylamino)propyl-Sar]-3-[NMeIle]-4-cyclosporin;
[(R)-3-(N,N-Diethylamino)propyl-Sar]-3-[NMeIle]-4-cyclosporin;
[(R)-3-(N-Piperidinyl)propyl-Sar]-3-[NMeIle]-4-cyclosporin;
[(R)-3-(N-Thiomorpholino)propyl-Sar]-3-[NMeIle]-4-cyclosporin;
[(R)-3-(N-Morpholino)propyl-Sar]-3-[NMeIle]-4-cyclosporin;
[(R)-4-(N,N-Dimethylamino)butyl-Sar]-3-[NMeIle]-4-cyclosporin;
[(R)-4-(N,N-Diethylamino)butyl-Sar]-3-[NMeIle]-4-cyclosporin;
[(R)-4-(N-Piperidinyl)butyl-Sar]-3-[NMeIle]-4-cyclosporin;
[(R)-4-(N-Thiomorpholino)butyl-Sar]-3-[NMeIle]-4-cyclosporin;
[(R)-4-(N-Morpholino)butyl-Sar]-3-[NMeIle]-4-cyclosporin;
[(R)-5-(N,N-Dimethylamino)pentyl-Sar]-3-[NMeIle]-4-cyclosporin;
[(R)-5-(N,N-Diethylamino)pentyl-Sar]-3-[NMeIle]-4-cyclosporin;
[(R)-5-(N-Piperidinyl)pentyl-Sar]-3-[NMeIle]-4-cyclosporin;
[(R)-5-(N-Thiomorpholino)pentyl-Sar]-3-[NMeIle]-4-cyclosporin; and
[(R)-5-(N-Morpholino)pentyl-Sar]-3-[NMeIle]-4-cyclosporin, or a pharmaceutically acceptable salt thereof.

26. A compound having the following structure:

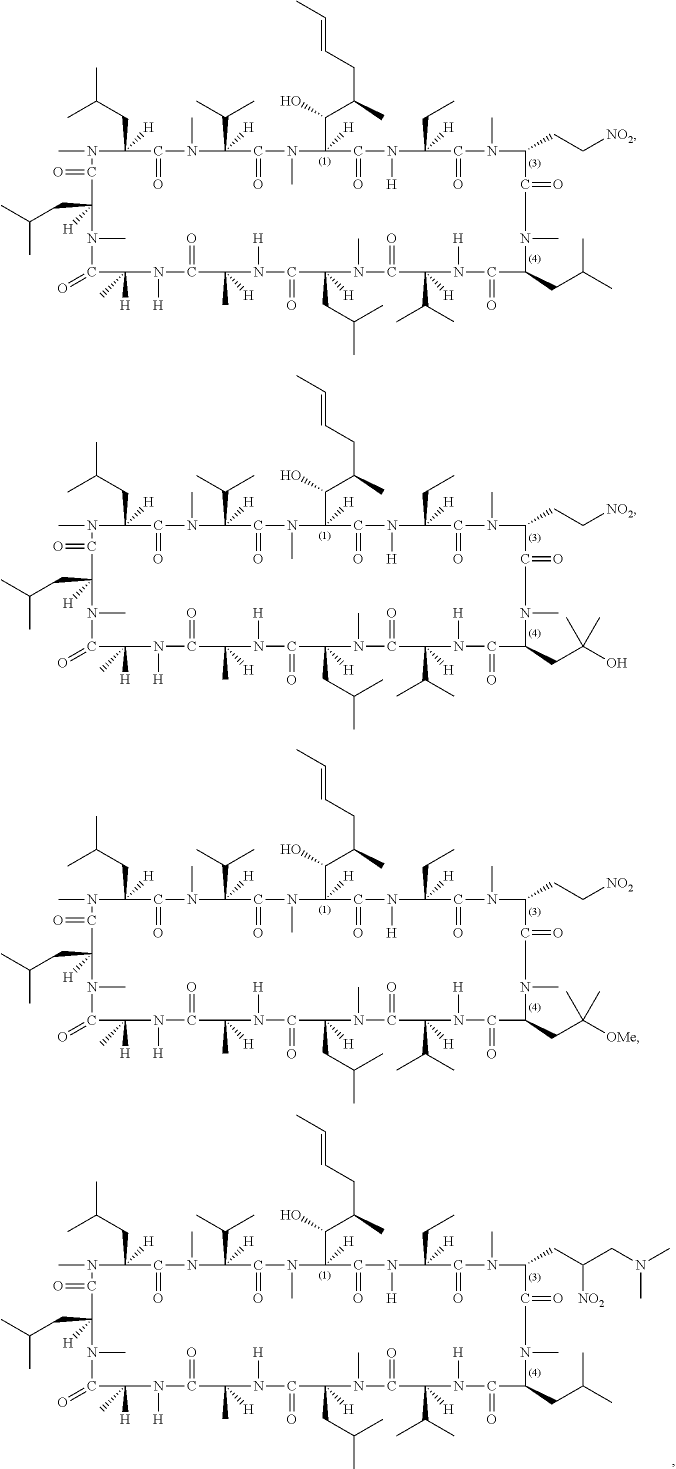

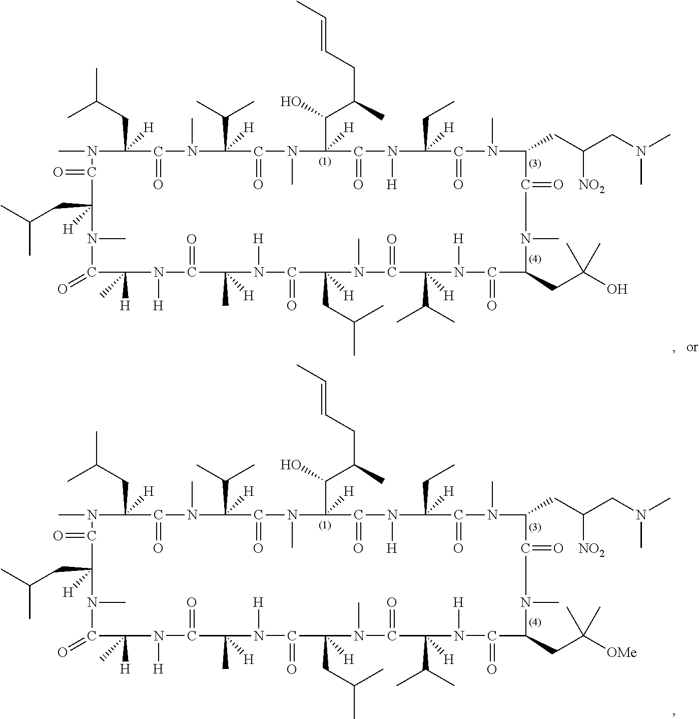

27. A pharmaceutical composition comprising at least one compound according to claim 1 or a pharmaceutically-acceptable carrier or diluent.

28. A method for treating or preventing a viral infection in a mammalian species in need thereof, the method comprising administering to the mammalian species a therapeutically effective amount of at least one compound according to claim 1.

29. A method for treating or preventing hepatitis C virus infection in a mammalian species in need thereof, the method comprising administering to the mammalian species a therapeutically effective amount of at least one compound according to claim 1.

30. A method for treating or preventing hepatitis B virus infection in a mammalian species in need thereof, the method comprising administering to the mammalian species a therapeutically effective amount of at least one compound according to claim 1.

31. A method for treating or preventing HIV virus infection in a mammalian species in need thereof, the method comprising administering to the mammalian species a therapeutically effective amount of at least one compound according to claim 1.

32. A method for preparing a compound of formula (II), 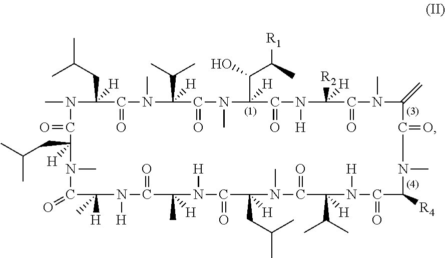
wherein
R₁ is n-butyl or (E)-but-2-enyl;
R₂ is ethyl, 1-hydroxyethyl, isopropyl or n-propyl;
R₄ is 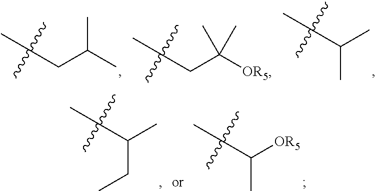
R₅ is:
H;
(C₁-C₆)alkyl, optionally substituted by one or more groups R₇ which may be the same or different;
(C₂-C₆)alkenyl, optionally substituted by one or more groups which may be the same or different selected from hydroxy, (C₁-C₆)alkyl, aryl (e.g., phenyl), (CH₂)ₚOR_{A}, O(CH₂)ₘOH, O(CH₂)ₘO(CH₂)ₘOH, O(CH₂)ₘNR_{A}R_{B}, O(CH₂)ₘO(CH₂)ₘNR_{A}R_{B}, (CH₂)ₚNR_{A}R_{B}, (CH₂)ₚNR_{c}(CH₂)ₘNR_{A}R_{B}, (CH₂)ₚNR_{C}(CH₂)ₘNR_{C}(CH₂)ₘNR_{A}R_{B}, (CH₂)ₚC(═O)NR_{A}R_{B}, (CH₂)ₚC(═O)OR_{A};
(C₂-C₆)alkynyl, optionally substituted by one or one or more groups which may be the same or different selected from halogen, hydroxy, amino, monoalkylamino and dialkylamino;
(C₃-C₇)cycloalkyl, optionally substituted by one or more groups which may be the same or different selected from halogen, hydroxy, amino, monoalkylamino and dialkylamino;
phenyl or CH₂-phenyl, optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B}, (CH₂)ₚC(═O)OR_{A};
each occurrence of R₇ is independently halogen, hydroxy, aryl (e.g., phenyl), S(C₁-C₆)alkyl, SR_{A}, OR_{A}, O(CH₂)ₘOH, O(CH₂)ₘO(CH₂)ₘOH, O(CH₂)ₘO(C₁-C₆)alkyl, O(CH₂)ₘO(CH₂)ₘO(C₁-C₆)alkyl, C(═O)OR_{A}, C(═O)NR_{A}R_{B}, NR_{A}R_{B}, O(CH₂)ₘNR_{A}R_{B}, O(CH₂)ₘO(CH₂)ₘNR_{A}R_{B}, NR_{C}(CH₂)ₘNR_{A}R_{B}, or NR_{c}(CH₂)ₘNR_{c}(CH₂)ₘNR_{A}R_{B}, wherein said aryl or phenyl is optionally substituted by one or more groups which may be the same or different selected from halogen, hydroxy, (C₁-C₆)alkyl, (CH₂)ₚOR_{A}, (CH₂)ₚNR_{A}R_{B}, (CH₂)ₚC(═O)NR_{A}R_{B} and (CH₂)ₚC(═O)OR_{A};
comprising contacting a compound of formula (III), 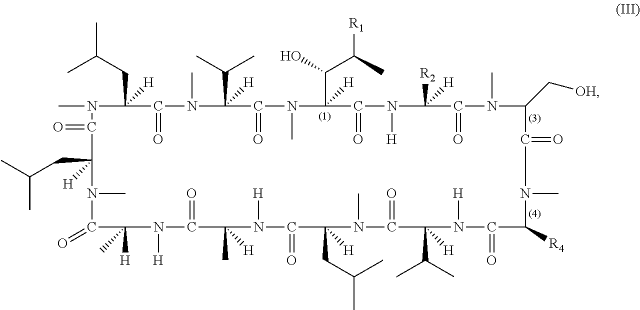
wherein R₁, R₂ and R₄ are defined as hereinabove,
with a reagent selected from: (1) MsCl or TsCl; (2) CBr₄/PPh₃; and (3) 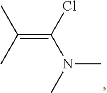
to provide the compound of formula (II).

33. The method of claim 32, wherein R₄ is

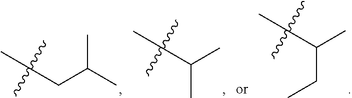

34. The method of claim 32, wherein R₄ is 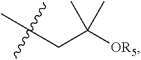
in which R₅ is H.
